(19) United States
(12) Patent Application Publication
(10) Pub. No.: US 2017/0051350 A1
(43) Pub. Date: Feb. 23, 2017

(54) METHOD AND SYSTEM TO PREDICT RESPONSE TO TREATMENTS FOR MENTAL DISORDERS

(71) Applicant: Pathway Genomics Corporation, San Diego, CA (US)

(72) Inventors: Guangdan Zhu, San Diego, CA (US); Cindy Wang, San Diego, CA (US); Tanya Moreno, San Diego, CA (US); Andrew Hellman, San Diego, CA (US); Alok Tomar, San Diego, CA (US); Svetlana Ivanova Gramatikova, San Diego, CA (US); Aditi Chawla, San Diego, CA (US); Russell Kuo-fu Chan, San Diego, CA (US); Adriana Del Tredici, San Diego, CA (US); Adrian Vilalta, San Diego, CA (US); K. David Becker, San Diego, CA (US); Michael Nova, San Diego, CA (US)

(21) Appl. No.: 15/143,263

(22) Filed: Apr. 29, 2016

Related U.S. Application Data
(63) Continuation of application No. 13/917,573, filed on Jun. 13, 2013, now abandoned.
(60) Provisional application No. 61/800,206, filed on Mar. 15, 2013.
(60) Provisional application No. 61/800,278, filed on Mar. 15, 2013.

(51) Int. Cl. C12Q 1/68; G06F 19/00

(52) U.S. Cl. CPC C12Q 1/6883; G06F 19/3431; G06F 19/704; C12Q 2600/106; C12Q 2600/156

(57) ABSTRACT

The present inventions relates to methods and assays to predict the response of an individual to a psychiatric treatment and to a method to improve medical treatment of a disorder, which is responsive to treatment with a psychiatric treatment.

### FIELD OF THE INVENTION

The invention relates to methods and assays to predict the response of an individual to a treatment for a mental disorder and to a method to improve medical treatment of a disorder, which is responsive to treatment with a psychiatric medication.

### RELATED APPLICATIONS

The present application claims priority to U.S. Provisional Patent Application Ser. No. 61/800,206, “Method And System To Predict Response To Treatments For Mental Disorders”, filed Mar. 15, 2013, the contents of which are hereby incorporated by reference in their entirety. The present application also claims priority to U.S. Provisional Patent Application Ser. No. 61/800,278, “Method And System To Predict Response To Treatments For Mental Disorders”, filed Mar. 15, 2013, the contents of which are hereby incorporated by reference in their entirety.

### BACKGROUND OF THE INVENTION

Major depressive disorder (MDD) is currently the leading cause of disability in North America as well as other countries and, according to the WHO, may become the second leading cause of disability worldwide (after heart disease) by the year 2020. Over the years, the elusive and highly variable nature of psychiatric disorders has led to drug therapy treatment that largely relies on empiricism to ascertain individual patient differences. This empirical approach has resulted in a high rate of refractory and adverse responses to drug therapies, rendering treatment of MDD one of the most significant challenges in psychiatry.

The genetic make-up of a person can contribute to the individually different responses of persons to a medicine (Roses, Nature 405:857-865, 2000). Examples of genetic factors, which determine drug tolerance, are drug allergies and severely reduced metabolism due to genetic absence of suitable enzymes. A case of a lethal lack of metabolism due to cytochrome P-450 2D6 genetic deficiency is reported by Sallee et at J Child & Adolesc. Psychopharmacol, 10: 27-34, 2000. The metabolic enzymes in the liver occur in polymorphic variants, causing some persons to metabolize certain drugs slowly and making them at risk for side effects due to excessively high plasma drug levels.

Both published literature studies and clinical experience reveal great variability in an individual's response to psychotropic drug treatment with regard to drug metabolism, side effects and efficacy.

### SUMMARY OF THE INVENTION

The present invention is related to methods and systems to the present invention for predicting an individual's likely response to a psychiatric medication comprising genotyping (including sequencing) genetic variations in an individual to determine the individual's propensity for 1) metabolizing a psychiatric medication, 2) likely response to a medication and 3) adverse reaction to a medication; and the software and algorithms to analyze the genetic information. In particular, the invention comprises analyzing a biological sample provided by an individual, typically a patient or an individual diagnosed with a particular disorder, determining the individual's likely response to a particular treatment, more specifically a psychiatric medication, and thereafter displaying, or further, recommending a plan of action or inaction. In particular, the present invention provides a grading method and system to profile an individual's response to one or more psychiatric medications. In an alternate embodiment, the present invention is directed to a method and system to recommend psychiatric medications suitable for the individual.

These methods to identify gene mutation variants are not limited by the technique that is used to identify the mutation of the gene of interest. Methods for measuring gene mutations are well known in the art and include, but are not limited to, immunological assays, nuclease protection assays, northern blots, in situ hybridization, Polymerase Chain Reaction (PCR) such as reverse transcriptase Polymerase Chain Reaction (RT-PCR) or Real-Time Polymerase Chain Reaction, expressed sequence tag (EST) sequencing, cDNA microarray hybridization or gene chip analysis, subtractive cloning, Serial Analysis of Gene Expression (SAGE), Massively Parallel Signature Sequencing (MPSS), and Sequencing-By-Synthesis (SBS).

After a patient has been identified as likely to be responsive to the therapy based on the identity of one or more of the genetic markers identified herein, the method may further comprise administering or delivering an effective amount of a treatment or an alternative treatment, to the patient, based on the outcome of the determination. Methods of administration of pharmaceuticals and biologicals are known in the art and are incorporated herein by reference.

It is conceivable that one of skill in the art will be able to analyze and identify genetic markers in situ at some point in the future. Accordingly, the inventions of this application are not to be limited to requiring isolation of the genetic material prior to analysis.

These methods also are not limited by the technique that is used to identify the polymorphism of interest. Suitable methods include but are not limited to the use of hybridization probes, antibodies, primers for PCR analysis, and gene chips, slides and software for high throughput analysis. Additional genetic markers can be assayed and used as negative controls.

This invention also provides a panel, kit, gene chip and software for patient sampling and performance of the methods of this invention. The kits contain gene chips, slides, software, probes or primers that can be used to amplify and/or for determining the molecular structure, mutations, or expression level of the genetic markers identified above. Instructions for using the materials to carry out the methods are further provided.

This invention also provides for a panel of genetic markers selected from, but not limited to the genetic polymorphisms identified herein or in combination with each other. The panel comprises probes or primers that can be used to amplify and/or for determining the molecular structure of the polymorphisms identified above. The probes or primers can be used for all RT-PCR methods as well as by a solid phase support such as, but not limited to a gene chip or microarray. The probes or primers can be detectably labeled. This aspect of the invention is a means to identify the genotype of a patient sample for the genes of interest identified above.

### BRIEF DESCRIPTION OF THE DRAWINGS

FIG. 1 displays the interaction of an individual and his caregiver in the system.

FIG. 2 describes the mechanism for providing warnings or recommendations to particular psychiatric treatments based on the efficacy of a particular treatment balanced against any potential conflicts or problems as they relate to the genotype of an individual.

FIG. 3 describes the process for a caregiver in interacting with the system.

FIG. 4 is an illustration of data stores accessed to generate a recommendation for treatments.

FIG. 5 is an illustration of a of a computer system that can perform the methods of the invention.

FIG. 6 is a diagram illustrating portals for interacting with the system for an individual (or their caregiver).

### DETAILED DESCRIPTION OF THE PREFERRED EMBODIMENTS

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Throughout this disclosure, various publications, patents and published patent specifications are referenced by an identifying citation. The disclosures of these publications, patents and published patent specifications are hereby incorporated by reference in their entirety into the present disclosure to more fully describe the state of the art to which this invention pertains.

### DEFINITIONS

The term “disease state” is used herein to mean a biological state where one or more biological processes are related to the cause or the clinical signs of the disease. For example, a disease state can be the state of a diseased cell, a diseased organ, a diseased tissue, or a diseased multi-cellular organism. Such diseases can include, for example, schizophrenia, bipolar disorder, major depression, ADHD, autism obsessive-compulsive disorder, substance abuse, Alzheimer's disease, Mild Cognitive impairment, Parkinson's disease, stroke, vascular dementia, Huntington's disease, epilepsy and Down syndrome. A diseased state could also include, for example, a diseased protein or a diseased process, such as defects in receptor signaling, neuronal firing, and cell signaling, which may occur in several different organs.

The psychiatric disease or disorder according to the present invention may be any psychiatric or neuropsychiatric disease or disorder which includes disturbances in motivational, emotional or cognitive function, such as schizophrenia, obsessive-compulsive disorder (OCD), major depression, bipolar disorder or dementia accompanied, i.e., complicated, by aggression or affective disorder, i.e., mental disorder characterized by dramatic changes or extremes of mood, such as manic (elevated, expansive or irritable mood with hyperactivity, pressured speech and inflated self-esteem), depressive (dejected mood with disinterest in life, apathy, sleep disturbance, agitation and feelings of worthlessness or guilt) episodes, or combinations thereof. In a preferred embodiment, the psychiatric disease or disorder is schizophrenia.

A “mental disorder” or “mental illness” or “mental disease” or “psychiatric or neuropsychiatric disease or illness or disorder” refers to mood disorders (e.g., major depression, mania, and bipolar disorders), psychotic disorders (e.g., schizophrenia, schizoaffective disorder, schizophreniform disorder, delusional disorder, brief psychotic disorder, and shared psychotic disorder), personality disorders, anxiety disorders (e.g., obsessive-compulsive disorder) as well as other mental disorders such as substance-related disorders, childhood disorders, dementia, autistic disorder, adjustment disorder, delirium, multi-infarct dementia, and Tourette's disorder as described in Diagnostic and Statistical Manual of Mental Disorders, Fourth Edition, (DSM IV). Typically, such disorders have a genetic and/or a biochemical component as well.

A “mood disorder” refers to disruption of feeling tone or emotional state experienced by an individual for an extensive period of time. Mood disorders include major depression disorder (i.e., unipolar disorder), mania, dysphoria, bipolar disorder, dysthymia, cyclothymia and many others. See, e.g., Diagnostic and Statistical Manual of Mental Disorders, Fourth Edition, (DSM IV).

“Major depression disorder,” “major depressive disorder,” or “unipolar disorder” refers to a mood disorder involving any of the following symptoms: persistent sad, anxious, or “empty” mood; feelings of hopelessness or pessimism; feelings of guilt, worthlessness, or helplessness; loss of interest or pleasure in hobbies and activities that were once enjoyed, including sex; decreased energy, fatigue, being “slowed down”; difficulty concentrating, remembering, or making decisions; insomnia, early-morning awakening, or oversleeping; appetite and/or weight loss or overeating and weight gain; thoughts of death or suicide or suicide attempts; restlessness or irritability; or persistent physical symptoms that do not respond to treatment, such as headaches, digestive disorders, and chronic pain. Various subtypes of depression are described in, e.g., DSM IV.

“Bipolar disorder” is a mood disorder characterized by alternating periods of extreme moods. A person with bipolar disorder experiences cycling of moods that usually swing from being overly elated or irritable (mania) to sad and hopeless (depression) and then back again, with periods of normal mood in between. Diagnosis of bipolar disorder is described in, e.g., DSM IV. Bipolar disorders include bipolar disorder I (mania with or without major depression) and bipolar disorder II (hypomania with major depression), see, e.g., DSM IV.

“A psychotic disorder” refers to a condition that affects the mind, resulting in at least some loss of contact with reality. Symptoms of a psychotic disorder include, e.g., hallucinations, changed behavior that is not based on reality, delusions and the like. See, e.g., DSM IV. Schizophrenia, schizoaffective disorder, schizophreniform disorder, delusional disorder, brief psychotic disorder, substance-induced psychotic disorder, and shared psychotic disorder are examples of psychotic disorders.

“Schizophrenia” refers to a psychotic disorder involving a withdrawal from reality by an individual. Symptoms comprise for at least a part of a month two or more of the following symptoms: delusions (only one symptom is required if a delusion is bizarre, such as being abducted in a space ship from the sun); hallucinations (only one symptom is required if hallucinations are of at least two voices talking to one another or of a voice that keeps up a running commentary on the patient's thoughts or actions); disorganized speech (e.g., frequent derailment or incoherence); grossly disorganized or catatonic behavior; or negative symptoms, i.e., affective flattening, alogia, or avolition. Schizophrenia encompasses disorders such as, e.g., schizoaffective disorders. Diagnosis of schizophrenia is described in, e.g., DSM IV. Types of schizophrenia include, e.g., paranoid, disorganized, catatonic, undifferentiated, and residual.

An “agonist” refers to an agent that binds to a polypeptide or polynucleotide of the invention, stimulates, increases, activates, facilitates, enhances activation, sensitizes or up regulates the activity or expression of a polypeptide or polynucleotide of the invention.

An “antagonist” refers to an agent that inhibits expression of a polypeptide or polynucleotide of the invention or binds to, partially or totally blocks stimulation, decreases, prevents, delays activation, inactivates, desensitizes, or down regulates the activity of a polypeptide or polynucleotide of the invention.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators. Inhibitors are agents that, e.g., inhibit expression of a polypeptide or polynucleotide of the invention or bind to, partially or totally block stimulation or enzymatic activity, decrease, prevent, delay activation, inactivate, desensitize, or down regulate the activity of a polypeptide or polynucleotide of the invention, e.g., antagonists. Activators are agents that, e.g., induce or activate the expression of a polypeptide or polynucleotide of the invention or bind to, stimulate, increase, open, activate, facilitate, enhance activation or enzymatic activity, sensitize or up regulate the activity of a polypeptide or polynucleotide of the invention, e.g., agonists. Modulators include naturally occurring and synthetic ligands, antagonists, agonists, small chemical molecules and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide of the invention that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of a polypeptide or polynucleotide of the invention relative to the control is about 80%, optionally 50% or 25-1%. Activation is achieved when the activity value of a polypeptide or polynucleotide of the invention relative to the control is 110%, optionally 150%, optionally 200-500%, or 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

There are six main groups of psychiatric medications.
Antidepressants, which treat disparate disorders such as clinical depression, dysthymia, anxiety, eating disorders and borderline personality disorder.
Antipsychotics, which treat psychoses such as schizophrenia and mania.
Stimulants, which treat disorders such as attention deficit hyperactivity disorder and narcolepsy, and to suppress the appetite.
Anxiolytics, which treat anxiety disorders.
Mood stabilizers, which treat bipolar disorder and schizoaffective disorder.
Depressants, which are used as hypnotics, sedatives, and anesthetics.

### Antidepressants

An “antidepressant” refers to an agents typically used to treat clinical depression. Antidepressants includes compounds of different classes including, for example, selective serotonin reuptake inhibitors (e.g., Femoxetine, Citalopram (Celexa), escitalopram (Lexapro, Cipralex), paroxetine (Paxil, Seroxat), fluoxetine (Prozac), fluvoxamine (Luvox), sertraline (Zoloft, Lustral)), norepinephrine reuptake inhibitors (e.g., atomoxetine (Strattera), nisoxetine, maprotiline, reboxetine (Edronax), viloxazine (Vivalan)), Noradrenergic and specific serotonergic antidepressants (NaSSA) (e.g., mianserin (Tolvon), mirtazapine (Remeron, Avanza, Zispin)), Serotonin-norepinephrine reuptake inhibitors (e.g., Desvenlafaxine (Pristiq), duloxetine (Cymbalta), milnacipran (Ixel, Savella), venlafaxine (Effexor)), Serotonin antagonist and reuptake inhibitors (e.g., etoperidone (Axiomin, Etonin), nefazodone (Serzone, Nefadar), trazodone (Desyrel)), norepinephrine-dopamine reuptake inhibitors (e.g., Nomifensine, Bupropion (Wellbutrin, Zyban)), selective serotonin reuptake enhancers (e.g., Tianeptine (Stablon, Coaxil, Tatinol), amineptine), norepinephrine-dopamine disinhibitors (e.g., Agomelatine (Valdoxan, Melitor, Thymanax)), tricyclic antidepressants (e.g., Mazindol, Oxaprotiline, Tertiary amine tricyclic antidepressants such as Amitriptyline (Elavil, Endep), Clomipramine (Anafranil), Doxepin (Adapin, Sinequan), Imipramine (Tofranil), Lofepramine (Lomont, Gamanil), or Trimipramine (Surmontil), Secondary amine tricyclic antidepressants such as Butriptyline (Evadyne), Amoxapine, Desipramine (Norpramin), Dosulepin/Dothiepin (Prothiaden), Nortriptyline (Pamelor, Aventyl, Noritren), Protriptyline (Vivactil)), monoamine oxidase inhibitor (e.g., Isocarboxazid (Marplan), Moclobemide (Aurorix, Manerix), Phenelzine (Nardil), Pirlindole (Pirazidol), Selegiline (Eldepryl, Emsam), Tranylcypromine (Parnate)), nicotine, caffeine, cannabinoids, tricyclic antidepressants (e.g., desipramine), and dopamine reuptake inhibitors (e.g, bupropion). Typically, antidepressants of different classes exert their therapeutic effects via different biochemical pathways. Often these biochemical pathways overlap or intersect. Additional diseases or disorders often treated with antidepressants include, chronic pain, anxiety disorders, and hot flashes. Examples of antidepressant agents, without limitation, include, mirtazapine, duloxetine, venlafaxine, buspirone, bupropion, trazodone. Tricyclic antidepressants protriptyline, amitriptyline, nortriptyline, amitriptylinoxide, imipramine, clomipramine, desipramine, doxepin, trimipramine. Known drugs specifically named as SSRI are fluoxetine, fluvoxamine, citalopram, cericlamine, dapoxetine, escitalopram, femoxetine, indalpine, paroxetine, sertraline, paroxetine, ifoxetine, cyanodothiepin, zimelidine, and litoxetine.

SSRI side effects include but are not limited to: Serotonin syndrome, nausea, diarrhea, increased blood pressure, agitation, headaches anxiety, nervousness, emotional lability, increased suicidal ideation, suicide attempts, insomnia, drug interactions, neonate adverse reactions, anorexia, dry mouth, somnolence, tremors, sexual dysfunction decreased libido, asthenia, dyspepsia, dizziness, sweating, personality disorder, epistaxis, urinary frequency, menorrhagia, mania/hypomania, chills, palpitations, taste perversion, and micturition disorder drowsiness, GI irregularities, muscle weakness, long term weight gain

Tricyclic antidepressants common side effects include: dry mouth, blurred vision, drowsiness, dizziness, tremors, sexual problems, skin rash, and weight gain or loss.

MAOIs (monoamine oxidase inhibitors) side effects include: MAOI can produce a potentially lethal hypertensive reaction if taken with foods that contain excessively high levels of tyramine, such as mature cheese, cured meats or yeast extracts. Likewise, lethal reactions to both prescription and over the counter medications have occurred. Patients undergoing therapy with MAO inhibiting medications are monitored closely by their prescribing physicians, who are consulted before taking an over the counter or prescribed medication. Such patients must also inform emergency room personnel and keep information with their identification indicating that they are on MAOI. Some doctors suggest the use of medical identification tags. Although these reactions may be lethal, the total number of deaths due to interactions and dietary concerns is comparable to over-the-counter medications.

Other side effects of MAOI include: hepatitis, heart attack, stroke, and seizures. Serotonin syndrome is a side-effect of MAOIs when combined with certain medications. Moclobemide may be preferred in the elderly as its pharmacokinetics are not affected by age, is well tolerated by the elderly as well as younger adults, has few serious adverse events, and, in addition, it is as effective as other antidepressants that have more side-effects; moclobemide also has beneficial effects on cognition. A new generation of MAOIs has been introduced; moclobemide (Manerix), known as a reversible inhibitor of monoamine oxidase A (RIMA), which is as effective as SSRIs and tricyclic antidepressants, in depressive disorders, acts in a more short-lived and selective manner and does not require a special diet.

Side-effects of NaSSI may include drowsiness, increased appetite, and weight gain.

Side effects of tricyclics include increased heart rate, drowsiness, dry mouth, constipation, urinary retention, blurred vision, dizziness, confusion, and sexual dysfunction. Toxicity occurs at about ten times normal dosages; these drugs are often lethal in overdoses, as they may cause a fatal arrhythmia. However, tricyclic antidepressants are still used because of their effectiveness, especially in severe cases of major depression, their favourable price, and off label uses.

Breast cancer survivors risk having their disease come back if they use certain antidepressants while also taking the cancer prevention drug tamoxifen, according to research released in May 2009.

For bipolar depression, anti-depressant, most frequently SSRIs, can exacerbate or trigger symptoms of hypomania and mania.

The use of antidepressants during pregnancy is associated with an increased risk of spontaneous abortion.

### Antipsychotics/Neuroleptics

The terms antipsychotics/neuroleptics are used herein to mean drugs used for the treatment of psychosis, such as schizophrenia and bipolar disorder. These drugs include but are not limited to butyrophenones (e.g., Haloperidol (Haldol, Serenace), Droperidol (Droleptan, Inapsine)); phenothiazines (e.g., Chlorpromazine (Thorazine, Largactil), Fluphenazine (Prolixin), Perphenazine (Trilafon), Prochlorperazine (Compazine), Thioridazine (Mellaril), Trifluoperazine (Stelazine), Mesoridazine (Serentil), Periciazine, Promazine, Triflupromazine (Vesprin), Levomepromazine (Nozinan), Promethazine (Phenergan), Pimozide (Orap), Cyamemazine (Tercian)); thioxanthenes (e.g., Chlorprothixene (Cloxan, Taractan, Truxal), Clopenthixol (Sordinol), Flupenthixol (Depixol, Fluanxol), Thiothixene (Navane), Zuclopenthixol (Cisordinol, Clopixol, Acuphase)) atypical antipsychotic drugs risperidone (Risperdal®), olanzapine (Zyprexa®), ziprasidone (Geodone®) quetiapine, aripiprazole, iloperidone, asenapine, lurasidone, paliperidone, iloperidone, zotepine, sertindole, lorasidone, and clozapine (clozaril); the typical antipsychotic drugs haloperidol, zuclopenthixol, chlorpromazine, fluphenazine, perphenazine loxapine thiothixene and trifluperazine (Eskazinyl®); the antipsychotic drug amisulpride (Solian®); and a thioxanthene derivative such as the typical antipsychotic drugs chlorprothixene and thiothixene (Navane®), and the typical antipsychotic neuroleptic drugs flupentixol (Depixol® or Fluanxol®) and zuclopenthixol (Cisordinol®, Clopixol® or Acuphase®), available as zuclopenthixol decanoate, zuclopenthixol acetate and zuclopenthixol dihydrochloride. Other compounds include partial agonists of dopamine receptors, cannabidiols, tetrabenazine, metabotropic glutamate receptor 2 agonists, and glycine transporter 1 antagonists.

A number of harmful and undesired (adverse) effects for antipsychotics have been observed, including lowered life expectancy, extrapyramidal effects on motor control—including akathisia (an inability to sit still), trembling, and muscle weakness, weight gain, decrease in brain volume, enlarged breasts (gynecomastia) in men and milk discharge in men and women (galactorrhea due to hyperprolactinaemia), lowered white blood cell count (agranulocytosis), involuntary repetitive body movements (tardive dyskinesia), diabetes, and sexual dysfunction.

### Psychostimulants

Stimulants (also referred to as psychostimulants) are psychoactive drugs which induce temporary improvements in either mental or physical function or both. Examples of psycho stimulants to “augment” the include amphetamine (Adderall), dextroamphetamine, levoamphetamine, methamphetamine (desoxyn), methylphenidate (Ritalin), and modafinil (Provigil, Alertec). Stimulants can be addictive, and patients with a history of drug abuse are typically monitored closely or even barred from use and given an alternative.

### Anxiolytic/Anti-Anxiety Drugs

An anxiolytic (also antipanic or antianxiety agent) is a drug that inhibits anxiety, which include Benzodiazepines (e.g., Alprazolam (Xanax), Chlordiazepoxide (Librium), Clonazepam (Klonopin, Rivotril), Diazepam (Valium), Etizolam (Etilaam), Lorazepam (Ativan), Nitrazepam (Mogadon), Oxazepam (Serax), Temazepam (Restoril), Tofisopam (Emandaxin and Grandaxin)), Serotonergic antidepressants (see, e.g., SSRI's above), Afobazole, Selank, Bromantane, Azapirones (e.g., buspirone (Buspar) and tandospirone (Sediel), Gepirone (Ariza, Variza)), Zaleplon (Sonata), Barbiturates, Hydroxyzine, Pregabalin, Picamilon, Chlorpheniramine, Melatonin, BNC210 (Ironwood Pharmaceuticals), CL-218,872, L-838,417 (Merck, Sharp & Dohme), SL-651,498.

### Mood Stabilizers/Anticonvulsants

Examples of mood stabilizers include valproic acid, lithium, riluzole (rilutek), gabapentin, topiramate, valproic acid, gabapentin, lamotrigine, oxcarbazepine, carbamazepine and topiramate, as well as several Some atypical antipsychotics (risperidone, olanzapine, quetiapine, paliperidone, and ziprasidone) also have mood stabilizing effects[11] and are thus commonly prescribed even when psychotic symptoms are absent.

An antidepressant is often prescribed in addition to the mood stabilizer during depressive phases. This brings some risks, however, as antidepressants can induce mania, psychosis, and other disturbing problems in people with bipolar disorder—in particular, when taken alone, but sometimes even when used with a mood stabilizer. Antidepressants' utility in treating depression-phase bipolar disorder is unclear.

Antidepressants cause several risks when given to bipolar patients. They are ineffective in treating acute bipolar depression, preventing relapse, and can cause rapid cycling. Studies have been shown that antidepressants have no benefit versus a placebo or other treatment. Antidepressants can also lead to a higher rate of non-lethal suicidal behavior. Relapse can also be related to treatment with antidepressants. This is less likely to occur if a mood stabilizer is combined with an antidepressant, rather than an antidepressant being used alone. Evidence from previous studies shows that rapid cycling is linked to use of antidepressants. Rapid cycling is when a person with bipolar disorder experiences four or more mood episodes, such as mania or depression, within a year. These issues have become more prevalent since antidepressant medication has come into widespread use. There is a need for caution when treating bipolar patients with antidepressant medication due to the risks that they pose.

Use of mood stabilizers and anticonvulsants such as lamotrigine, carbamazapine, valproate and others may lead to chronic folate deficiency, potentiating depression. Also, “Folate deficiency may increase the risk of depression and reduce the action of antidepressants.” L-methylfolate (also formally known as 5-MTHF or Levofolinic acid), a centrally acting trimonoamine modulator, boosts the synthesis of three CNS neurotransmitters: dopamine, norepinephrine and serotonin. Mood stabilizers and anticonvulsants may interfere with folic acid absorption and L-methylfolate formation. Augmentation with the medical food L-methylfolate may improve antidepressant effects of these medicines, including lithium and antidepressants themselves, by boosting the synthesis of antidepressant neurotransmitters.

### Depressant

A depressant, or central depressant, is a drug or endogenous compound that lowers or depresses arousal levels and reduces excitability. Examples of depressants prescribed by health care providers include barbiturates, benzodiazepines, cannabis, opioids, alpha and beta blockers (Carvedilol, Propanolol, atenolol, etc.), anticholinergics (Atropine, hyoscyamine, scopolamine, etc.), anticonvulsants (Valproic acid, carbamazepine, lamotrigine, etc.), antihistamines (Diphenhydramine, doxylamine, promethazine, etc.), antipsychotics (Haloperidol, chlorpromazine, clozapine, etc.), dissociatives (Dextromethorphan, ketamine, phencyclidine, nitrous oxide, etc.), hypnotics (Zolpidem, zopiclone, chloral hydrate, chloroform, etc.), muscle relaxants (Baclofen, carisoprodol, cyclobenzaprine, etc.), and sedatives (Gamma-hydroxybutyrate, etc.).

The terms “genetic variation” or “genetic variant”, as they are used in the present description include mutations, polymorphisms and allelic variants. A variation or genetic variant is found amongst individuals within the population and amongst populations within the species.

The term “polymorphism” refers to a variation in the sequence of nucleotides of nucleic acid where every possible sequence is present in a proportion of equal to or greater than 1% of a population. A portion of a gene of which there are at least two different forms, i.e., two different nucleotide sequences, is referred to as a “polymorphic region of a gene”. A polymorphic region can be a single nucleotide, the identity of which differs in different alleles; in a particular case, when the said variation occurs in just one nucleotide (A, C, T or G) it is called a single nucleotide polymorphism (SNP).

A “polymorphic gene” refers to a gene having at least one polymorphic region.

The term “genetic mutation” refers to a variation in the sequence of nucleotides in a nucleic acid where every possible sequence is present in less than 1% of a population.

The terms “allelic variant” or “allele” are used without distinction in the present description and refer to a polymorphism that appears in the same locus in the same population.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “genotype” refers to the specific allelic composition of an entire cell or a certain gene, whereas the term “phenotype’ refers to the detectable outward manifestations of a specific genotype.

As used herein, “genotyping” a subject (or DNA sample) for a polymorphic allele of a gene (s) refers to detecting which allelic or polymorphic form (s) of the gene (s) are present in a subject (or a sample). As is well known in the art, an individual may be heterozygous or homozygous for a particular allele. More than two allelic forms may exist, thus there may be more than three possible genotypes.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

As used herein, the term “haplotype” refers to a group of closely linked alleles that are inherited together.

The expression “amplification” or “amplify” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

“Biological sample” or “sample” refers to the biological sample that contains nucleic acid taken from a fluid or tissue, secretion, cell or cell line derived from the human body. For example, samples may be taken from blood, including serum, lymphocytes, lymphoblastoid cells, fibroblasts, platelets, mononuclear cells or other blood cells, from saliva, liver, kidney, pancreas or heart, urine or from any other tissue, fluid, cell or cell line derived from the human body. For example, a suitable sample may be a sample of cells from the buccal cavity.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence that has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The term “isolated” as used herein with respect to nucleic acids, such as DNA or RNA, refers to molecules separated from other DNAs or RNAs, respectively, which are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state. The term “isolated” is also used herein to refer to polypeptides that are isolated from other cellular proteins and is meant to encompass both purified and recombinant polypeptides.

The term “mismatches” refers to hybridized nucleic acid duplexes that are not 100% homologous. The lack of total homology may be due to deletions, insertions, inversions, substitutions or frameshift mutations.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine”, “cytidine”, “guanosine”, and “thymidine” are used. It is understood that if the nucleic acid is RNA, a nucleotide having a uracil base is uridine.

The terms “oligonucleotide” or “polynucleotide”, or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

As used herein, the term “label” intends a directly or indirectly detectable compound or composition that is conjugated directly or indirectly to the composition to be detected, e.g., polynucleotide so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6 ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Iain Johnson and Michelle T. Z. Spence. (1

Molecular Probes Handbook, A Guide to Fluorescent Probes and Labeling Technologies (Invitrogen Corp; 11th ed.). (2010).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

When a genetic marker or polymorphism “is used as a basis” for selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease.

A “response” implies any kind of improvement or positive response either clinical or non-clinical such as, but not limited to, measurable evidence of diminishing disease or disease progression, complete response, partial response, stable disease, increase or elongation of progression free survival, increase or elongation of overall survival, or reduction in toxicity or side effect vulnerability.

The term “likely to respond” shall mean that the patient is more likely than not to exhibit at least one of the described treatment parameters, identified above, as compared to similarly situated patients.

As used herein, the terms “increased”, “higher”, “greater”, “faster” or similar terms in association with the ability of an individual with a certain genotype to respond to a treatment shall refer to or mean having average or above average activity (the activity associated with such terms, not meant to be positive or negative) to such treatments, (e.g., faster metabolism, increased efficacy or apposingly, increased vulnerability to side effects, or increased tolerance to treatments) in comparison to similarly situated individuals with genotype(s). Alternatively, the terms “decreased”, “lower”, “reduced” or similar terms in association with the ability of individuals with a certain genotype to respond to a treatment shall mean having less or reduced response to such treatments, increased vulnerability to side effects, or reduced tolerance to treatment in comparison to similarly situated individuals with different genotype(s).

### General Embodiments of the Invention

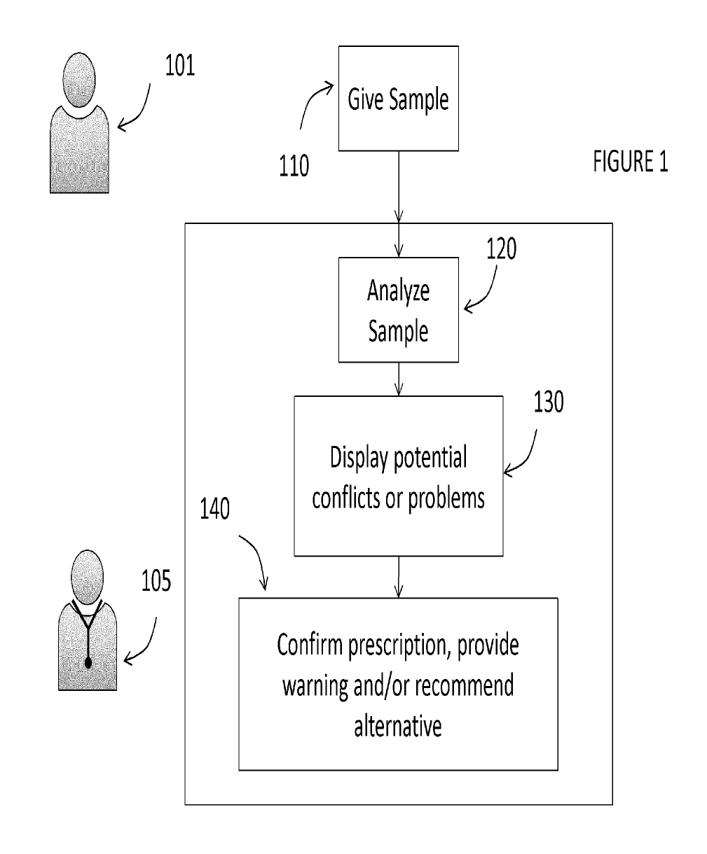

In one embodiment, as illustrated in FIG. 1, the present invention relates to systems and methods for predicting an individual's likely response to a psychiatric medication comprising genotyping genetic variations in an individual to determine the individual's propensity for 1) metabolizing a psychiatric medication, 2) likely response to a medication and 3) adverse reaction to a medication. In particular, the invention comprises analyzing a biological sample provided by an individual, typically a patient or an individual diagnosed with a particular disorder, determining the individual's likely response to a particular treatment, more specifically a psychiatric medication, and thereafter displaying, or further, recommending a plan of action or inaction. In particular, the present invention provides a grading method and system to profile an individual's response to one or more psychiatric medication. In an alternate embodiment, the present invention is directed to a method and system to recommend psychiatric medications suitable for the individual.

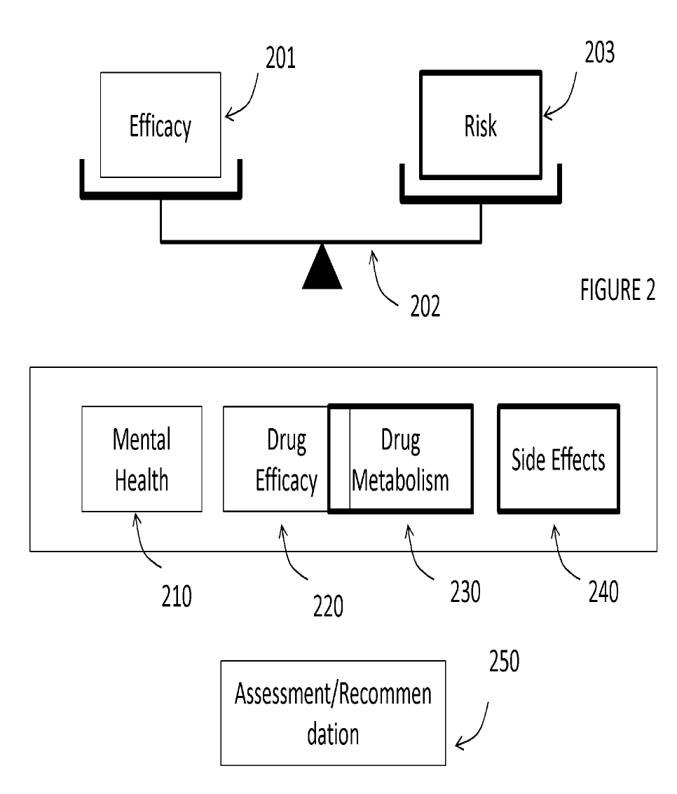

In a more preferred embodiment, as shown in FIG. 2, the present invention is directed to a method and system for analyzing an array of genetic variations related to medication or drug metabolism, drug efficacy and side effects. In a preferred method, the present invention comprises genotyping genetic variations in an individual to determine:
1) a categorical grade to the individual's likely ability to metabolize a particular psychiatric medication, a categorical grade for a psychiatric medication's potential efficacy with respect to the individual, and a categorical grade to the propensity for the individual to have a negative adverse reaction to the particular psychiatric medication,
2) aggregating the categorical grades, and thereafter identifying the least positive grade as the recommendation for the individual.
   Preferably, the individual is genotyped against a panel of at least one gene that affects the rate of drug metabolism, a panel of genes that affect a psychiatric medication's potential efficacy with respect to the individual, and a panel of genes that affect the propensity for the individual to have a negative adverse reaction to the particular psychiatric medication.

As defined herein, the term “least positive” refers to the most precautionary category or measure or assessment that can be attributed to an individual based on their potential response to psychiatric medications. For example, the assessment for an individual with respect to their response to a particular drug may be positive or normal with respect to all aspects except, for example, a potential negative adverse reaction. The potential negative reaction would be the least positive or most precautionary assessment, and would be the recommendation to the patient, e.g., the patient may be at risk for potential negative adverse reactions.

FIG. 2 can be identified as a method and system for genetically evaluating the efficacy 201 of a particular treatment for a mental disorder for an individual balanced 202 against any risks 203 associated with the use of such treatment. Once a particular disorder is identified, and preferably confirmed 210, the efficacy of the drug 220 with respect to the particular individual and the disorder, is balanced against the pharmacokinetics of the medication or drug 230 and further weighted by any potential side effects 240 that the individual or the drugs may be prone to. The disorder can be assessed by genotyping the individual to determine if they are prone to such disorder or by traditional means of diagnosing such disorders. In many cases, the pharmacokinetics of the drug will affect the efficacy of the drug, e.g., tolerance or metabolism of the drug will affect the disorder and the individual, and also the side effects or any adverse effects that may arise due to the drug lingering or affecting non-desired pathways. A recommendation or assessment 250 is made based on the weighting of these factors.

In a more preferred embodiment, the present invention comprises an algorithm or system, wherein a drug is assigned to categories such as one of the four categories below:

### 1. Use as Directed

### 2. Preferential Use

### 3. May Have Limitations

### 4. May Cause Serious Adverse Events

For example, in one embodiment, each drug is assigned to the default category, “Use as Directed”, unless it is reassigned to another category based on genetic test result(s). In case the drug can be reassigned to multiple categories because of results from multiple genetic tests, the category that invokes most precautionary measures (e.g., least positive) will apply to the drug. For instance, a drug will be assigned to the “May Cause Serious Adverse Events” category for a patient when the patient is positive for both 1) a genotype that is associated with increased response to the drug, suggesting the “Preferential Use” category, and 2) another genotype that is associated with increased risk of serious adverse events, suggesting the “May Cause Serious Adverse Events” category.

The Input of the algorithm consists of the genotyping results of the patient.

The output of the algorithm consists of the recommendation categories for all tested drugs and a text for each drug that is not assigned to the “Use as Directed” category. The text includes detailed reasons for the category assignment and, when appropriate, clinical recommendations.

The algorithm consists of:
A library of candidate recommendation category assignments for all drug-genotype combinations,
A library of texts for all drug-genotype combinations,
Rules for determining the final drug recommendation categories,
Rules for selecting texts for display in the test report, and
Rules for assessing the impact of incomplete test results.

In one embodiment, the present invention relates to a method of genotyping genetic variations in an individual, which is sufficiently sensitive, specific and reproducible as to allow its use in a clinical setting. The inventors have developed unique methodology with specifically designed primers and probes for use in the method.

Thus in one aspect, the invention comprises an in vitro method for genotyping genetic variations in an individual. The in vitro, extracorporeal method is for simultaneous sensitive, specific and reproducible genotyping of multiple human genetic variations present in one or more genes of a subject. The method of the invention allows identification of nucleotide changes, such as, insertions, duplications and deletions and the determination of the genotype of a subject for a given genetic variation.

A given gene may comprise one or more genetic variations. Thus the present methods may be used for genotyping of one or more genetic variations in one or more genes.

Thus a genetic variation may comprise a deletion, substitution or insertion of one or more nucleotides. In one aspect the genetic variations to be genotyped according to the present methods comprise SNPs.

Typically the individual is a human.

The invention further provides methods for detecting the single nucleotide polymorphism in the gene of interest. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

The efficacy of a drug is a function of both pharmacodynamic effects and pharmacokinetic effects, or bioavailability. In the present invention, patient variability in drug safety, tolerability and efficacy are discussed in terms of the genetic determinants of patient variation in drug pharmacokinetics (e.g., absorption, distribution, metabolism, and excretion), drug efficacy and tolerance, and propensity for adverse events. As described herein the present invention comprises testing an individual for at least one genetic variation or occurrence of genetic polymorphism in genes associated with the rate of metabolism, testing an individual for at least one genetic variation or occurrence of genetic polymorphism in genes associated with the efficacy of or tolerance to a particular psychiatric medication, and testing an individual for at least one genetic variation or occurrence of genetic polymorphism in genes associated or related to any adverse reaction to a particular psychiatric medication. In a preferred method, an individual is also tested to detect any genetic variation or occurrence of genetic polymorphism in genes associated with a particular indication, disease or disorder to confirm the diagnosis. Accordingly, in a more preferred embodiment, the method comprises genotyping, in parallel/sequence or independently, genetic variations in the individual to determine the risk for a particular indication, disease or disorder an individual may carry. Such genes (and polymorphisms) associated with the above are listed herein. Additional exemplary information is provided in the appendices of the present application of exemplary genetic markers that may put patients at risk for particular types of psychiatric medications.

Listed below are genes that are associated with metabolism, efficacy, adverse reactions and risk. This list is not exhaustive, but representative of possible genes for analysis.

### Metabolism

Individual variation of drug effects in humans can be attributed to many factors. Among the factors, the rate of drug metabolism has been regarded as one of most important ones. Drug metabolism also known as xenobiotic metabolism is used herein to refer to the biochemical modification of pharmaceutical substances or xenobiotics respectively by living organisms, usually through specialized enzymatic systems. Drug metabolism often converts lipophilic chemical compounds into more readily excreted hydrophilic products. The rate of metabolism determines the duration and intensity of a drug's pharmacological action. A genetic defect of enzymes involved in drug metabolism, particularly cytochrome P450 (CYP), has been believed to be one of the important causal factors of adverse drug reactions. The activity of the enzymes is diverse in individuals, and the enzymes are classified into PM (poor metabolizers) IM (intermediate metabolizers) EM (extensive metabolizers) and UM (ultrarapid metabolizers) depending on the degree of activity. Partly, the genetic polymorphism of the genes causes diverse activities of the enzymes.

Other genes implicated in drug metabolism including UDP-glucuronosyltransferase, 5,10-methylenetetrahydrofolate reductase, ATP-binding cassette (ABC) transporters, and the like.

There are multiple gene mutations for CYP causing the poor metabolizer phenotype. The occurrence of genetic polymorphism has been seen in genes for CYP1A1, CYP2A6, CYP2C9, CYP2C19, CYP2D6, CYP2E1 and CYP3A5. Others implicated in drug metabolism may include: CYP1A2, CYP1B1, CYP2B6, CYP2C8, CYP2C18, CYP2E1, CYP3A4, UGT1A1, UGT1A4, UGT1A9, UGT2B4, UGT2B7, NAT1, NAT2, EPHX1, MTHFR and ABCB1.

This variability is in part attributable to genetic differences that result in slowed or accelerated oxidation of many psychotropic drugs metabolized by the cytochrome P450 (CYP450) isoenzyme system in the liver. In particular, clinically relevant variants have been identified for the isoenzymes coded by the CYP2C9, CYP2C19 and CYP2D6 genes. While the pharmacogenetic significance of CYP2C9-deficient alleles is not as prominent in psychiatry as that of CYP2D6 and CYP2C19, it is known that the gene represents a minor metabolic pathway for some antidepressants. Therefore, polymorphisms in CYP2C9 may be important in psychiatric patients deficient for other CYP450 enzymatic activities. Some of the potential consequences of polymorphic drug metabolism are extended pharmacological effect, adverse drug reactions (ADRs), lack of prodrug activation, drug toxicity, increased or decreased effective dose, metabolism by alternative deleterious pathways and exacerbated drug-drug interactions. CYP450 isoenzymes are also involved in the metabolism of endogenous substrates, including neurotransmitter amines, and have been implicated in the pathophysiology of mood disorders. CYP2D6 activity has been associated with personality traits and CYP2C9 to MDD.

The CYP2D6 gene product metabolizes several antipsychotic (e.g., aripiprazole and risperidone) and antidepressants (e.g., duloxetine, paroxetine and venlafaxine). CYP2D6 is highly polymorphic. More than 60 alleles and more than 130 genetic variations have been described for this gene, located on chromosome 22q13. Clinically, the most significant phenotype is the null metabolizer, which has no CYP2D6 activity because it has two nonfunctional CYP2D6 alleles or is missing the gene altogether. The prevalence of null metabolizers is approximately 7% in Caucasians and 1-3% in other races. Gene duplications of CYP2D6 that may lead to an ultra-rapid metabolizer (UM) phenotype are also clinically significant. A recent worldwide study suggested that up to 40% of individuals in some North African and more than 20% in Australian populations are CYP2D6 UMs. In a 2006 US survey, the prevalence of CYP2D6 UMs was 1-2% in Caucasians and African-Americans.

CYP2C9 is located on chromosome 10q24, and its gene product is involved in the metabolism of several important psychoactive substances (e.g., fluoxetine, phenytoin, sertraline and tetrahydrocannabinol). It has been reported that CYP2C9 activity is modulated by endogenous substrates such as adrenaline and serotonin. CYP2C19 is also located on chromosome 10q24, but in linkage equilibrium with CYP2C9. Its gene product is involved in the metabolism of various antidepressants (e.g., citalopram and escitalopram). For some psychotropics, a cumulative deficit in drug metabolism resulting from multigene polymorphisms in CYP2D6, CYP2C9 and CYP2C19 may be clinically significant. For example, gene products for CYP2C19 and CYP2D6 provide joint drug-metabolism pathways for various tricyclic antidepressants (e.g., amitriptyline and imipramine). Given that CYP2D6, CYP2C9 and CYP2C19 genes are not linked physically or genetically, their polymorphisms would be expected to segregate independently in populations.

CYP1A2 metabolizes many aromatic and heterocyclic amines including clozapine and imipramine. The CYP1A2*1F allele can result in a product with higher inducibility or increased activity. See Sachse et al. (1999) Br. J. Clin. Pharmacol. 47: 445-449. CYP2C19 also metabolizes many substrates including imipramine, citalopram, and diazepam. The CYP2C19 *2A, *2B, *3, *4, *5A, *5B, *6, *7, and *8 alleles encode products with little or no activity. See Ibeanu et al. (1999) J. Pharmacol. Exp. Ther. 290: 635-640.

CYP1A1 can be associated with toxic or allergic reactions by extrahepatic generation of reactive metabolites. CYP3A4 metabolizes a variety of substrates including alprazolam.

CYP1B1 can be associated with toxic or allergic reactions by extrahepatic generation of reactive metabolites and also metabolizes steroid hormones (e. g., 17p-estradiol). Substrates for CYP2A6 and CYP2B6 include valproic acid and bupropion, respectively. Substrates for CYP2C9 include Tylenol and antabuse (disulfuram). Substrates for CYP2E1 include phenytoin and carbamazepine. Decreases in activity in one or more of the cytochrome P450 enzymes can impact one or more of the other cytochrome P450 enzymes.

Exemplary alleles (shown with *) and polymorphisms include:

C430T, A1075C, 818delA, T1076C and C1080G of the cytochrome P450 2C9 (CYP2C9), rs2613delAGA, C2850T, G3183A, C3198G, T3277C, G4042A and 4125insGTGCCCACT of the cytochrome P450 2D6 (CYP2D6), A-163C, A-3860G, G3534A and C558A of the cytochrome P450 1A2 (CYP1A2), G636A, G681A, C680T, A1G, IVS5+2T >A, T358C, G431A and C1297T of the cytochrome P450 2C19 (CYP2C19), Ile462Val of the cytochrome P450 1A1 (CYP1A1), G14690A, C3699T, G19386A, T29753C and G6986A of the cytochrome P450 3A5 (CYP3A5),
P450Gene 1A1 *1A None *2 A2455G *3 T3205C *4 C2453A 1A2 *1A None *1F-164C>A *3 G1042A 1B1 *1 None *2 R48G *3 L432V *4 N453S *11 V57C *14 E281X *18 G365W *19 P379L *20 E387K *25 R469W 2A6 *1A None *1B CYP2A7 translocated to 3′-end *2 T479A *5 *1B+G6440T 2B6 *1 *2 R22C *3 S259C *4 K262R *5 R487C *6 Q172H; K262R *7 Q172H; IQ62R; R487C 2C8 *1A None *1B-271C>A *1C-370T>G *2 I269F *3 R139K; K399R *4 I264M 2C9 *1 None *2 R144C *3 I359L Cytochrome Allele Polymorphism P450Gene *5 D360E 2C18 ml T204A m2 A460T 2C19 *1A None *1B I331V *2A Splicing defect *2B Splicing defect; E92D *3 New stop codon 636G>A *4 GTG initiation codon, 1A>G *5 (A, B) 1297C>T, amino acid change (R433W)*6 395G>A, amino acid change (R132Q)*7 IVS5+2T>A, splicing defect *8 358T>C, amino acid change (W120R) 2D6 A None *2 G161C, C2850T *2N Gene duplication *3 A2549 deletion *4 G1846A *5 Gene deletion *6 T1707 deletion *7 A2935C *8 G1758T *10 C104T 12 G124A *17 C1023T, C2850T *35 G31A 2E *1A None *1C, *1D (6 or 8 bp repeats)*2 G1132A *4 G476A *5 G (−1293) C *5 C (−1053) T 4-7 T (−333) A *7 G (−71) T *7 A (−353) G 3A4 *1A None *1B A (−392) G Cytochrome Allele Polymorphism P450Gene *2 Amino acid change (S222P)*5 Amino acid change (P218R)*6 Frameshift, 831 ins A *12 Amino acid change (L373F)*13 Amino acid change (P416L) * 15A Amino acid change (R162Q)*17 Amino acid change (F189S, decreased)*18A Amino acid change (L293P, increased) 3A5 *1A None *3 A6986G *5 T12952C *6 G14960A.

While it is well known that inter-individual variation in drug metabolism is highly dependent on inherited gene polymorphisms, the debate regarding the role of genotyping in clinical practice continues. The utility of the system described herein is to provide clinically relevant indices of drug metabolism status based on combinatorial genotypes of members of the cytochrome P450 family such as CYP2C9, CYP2C19 and CYP2D6.

UDP-glucuronosyltransferase (UGT) is an enzyme which catalyzes glucuronic acid to couple with endogenous and exogenous materials in the body. The UDP-glucuronosyltransferase generates glucuronic acid coupler of materials having toxicity such as phenol, alcohol, amine and fatty acid compound, and converts such materials into hydrophilic materials to be excreted from the body via bile or urine (Parkinson A, Toxicol Pathol., 24:48-57, 1996).

The UGT is reportedly present mainly in endoplasmic reticulum or nuclear membrane of interstitial cells, and expressed in other tissues such as the kidney and skin. The UGT enzyme can be largely classified into UGT1 and UGT2 subfamilies based on similarities between primary amino acid sequences. The human UGT1A family has nine isomers (UGT1A1, and UGT1A3 to UGT1A10). Among them, five isomers (UGT1A1, UGT1A3, UGT1A4, UGT1A6 and UGT1A9) are expressed from the liver. The UGT1A gene family has different genetic polymorphism depending on people. It is known that several types of genetic polymorphism are present with respect to UGT1A1, and UGT1A3 to UGT1A10 genes (http://galien.pha.ulaval.ca/alleles/alleles.html). The polymorphism of UGT1A genes is significantly different between races. It has been confirmed that the activity of enzymes differs depending on the polymorphism, and the polymorphism is an important factor for determining sensitivity to drug treatment. UGT1A1*6 and UGT1A1*28 are related to Gilbert Syndrome (Monaghan G, Lancet, 347:578-81, 1996). Further, various functional variants which are related to various diseases have been reported. Functional variants in the UGT1A genes include 39(TA)6>(TA)7, 211G>A, 233C>T and 686C>A of a UGT1A1 gene; 31T>C, 133C>T and 140T>C of a UGT1A3 gene; 31C>T, 142T>G and 292C>T of a UGT1A4 gene; 19T>G, 541A>G and 552A>C of a UGT1A6 gene; 387T>G, 391C>A, 392G<A, 622T>C and 701T>C of a UGT1A7 gene; and −118T9>T10, 726T>G and 766G>A of a UGT1A9 gene

Similar to the cytochrome P450 family, the 5,10-methylenetetrahydrofolate reductase (MTHFR) is a key enzyme for intracellular folate homeostasis and metabolism. Methylfolic acid, synthesized from folate by the enzyme MTHFR, is required for multiple biochemical effects in the brain. A primary role involves the synthesis of dopamine in the brain. Folic acid deficiency results in fatigue, reduced energy and depression. Low folate blood levels are correlated with depression and polymorphisms of the MTHFR gene (e.g. rs1801133) are closely associated with risk of depression.

MTHFR irreversibly reduces 5-Methyltetrahydrofolate which is used to convert homocysteine to methionine by the enzyme methione synthetase. The C677T SNP of MTHFR (rs1801133) has been associated with increased vulnerability to several conditions and symptoms including depression.

The nucleotide 677 polymorphism in the MTHFR gene has two possibilities on each copy of chromosome 1: C or T. 677C (leading to an alanine at amino acid 222); 677T (leading to a valine substitution at amino acid 222) encodes a thermolabile enzyme with reduced activity. The degree of enzyme thermolability (assessed as residual activity after heat inactivation) is much greater in T/T individuals (18-22%) compared with C/T (56%) and C/C (66-67%).

MTHFR gene polymorphisms include polymorphisms in the 5,10-methylenetetrahydrofolate reductase (MTHFR) gene, including MTHFR C677T and its association with common psychiatric symptoms including fatigue and depressed mood. These symptoms are proposed to be due to hypomethylation of enzymes which breakdown dopamine through the COMT pathway. In this model, COMT is disinhibited due to low methylation status, resulting in increased dopamine breakdown.

For unipolar depression, the MTHFR C677T polymorphism has been well described and validated.

Other genes associated with drug metabolism of psychiatric drugs will be recognized by those of skill in the art.

### Efficacy and Tolerance

The response of an individual to psychiatric medications can be predicated based on the individual's genotype at one or more polymorphisms associated with certain genes. Those genes include, for example, for anti-depressants: FK506 binding protein 5 (FKBP5), angiotensin I converting enzyme 1 (ACE), serotonin 5-hydroxytryptamine receptor 1A (HTR1A), 5-hydroxytryptamine (HTR2A), Kainac acid-type glutamate receptor KA1 (GRIK4), -protein beta 3 (GNB3 G), Corticotropin releasing hormone receptor 1 (CRHR1), dopamine receptor D2 (DRD2), solute carrier family 6 member 31 (SLC6A3), Serotonin transporter (SLC6A4), Catechol-o-methyltransferase (COMT), Monoamine oxidase A (MAOA), calcium channel, voltage-dependent, L type, alpha 1C subunit (CACNA1C), solute carrier family 1 member 1 (SLC1A1), ankyrn 3 (ANK3U), brain-derived neurotrophic factor (BDNF), and apolipoprotein E (APOE), glutamate receptor, ionotropic, N-methyl D-aspartate (GRIN) 2A; anti-psychotics: PAS domain protein 3 gene (NPAS3), the XK, Kell blood group complex subunit-related family, member 4 gene (XKR4), the tenascin-R gene (TNR), the glutamate receptor, ionotropic, AMPA4 gene (GRIA4), the glial cell line-derived neurotrophic factor receptor-alpha2 gene (GFRA2), and the NUDT9P1 pseudogene located in the chromosomal region of the serotonin receptor 7 gene (HTR7), neuregulin 1 (NRG1), adrenergic α-1A-receptor (ADRA1A), and frizzled homolog 3 (FZD3). Preferably, the genes of interest to genotype are genes that affect or alter an individuals response to psychiatric medications, particularly within determination of genetic predispositions related to common neurotransmitter pathway based polymorphisms, including serotonin, glutamate and dopamine (BDNF, COMT, DRD2, DRD3, DRD4, HTR1A, HTR2A, SLC6A2, SLC6A3, SLC6A4, TPH2). More preferably, the present category refers to genes that affect neurotransmitter modulation, for example, neurotransmitter binding, transport, release, reuptake, inhibition, antagonism, agonism, synthesis, stimulation, degradation and elimination. Other neurotransmitter pathways include acetylcholine, adenosine, GABA, norepinephrine, AMPA, cannabinoid melanocortin, NMDA, GHB, sigma, opioid, histamine, monamine, melatonin, imidazoline and orexin pathways.

Exemplary polymorphisms include:

Rs2552 or a 43 bp deletion of the promotor of the serotonin transporter (SLC6A4),
Ser9Gly of the dopamine receptor D3 (DRD3),
His452Tyr and T102C of the serotonin receptor 2A (HTR2A),

FKBP5

FKBP5 regulates the cortisol-binding affinity and nuclear translocation of the glucocorticoid receptor. FKBP5 is a glucocorticoid receptor-regulating co-chaperone of hsp-90 and plays a role in the regulation of the hypothalamic-pituitary-adrenal system and the pathophysiology of depression.

FK506 regulates glucocorticoid receptor (GR) sensitivity. When it is bound to the FKBP5 receptor complex, cortisol binds with lower affinity and nuclear translocation of the receptor is less efficient. FKBP5 expression is induced by glucocorticoid receptor activation, which provides an ultra-short feedback loop for GR-sensitivity.

Changes in the hypothalamic pituitary adrenal (HPA) system are characteristic of depression. Because the effects of glucocorticoids are mediated by the glucocorticoid receptor (GR), and GR function is impaired in major depression, due to reduced GR-mediated negative feedback on the HPA axis. Antidepressants have direct effects on the GR, leading to enhanced GR function and increased GR expression.

Polymorphisms the gene encoding this co-chaperone have been shown to associate with differential up-regulation of FKBP5 following GR activation and differences in GR sensitivity and stress hormone system regulation. Alleles associated with enhanced expression of FKBP5 following GR activation, lead to an increased GR resistance and decreased efficiency of the negative feedback of the stress hormone axis. This results in a prolongation of stress hormone system activation following exposure to stress. This dysregulated stress response might be a risk factor for stress-related psychiatric disorders.

Various studies have identified single nucleotide polymorphisms (SNPs) in the FKBP5 gene associated with response to antidepressants, and one study found an association with diagnosis of depression. Polymorphisms at the FKBP5 locus have also been associated with increased recurrence risk of depressive episodes.

In fact, the same alleles are over-represented in individuals with major depression, bipolar disorder and post-traumatic stress disorder.

Individuals homozygous for the T/T genotype at one of the markers (rs1360780) reported more depressive episodes and responded better to antidepressant treatment.

For example, Lithium may be a preferred genotype based intervention for individuals with phenomenological evidence of autonomic dysfunction who express clinically relevant variants in the serotonin transporter or FKBP5 gene

HTR1A

Quantitative genetic studies have found considerable variability in the activity of the hypothalamus pituitary adrenal (HPA) axis in response to stress. The HPA axis is regulated by a neuronal network including the amygdala, which is influenced by the effects of the −1019 G/C polymorphism in the 5-HT1A (HTR1A) gene. Reduction in postsynaptic 5-HT1A receptor binding in the amygdala is correlated with untreated panic disorder. Several single nucleotide polymorphisms have been described for 5-HT1A receptor gene. The HTR1A C(−1019)G polymorphism is located in a transcriptional regulatory region and G allele and/or G/G of HTR1A C(−1019)G polymorphism genotype was found to be associated with major depression, anxiety and suicide risk.

NPY

Anxiety is integrated in the amygdaloid nuclei and involves the interplay of the amygdala and various other areas of the brain. Neuropeptides play a critical role in regulating this process. Neuropeptide Y (NPY), a 36 amino acid peptide, is highly expressed in the amygdala. It exerts potent anxiolytic effects through cognate postsynaptic Y1 receptors, but augments anxiety through presynaptic Y2 receptors.

The activity of NPY is likely mediated by the presynaptic inhibition of GABA and/or NPY release from interneurons and/or efferent projection neurons of the basolateral and central amygdala. A less active NPY rs16147-399C allele conferred slow response after 2 weeks and failure to achieve remission after four weeks of treatment. The rs16147 C allele was further associated with stronger bilateral amygdala activation in response to threatening faces in an allele-dose fashion.

A polymorphism in the upstream regulatory site for the SERT gene (SLC6A4) has been widely studied. This SERT polymorphism (serotonin transporter linked polymorphic region; 5-HTTLPR) involves the presence or absence of a 43 base-pair segment in the promoter region of the gene, which produces a long (L) or short (S) allele; a difference that can influence transcriptional activity (Heils A, Mossner R, Lesch K P. The human serotonin transporter gene polymorphism—basic research and clinical implication. J Neural Transm. 1997; 104:1005-14.; Lesch K P. Serotonin transporter and psychiatric disorders: listening to the gene. Neuroscientist. 1998; 4:25-34.). 5-HTTLPR has been associated with susceptibility to depression (Caspi et al 2003), although there is considerable heterogeneity between studies (Lotrich F E, Pollock B G, Ferrell R E. Polymorphism of the serotonin transporter: implications for the use of selective serotonin reuptake inhibitors. Am J Pharmacogenomics. 2001; 1:153-64.; Lotrich F E, Pollock B G. Meta-analysis of serotonin transporter polymorphisms and affective disorder. Psychiatr Genet. 2004). It has emerged that the 5-HTTLPR polymorphism not only influences antidepressant response to SSRI but also tolerability (Kato M, Serretti A. 2010. Review and meta-analysis of antidepressant pharmacogenetic findings in major depressive disorder. Mol Psychiatry 15:473-500). However, because of the similar redundancy of these repeats, it is often difficult to separate the two polymorphisms.

COMT

COMT is an enzyme involved in the degradation of dopamine, predominantly in the frontal cortex. Several polymorphisms in the COMT gene have been associated with poor cognition, diminished working memory, and increased anxiety as a consequence of altered dopamine catabolism. Suitable COMT gene polymorphisms include the functional common polymorphism (Val(158)Met; rs4680) that affects prefrontal function and working memory capacity and has also been associated with anxiety and emotional dysregulation.

The COMT rs4680 G/G genotype (Val/Val homozygous genotype) confers a significant risk of worse response after 4-6 weeks of antidepressant treatment in patients with major depression. There is a negative influence of the higher activity COMT rs4680rs4680 G/G genotype on antidepressant treatment response during the first 6 weeks of pharmacological treatment in major depression, possibly conferred by decreased dopamine availability. This finding suggests a potentially beneficial effect of interventions such as transcranial magnetic stimulation, which has been shown to increase metabolic activity in the dorsolateral prefrontal cortex in a genotype specific manner. Conversely, COMT Met/Met variants may have an opposite phenotype and cluster of symptoms including increased vulnerability to addiction. Treatments which could potentially address these variants include S-adenosyl methionine (a COMT agonist which may lower prefrontal dopamine) or a dopamine antagonist.

Polymorphisms for COMT also include Catechol-o-COMT G158A (Also known as Val/Met) methyltransferase G214 T A72S G101C C34S G473A.

SLC6A4

The S allele has also been associated with diminished response to several SSRIs as compared with the L allele in multiple studies (Arias B, Gasto C, Catalan R, et al. Variation in the serotonin transporter gene and clinical response to citalopram in major depression. Am J Med Genet. 2000; 96:536.; Pollock B G, Ferrell R E, Mulsant B H, et al. Allelic variation in the serotonin transporter promoter affects onset of paroxetine treatment response in late-life depression. Neuropsychopharmacology. 2000; 23:587-90.; Zanardi R, Benedetti F, Di Bella D, et al. Efficacy of paroxetine in depression is influenced by a functional polymorphism within the promoter of the serotonin transporter gene. J Clin Psychopharmacol. 2000; 20:105-6.; Rausch J L, Johnson M E, Fei Y-J, et al. Initial conditions of serotonin transporter kinetics and genotype: influence on SSRI treatment trial outcome. Biol Psychiatry. 2002; 51:723-32.; Yu Y-Y, Tsai S-J, Chen T-J, et al. Association study of the serotonin transporter promoter polymorphism and symptomatology and antidepressant response in major depressive disorders. Mol Psychiatry. 2002; 7:1115-19.; Arias B, Catalan R, Gasto C, et al. 5-HTTLPR polymorphism of the serotonin transporter gene predicts non-remission in major depression patients treated with citalopram in a 12-weeks follow up study. J Clin Psychopharmacol. 2003; 23:563-7.), although there are two exceptions in Asian populations (Kim D K, Lim S-W, Lee S, et al. Serotonin transporter gene polymorphism and antidepressant response. Neuroreport. 2000; 11:215-19., Ito K, Yoshida K, Sato K, et al. A variable number of tandem repeats in the serotonin transporter gene does not affect the antidepressant response to fluvoxamine. Psychiatry Res. 2002; 111:235-9.). The S allele may also increase vulnerability to SSRI side effects (Mundo E, Walker M, Cate T, et al. The role of serotonin transporter protein gene in antidepressant-induced mania in bipolar disorder: preliminary findings. Arch Gen Psychiatry. 2001; 58:539-44.; Murphy G M, Kremer C, Rodrigues H, et al. The apolipoprotein E epsilon4 allele and antidepressant efficacy in cognitively intact elderly depressed patients. Biol Psychiatry. 2003a; 54:665-73.). While the general finding of worse outcome in SSRI-treated patients with the S allele has been well replicated, discrepant reporting in several of these studies makes it difficult to determine the effect size of this polymorphism. Among issues to be further clarified is the effect of 5-HTTLPR in different ethnic populations; linkage disequilibrium with other polymorphisms in different ethnic populations; the effect size in different age groups and at different doses of SSRIs; delineating which depressive symptoms and side effects are influenced; and determining how this polymorphism interacts with other polymorphisms. Moreover, the role of other SLC6A4 polymorphisms remains comparatively unexamined (Lesch 1998; Battersby S, Ogilvie A D, Blackwood D H R, et al. Presence of multiple functional polyadenylation signals and a single nucleotide polymorphism in the 3′untranslated region of the human serotonin transporter gene. J Neurochem. 1999; 72:1384-8.; Michaelovsky E, Frisch A, Rockah R, et al. A novel allele in the promoter region of the human serotonin transporter gene. Mol Psychiatry. 1999; 4:97-9.; M. Nakamura, S. Ueno, A. Sano & H. Tanabe (2000). “The human serotonin transporter gene linked polymorphism (5-HTTLPR) shows ten novel allelic variants”. Molecular Psychiatry 5 (1): 32-38.; Ito et al 2002).

Although researchers commonly report the polymorphism with two variations: a short (“S”) and a long (“L”), it can be subdivided further. One such study found 14 different alleles were found in different populations [M. Nakamura, S. Ueno, A. Sano & H. Tanabe (2000). “The human serotonin transporter gene linked polymorphism (5-HTTLPR) shows ten novel allelic variants”. Molecular Psychiatry 5 (1): 32-38] In connection with the region are two single nucleotide polymorphisms (SNP) which contribute to this subdivision: rs25531 and rs25532. [L. Murphy & Klaus-Peter Lesch (February 2008). “Targeting the murine serotonin transporter: insights into human neurobiology”. Nature Reviews Neuroscience 9 (2): 85-86].

With the results from one study the polymorphism was thought to be related to treatment response so that long-allele patients respond better to antidepressants [L. Kathryn Durham, Suzin M. Webb, Patrice M. Milos, Cathryn M. Clary, Albert B. Seymour (August 2004). “The serotonin transporter polymorphism, 5HTTLPR, is associated with a faster response time to sertraline in an elderly population with major depressive disorder”. Psychopharmacology 174 (4): 525-529] Another antidepressant treatment response study did, however, rather point to the rs25531 SNP, [Jeffrey B. Kraft, Susan L. Slager, Patrick J. McGrath & Steven P. Hamilton (September 2005). “Sequence analysis of the serotonin transporter and associations with antidepressant response”. Biological psychiatry 58 (5): 374-381] and a large study by the group of investigators found a “lack of association between response to an SSRI and variation at the SLC6A4 locus”. [Jeffrey B. Kraft, Eric J. Peters, Susan L. Slager, Greg D. Jenkins, Megan S. Reinalda, Patrick J. McGrath & Steven P. Hamilton (March 2007). “Analysis of association between the serotonin transporter and antidepressant response in a large clinical sample”. Biological Psychiatry 61(6): 734-742].

Other serotonin related genes and polymorphisms include Serotonin Transporter 5-HTTR Promoter repeat (44 bp insertion (L) /deletion (S) (L=Long form; S=Short form) Exon 2 variable repeat A1815C G603C G167C Serotonin Receptor 1A HTR1A RsaI G815A, G272D G656T, R219L C548T, P551L A82G, 128V G64A, G22S C47T, P16L Serotonin Receptor 1B HTR1B G861C G861C, V287V T371G, F124C T655C, F219L A1099G, 1367V G1120A E374K Serotonin Receptor 1D HTR1D G506T C173T C794T, S265L Serotonin Receptor 2A HTR2A C74A T102C T516C C1340T C1354T Serotonin Receptor 2C HTR2C G796C C10G, L4V G68C, C23S

DRD2

Several lines of evidence suggest that antipsychotic drug efficacy is mediated by dopamine type 2 (D(2)) receptor blockade. Six studies reported results for the −141 C Ins/Del polymorphism (rs1799732) which indicated that the Del allele carrier is significantly associated with poorer antipsychotic drug response relative to the Ins/Ins genotype. These findings suggest that variation in the D(2) receptor gene can, in part, explain variation in the timing of clinical response to antipsychotics and higher risk of weight gain in deletion allele subtypes of the DRD2 gene.

Other dopamine related genes (and polymorphisms) include Dopamine Transporter DAT1, 40 bp VNTR SLC6A3 10 repeat allele G710A, Q237R C124T, L42F Dopamine Receptor D1 DRD1 DRD1 B2 T244G C179T G127A TUG C81T T595G, S199A G150T, R505 C110G, T37R A109C, T37P Dopamine Receptor D2 DRD2 TaqI A A1051G, T35A C932G, S311C C928, P310S G460A, V1541 Dopamine Receptor D3 DRD3 Ball in exon I MspI DRD3 1 Gly/Ser (allele 2) A25G, S9G Dopamine Receptor D4 DRD4 48 repeat in exon 3 7 repeat allele 12/13 bp insertion/deletion T581G, V194G C841G, P281A Dopamine Receptor D5 DRD5 T978C L88F A889C, T297P G1252A, V418I G181A, V61M G185C, C625 T263G, R88L G1354A, W455.

CACNA1C

The calcium ion is one of the most versatile, ancient, and universal of biological signaling molecules, known to regulate physiological systems at every level from membrane potential and ion transporters to kinases and transcription factors. Disruptions of intracellular calcium homeostasis underlie a host of emerging diseases, the calciumopathies. Cytosolic calcium signals originate either as extracellular calcium enters through plasma membrane ion channels or from the release of an intracellular store in the endoplasmic reticulum (ER) via inositol triphosphate receptor and ryanodine receptor channels. Therefore, to a large extent, calciumopathies represent a subset of the channelopathies, but include regulatory pathways and the mitochondria, the major intracellular calcium repository that dynamically participates with the ER stores in calcium signaling, thereby integrating cellular energy metabolism into these pathways, a process of emerging importance in the analysis of the neurodegenerative and neuropsychiatric diseases.

Molecular genetic analysis offers opportunities to advance our understanding of the nosological relationship between psychiatric diagnostic categories in general and the mood and psychotic disorders in particular. The CACNA1C gene encodes one subunit of a calcium channel. Results suggest that ion channelopathies may be involved in the pathogenesis of bipolar disorder, schizophrenia and autism with an overlap in their pathogenesis based upon disturbances in brain calcium channels.

CACNA1C encodes for the voltage-dependent calcium channel L-type, alpha 1c subunit. Gene variants in CACNA1 (e.g. rs1006737) are associated with altered calcium gating and excessive neuronal depolarization. CACNA1 polymorphisms have been associated with increased risk of bipolar disease and schizophrenia.

Psychiatric disease phenotypes, such as schizophrenia, bipolar disease, recurrent depression and autism, produce a constitutionally hyperexcitable neuronal state that is susceptible to periodic decompensations. The gene families and genetic lesions underlying these disorders may converge on CACNA1C, which encodes the voltage gated calcium channel.

These findings suggest some degree of overlap in the biological underpinnings of susceptibility to mental illness across the clinical spectrum of mood and psychotic disorders, and show that at least some loci can have a relatively general effect on susceptibility to diagnostic categories based upon alterations in calcium signaling. Abnormalities in synaptic pathways can also be probed by specific brain imaging modalities which probe the integrity of axons and white matter. For instance, diffusion tensor imaging demonstrated decreased white matter integrity, indicated by lower fractional anisotropy and longitudinal diffusivity, in the ANK3 rs10994336 risk genotype in the anterior limb of the internal capsule and carriers of the A allele of the CACNA1C gene showed significantly increased gray matter volume and reduced functional connectivity within a corticolimbic frontotemporal regions, supporting the effects of the rs1006737 on frontotemporal networks, This suggests that influence of CACNA1C variation on corticolimbic functional connectivity.

Medical interventions which address heightened neuronal depolarization in the hippocampus in association with calcium channel variants should be considered.

Agents which modulate or exert effects on calcium channels may be preferred agents to use in patients with psychiatric disorders in patients who exhibit these variants, as will be further described in subsequent paragraphs. Such agents may include specific L-type voltage-gated calcium channel inhibitors such as Nimodipine, Flunarizine and the like. They may also include other mood stabilizers, such as Lithium or Valproic acid.

ANK3

Another biomarker includes the ANK3 gene (e.g. rs10994336). Genetic variants in ankyrin 3 (ANK3) have recently been shown to be associated with bipolar disorder and schizophrenia. The gene ANK3 encodes ankyrin-G, a large protein whose neural-specific isoforms, localized at the axonal initial segment and nodes of Ranvier, may help maintain ion channels and cell adhesion molecules. ANK3 is essential for both normal clustering of voltage-gated sodium channels at axon initial segments. Personalized treatments for individuals with this variant may include sodium channel modulating agents, such as Lamotrigine.

In patients with sodium channel gene variants, there may be altered expression of depolarization across the axon which is effecting normal neural conduction. This may provide a model of how the oscillation between long term depression and potentiation becomes abnormal (e.g., an imbalance between LTP and LTD). The sodium channels may then dis-regulate the sodium channels. This bipolar model is represents dis-regulation between LTP and LTD, and may result from the sodium channel variation. In patients with oscillatory affective states secondary to normal axonal propagation, sodium channel blockers may be recommended. Lamotrigine (or other sodium channel blocking drugs) may be used if there is a polymorphism in the ANK3 gene.

BDNF

Brain-derived neurotrophic factor is a member of the nerve growth factor family. It is induced by cortical neurons and is necessary neurogenesis and neuronal plasticity. BDNF has been shown to mediate the effects of repeated stress exposure and long term antidepressant treatment on neurogenesis and neuronal survival within the hippocampus. The BDNF Val66Met variant is associated with hippocampal dysfunction, anxiety, and depressive traits. Previous genetic work has identified a potential association between a Val66Met polymorphism in the BDNF gene and bipolar disorder. Meta-analysis based on all original published association studies between the Val66Met polymorphism and bipolar disorder up to May 2007 shows modest but statistically significant evidence for the association between the Val66Met polymorphism and bipolar disorder from 14 studies consisting of 4248 cases, 7080 control subjects and 858 nuclear families.

The BDNF gene may play a role in the regulation of stress response and in the biology of depression and the expression of brain-derived neurotrophic factor (BDNF) may be a downstream target of various antidepressants.

Exposure to stress causes dysfunctions in circuits connecting hippocampus and prefrontal cortex. BDNF is down-regulated after stress. Acute treatment with the antidepressant tianeptine reverses stress-induced down-regulation of BDNF. Tianeptine increases the phosphorylation of Ser831-GluA1. Psychological stress down-regulates a putative BDNF signaling cascade in the frontal cortex in a manner that is reversible by the antidepressant tianeptine. Thus agents which promote BDNF are novel mechanisms to treat stress induced alterations in the limbic system

Activation of AMPA receptors by agonists is thought to lead to a conformational change in the receptor causing rapid opening of the ion channel, which stimulates the phosphorylation of CAMK11/PKC sites and subsequently enhance BDNF expression.

A structural class of AMPA receptor positive modulators derived from aniracetam are called Ampakines Aniracetam and Nefiracetam are neurological agents called ‘racetams’ that are analogs of piracetam. They are regarded as AMPA receptor potentiators and CaMKII agonists.

Small molecules that potentiate AMPA receptor show promise in the treatment of depression, a mechanism which also appears to be mediated by promoting BDNF via CaMKII pathways. Depression is associated with abnormal neuronal plasticity. AMPA receptors mediate transmission and plasticity at excitatory synapses in a manner which is positively regulated by phosphorylation at Ser831-GluR1, a CaMKII/PKC site.

Aniracetam [1-(4-methoxybenzoyl)-2-pyrrolidinone] is an AMPA receptor potentiator that preferentially slows AMPA receptor deactivation. AMPA receptor potentiators (ARPs), including aniracetam, exhibit antidepressant-like activity in preclinical tests. Unlike most currently used antidepressants, interactions of aniracetam with proteins implicated in AMPA receptor trafficking and with scaffolding proteins appear to account for the enhanced membrane expression of AMPA receptors in the hippocampus after antidepressant treatment. The signal transduction and molecular mechanisms underlying alpha-amino-3-hydroxy-5-methyl-4-isoxazole propionate (AMPA)-mediated neuroprotection evokes an accumulation of BDNF and enhance TrkB-tyrosine phosphorylation following the release of BDNF. AMPA also activate the downstream target of the phosphatidylinositol 3-kinase (PI3-K) pathway, Akt. The increase in BDNF gene expression appeared to be the downstream target of the PI3-K-dependent by AMPA agonists and Tianeptine (described below). Thus, AMPA receptors protect neurons through a mechanism involving BDNF release, TrkB receptor activation, and up-regulation of CaMKII which increase BDNF expression.

Olfactory bulbectomized (OBX) mice exhibit depressive-like behaviors. Chronic administration (1 mg/kg/day) of nefiracetam, a prototype cognitive enhancer, significantly improves depressive-like behaviors. Decreased calcium/calmoculin-dependent protein kinase II mediates the impairment of hippocampal long-term potentiation in the olfactory bulbectomized mice. Nefiracetam treatment (1 mg/kg/day) significantly elevated CaMKII in the amygdala, prefrontal cortex and hippocampal CA1 regions. Thus, CaMKII, activation mediated by nefiracetam treatment elicits an anti-depressive and cognition-enhancing outcome.

SCN1A

A polymorphism within SCN1A (encoding the 1 subunit of the type I voltage-gated sodium channel) has been replicated in three independent populations of 1699 individuals. Functional magnetic resonance imaging during working memory task detected SCN1A allele-dependent activation differences in brain regions typically involved in working memory processes. These results suggest an important role for SCN1A in human short-term memory.

Voltage-gated sodium channels have an important role in the generation and propagation of the action potential and consist of an alpha subunit, which forms the ion conduction pore, and two auxiliary beta subunits. The alpha subunit has four homologous domains and different genes (SCN1A through SCN11A) encode different alpha subunits named Nav1.1 through Nav1.9 The SCN1A is expressed in brain regions critical for memory formation, regulates excitability of neuronal membranes and several SCN1A mutations are known to cause a variety of neurological diseases such as familial hemiplegic migraine. Some antiepileptic drugs, such as phenytoin and carbamazepine, bind to voltage-gated sodium channels and genetic variability within SCN1A may predict the response to carbamazepine and phenytoin in patients diagnosed with epilepsy.

Lamotrigine, another antiepileptic drug that binds to voltage-gated sodium channels, is an effective maintenance treatment for bipolar disorder, particularly for prophylaxis of depression, a mental disorder with commonly observed working memory deficits. A recent fMRI study reports that lamotrigine treatment in depressed patients results in increased activation of brain regions typically involved in working memory processes.

Heterozygous individuals of the SCN1A gene (rs10930201) showed significantly increased brain activations compared with homozygous A allele carriers in the right superior frontal gyrus/sulcus, indicating a potential biomarker for Lamotrigine in these individuals with mood disorder.

HTR2A

HTR2A encodes the serotonin 2A receptor, which is down-regulated by citalopram. HTR2A also is known as HTR2 and 5-HT2A receptor. HTR2A is located on chromosome 13q14-q21. HTR2A is identified by GenBank Accession Number NM-000621.

Seven distinct 5-HT receptors have been identified (5-HT1-7). The 5HT2A, B, and C subtypes are positively coupled with the enzyme phospholipase C (PLC). The 5-HT2A receptors are postsynaptic receptors that are highly enriched in neocortex and regulate the function of prefrontal-subcortical circuits. The 5-HT2A receptors interact with Gq/G11 guanine nucleotide binding proteins (G proteins) and thereby stimulate PLC to produce the intracellular second messengers sn-1,2-DAG (an endogenous activator of protein kinase C) and inositol-1,4,5-triphosphate (IP3), which stimulates the release of Ca++ from intracellular stores. The markers in HTR2A associated with treatment outcome include rs7997012, rs1928040, and rs7333412. Other markers in HTR2A that correlate with treatment outcome include rs977003; rs1745837; and rs594242.

GRIK4

GRIK4 encodes a subunit of a kainate glutamate receptor. GRIK4 also is known as KA1, EAA1, and GRIK. GRIK4 is located on chromosome 11q22.3. GRIK4 is identified by GenBank Accession Number NM-014619. GRIK4 encodes a protein that belongs to the glutamate-gated ionic channel family. Glutamate functions as the major excitatory neurotransmitter in the central nervous system through activation of ligand-gated ion channels and G protein-coupled membrane receptors. The protein encoded by GRIK4 forms functional heteromeric kainate-preferring ionic channels with the subunits encoded by related gene family members.

The polymorphism that is associated with the outcome of treatment with antidepressant medication (e.g., a decreased risk of non-response to treatment with antidepressant medication) in the GRIK4 gene typically is within intron 1 of GRIK4 (GenBank Accession Number NM-000828). In such a situation, intron 1 of GRIK4 contains cytosine at position 201, rather than thymine. The marker in GRIK4 associated with the outcome of treatment with antidepressant medication is rs1954787. Other markers in GRIK4 that correlate with treatment outcome include rs6589832; rs3133855; rs949298; rs2156762; rs948028; rs2186699; and rs607800.

BCL2

BCL2 encodes a protein involved in cellular development and survival and may be involved in neurogenesis. BCL2 is also known as bcl-2 and resides on chromosome 18q22. BCL2 is identified by GenBank Accession Numbers NM-000633.2 and NM-000657.2. The polymorphism that is associated with the outcome of treatment with antidepressant medication (e.g., that correlates a decreased risk of non-response to treatment with antidepressant medication) is typically in intron 2 of BCL2. In such a situation, intron 2 of BCL2 typically contains cytosine at position 201, rather than adenine.

The markers in BCL2 that correlate with treatment outcome include rs4987825; rs4941185; rs1531695; and rs2850763.

Other markers include:

| Gene | Symbol | Polymorphism |
|---|---|---|
| Dopamine Transporter | DATI, | 40 bp VNTR |
| | SLC6A3 | 10 repeat allele |
| | | G710A, Q237R |
| | | C124T, L42F |
| Dopamine Receptor D1 | DRDI | DRD 1 B2 |
| | | T244G |
| | | C179T |
| | | G127A |
| | | T11G |
| | | C81T |
| | | T5950, S199A |
| | | G150T, R50S |
| | | C1100, T37R |
| | | AI09C, T37P |
| Dopamine Receptor D2 | DRD2 | TaqI A |
| | | AI051G, T35A |
| | | C932G, S311 C |
| | | C928, P31 OS |
| | | G460A, V1541 |
| Dopamine Receptor D3 | DRD3 | Ball in exon I |
| | | MspI |
| | | DRD31 |
| | | Gly/Ser (allele 2) |
| | | A250, S9G |
| Dopamine Receptor D4 | DRD4 | 48 repeat in exon 3 |
| | | 7 repeat allele. |
| | | 12/13 bp insertion/deletion |
| | | T581G, V194G |
| | | C841G, P281A |
| Dopamine Receptor D5 | DRD5 | T978C |
| | | L88F |
| | | A889C, T297P |
| | | G1252A, V4181 |
| | | G181A, V61M |
| | | G185C, C62S |
| | | T2630, R88L |
| | | G1354A, W455 |
| Tryptophan | TPH | A218C |
| Hydroxylase | | A779C |
| | | G-5806T |
| | | A-6526G |
| | | (CT)m(CAMCT)p allele 194 |
| | | in 3′ UTR, 5657 bp distant |
| | | from exon 11 |
| Serotonin Transporter | 5-HTTR | Promoter repeat (44bp |
| | | insertion (L)/deletion(S) (L = |
| | | Long form; S = ShOli form) |
| | | Exon 2 variable |
| | | repeat |
| | | A1815C |
| | | G603C |
| | | G167C |
| Serotonin Receptor 1A | HTR1A | RsaI |
| | | G815A, G272D |
| | | G656T, R219L |
| | | G548T, P551L |
| | | A82G, 128V |
| | | G64A, |
| | | G22S |
| | | C47T, P16L |
| Serotonin Receptor 1B | HTR1B | G861C |
| | | G861C, V287V |
| | | T371G, F124C |
| | | T655C, F219L |
| | | A1 099G, I367V |
| | | G1120A, E374K |
| Serotonin Receptor 1D | HTR1D | G506T |
| | | C173T |
| | | C794T, S265L |
| Serotonin Receptor 2A | HTR2A | C74A |
| | | T102C |
| | | T516C |
| | | C1340T |
| | | C1354T |
| Serotonin Receptor 2C | HTR2C | G796C |
| | | C1OG, L4V |
| | | G68C, C23S |
| Catechol-o-methyltransferase | COMT | G158A (Also known |
| | G214T | as Val/Met) |
| | A72S | |
| | G101C | |
| | C34S | |
| | G473A | |
| | ARVCF | rs165599 |

More genes affecting efficacy: ABCB1, ADM, SBF2, AKT1, ARVCF, COMT, BDNF, CACNA1C, CACNG2, CNTF, CREB1, FAM119A, DRD3, DRD4, DTNBP1, FKBP5, GRIA2, GRIK4, GRM3, GSK3B, HTR1A, NR3C1, NTRK2, OPRM1, RGS4, SERPINE1, TPH2, SLC6A2, SLC6A3, ZBTB42, and CREB1.

### Side Effects/Adverse Effect

In a large patient population, a medication that is proven efficacious in many patients often fails to work in some other patients. Furthermore, when it does work, it may cause serious side effects, even death, in a small number of patients. Adverse drug reactions are a principal cause of the low success rate of drug development programs (less than one in four compounds that enters human clinical testing is ultimately approved for use by the U.S. Food and Drug Administration (FDA)). Adverse drug reactions are generally undesired effects, e.g., side effects, that can be categorized as 1) mechanism based reactions and 2) idiosyncratic, “unpredictable” effects apparently unrelated to the primary pharmacologic action of the compound. Although some side effects appear shortly after administration, in some instances side effects appear only after a latent period. Adverse drug reactions can also be categorized into reversible and irreversible effects. The methods of this invention are useful for identifying the genetic basis of both mechanism based and ‘idiosyncratic’ toxic effects, whether reversible or not. Methods for identifying the genetic sources of interpatient variation in efficacy and mechanism based toxicity may be initially directed to analysis of genes affecting pharmacokinetic parameters, while the genetic causes of idiosyncratic adverse drug reactions are more likely to be attributable to genes affecting variation in pharmacodynamic responses or immunological responsiveness.

A 1998 meta-analysis of 39 prospective studies in US hospitals estimated that 106,000 Americans die annually from ADRs. Adverse drug events are also common (50 per 1000 person years) among ambulatory patients, particularly the elderly on multiple medications. The 38% of events classified as ‘serious’ are also the most preventable. It is now clear that virtually every pathway of drug metabolism, transport and action is susceptible to gene variation. Within the top 200 selling prescription drugs, 59% of the 27 most frequently cited in ADR studies are metabolized by at least one enzyme known to have gene variants that code for reduced or nonfunctional proteins.

A number of compounds are associated with adverse effects that may manifest greater in those individuals showing certain genetic variability. In a particular aspect of the present invention, the invention comprises genotyping genes that increase or decrease for drug hypersensitivity in individuals, including TNFalpha (TNFa) gene, MICA, MICB, and/or HLA genes.

### TNFalpha

The immunologic effector molecule Tumor Necrosis Factor alpha (TNFa) is known to be polymorphic, and a number of polymorphisms have been reported in the TNFa promoter region. Some reports indicate that such promoter polymorphisms influence immunologic disease (Bouma et al. , Scand. J. Immunol. 43: 456 (1996); Allen et al. , Mol. Immunology 36: 1017 (1999)), whereas others suggest that observed associations between TNFa polymorphisms and disease occurrence are not due to functional effects of TNFa, but due to the linkage disequilibrium of TNFa with selectable HLA alleles (Uglialoro et al., Tissue Antigens, 52: 359 (1998)). A list of TNFa promoter polymorphisms is provided by Allen et al. , Mol. Immunology 36: 1017 (1999). Due to variation in reported sequences and numbering, the G (−237) A polymorphism has also been referred to as G-238A, and the G (−308) A polymorphism is located at the −307 position on the above sequence. A further polymorphism, C (−5,100) G, investigated in the present research was an C/G polymorphism in the 5′untranslated region of TNFa.

A number of the TNFa promoter polymorphisms observed to date are G/A polymorphisms clustered in the region of −375 to −162 bp; that some of these polymorphisms lie within a common motif; and suggest that the motif could be a consensus binding site for a transcriptional regulator or might influence DNA structure. The G/A polymorphism at −237 has been reported to affect DNA curvature (D'Alfonso et al., Immunogenetics 39: 150 (1994)). Huizinga et al. (J. Neuroimmunology 72: 149, 1997) reported significantly less TNFa production by LPS-stimulated cells from individuals heterozygous (G/A) at −237 (compared to G/G individuals); however, a separate study did not observe these effects (Pociot et al. , Scand. J. Immunol. 42: 501, 1995). The G (−237) A polymorphism has also been reported to affect autoimmune disease (Brinkman et al. , Br. J. Rheumatol. 36: 516 1997 (rheumatoid arthritis); Huizinga et al. , J. Neuroimmunology 72: 149 1997 (multiple sclerosis); Vinasco et al., Tissue Antigens, 49: 74 1997 (rheumatoid arthritis)) and infectious disease (Hohler et al. , Clin. Exp. Immunol. 111: 579 1998 (hepatitis B); Hohler et al. , J. Med. Virol. 54: 173 1998 (hepatitis c)).

As is well known genetics, nucleotide and amino acid sequences obtained from different sources for the same gene may vary both in the numbering scheme and in the precise sequence. Such differences may be due to inherent sequence variability within the gene and/or to sequencing errors. Accordingly, reference herein to a particular polymorphic site by number (e. g., TNFa G-238A) will be understood by those of skill in the art to include those polymorphic sites that correspond in sequence and location within the gene, even where different numbering/nomenclature schemes are used to describe them.

### HLA

The HLA complex of humans (major histocompatibility complex or MHC) is a cluster of linked genes located on chromosome 6. (The TNFa and HLA B loci are in proximity on chromosome 6). The HLA complex is classically divided into three regions: class I, II, and III regions (Klein J. In: Gotze D, ed. The Major Histocompatibility System in Man and Animals, New York: Springer-Verlag, 1976: 339-378). Class I HLAs comprise the transmembrane protein (heavy chain) and a molecule of beta-2 microglobulin. The class I transmembrane proteins are encoded by the HLA-A, HLA-B and HLA-C loci. The function of class I HLA molecules is to present antigenic peptides (including viral protein antigens) to T cells. Three isoforms of class II MHC molecules, denoted HLA-DR, -DQ, and -DP are recognized. The MHC class II molecules are heterodimers composed of an alpha chain and a beta chain; different alpha- and beta-chains are encoded by subsets of A genes and B genes, respectively. Various HLA-DR haplotypes have been recognized, and differ in the organization and number of DRB genes present on each DR haplotype; multiple DRB genes have been described. Bodmer et al., Eur. J. Immunogenetics 24: 105 (1997); Andersson, Frontiers in Bioscience 3: 739 (1998).

The MHC exhibits high polymorphism; more than 200 genotypical alleles of HLA-B have been reported. See e. g. , Schreuder et al. , Human Immunology 60: 1157-1181 (1999); Bodmer et al. , European Journal of Immunogenetics 26: 81-116 (1999). Despite the number of alleles at the HLA-A, HLA-B and HLA-C loci, the number of haplotypes observed in populations is smaller than mathematically expected. Certain alleles tend to occur together on the same haplotype, rather than randomly segregating.

This is called linkage disequilibrium (LD) and may be quantitated by methods as are known in the art (see, e. g. , Devlin and Risch, Genomics 29: 311(1995); B S Weir, Genetic Data Analysis II, Sinauer Associates, Sunderland, Md. (1996)). “Linkage disequilibrium” refers to the tendency of specific alleles at different genomic locations to occur together more frequently than would be expected by chance.

Assessing the risk of a patient for developing an adverse drug reaction in response to a drug, can be accomplished by determining the presence of an HLA genotypes including HLA-B allele selected from the group consisting of HLA-B*1502, HLA-B*5701, HLA-B*5801 and HLA-B*4601, wherein the presence of the HLA-B allele is indicative of a risk for an adverse drug reaction. Other drugs include carbazapine, oxcarbazepine, licarbazepine, allopurinol, oxypurinol, phenytoin, sulfasalazine, amoxicillin, ibuprofen, and ketoprofen. Other subtypes of HLA-B15, B58 or B46, such as HLA-B*1503 or *1558, can also be used to predict the risk for developing an ADR.

More specifically, HLA-B* 1502 being associated with carbamazepine-specific severe cutaneous reactions and other forms of hypersensitivity, HLA-B*5701 being associated with abacavir hypersensitivity, HLA-B*5801 being associated with allopurinol-induced severe cutaneous adverse reactions, HLA-A29, -B 12, -DR7 being associated with sulfonamide-SJS, HLA-A2, B 12 being associated with oxicam-SJS, HLA-B59 being associated with methazolamide-SJS, HLA-Aw33, B17/Bw58 being associated with allopurinol-drug eruption, HLA-B27 being associated with levamisole-agranulocytosis, HLA-DR4 being associated with hydralazine-SLE , HLA-DR3 being associated with penicillamine toxicity, HLA-B38, DR4, DQw3 being associated with clozapine-agranulocytosis, HLA-A24, B7, DQwI being associated with dipyrone-agranulocytosis. Preferably, the HLA genotype is selected from the group consisting of HLA-B* 1502 being associated with carbamazepine-specific severe cutaneous reactions and other forms of hypersensitivity, HLA-B*5701 with abacavir hypersensitivity and HLA-B*5801 with allopurinol-induced severe cutaneous adverse reactions, and preferably being HLA-B* 1502.

MICA and MICB

The MHC (HLA) class I chain-related gene A (MICA) and MHC (HLA) class I chain-related gene B (MICB) belong to a multicopy gene family located in the major histocompatibility complex (MHC) class I region near the HLA-B gene. They are located within a linkage region on chromosome 6p around HLA-B and TNFalpha. The encoded MHC class I molecules are induced by stress factors such as infection and heat shock, and are expressed on gastrointestinal epithelium.

MICA is reported as highly polymorphic. The occurrence of MICA single nucleotide polymorphisms in various ethnic groups is reported by Powell et al., Mutation Research 432: 47 (2001). Polymorphisms in MICA have been reported to be associated with various diseases, although in some cases the association was attributable to linkage disequilibrium with HLA genes. See, e. g. , Salvarani et al. J Rheumatol 28: 1867 (2001); Gonzalez et al., Hum Immunol 62: 632 (2001); Seki et al., Tissue Antigens 58: 71 (2001).

Various polymorphic forms of MICB have been reported (see, e. g., Visser et al., Tissue Antigens 51: 649 (1998); Kimura et al., Hum Immunol 59: 500 (1998); Ando et al., Immunogenetics 46: 499 (1997); Fischer et al. , Eur J Immunogenet 26: 399 (1999)).

More genes affecting adverse reactions: ABCB1, ABCC2, ADRB3, ANKK1, ASTN2, ATF7IP2, BAT2, BAT3, BRUNOL4, CDH13, CERKL, CLCN6, MTHFR, CLMN, FHOD3, GNB3, GPR98, GRIA3, KIRREL3, LEP, LEPR, LOC729993, LTA, TNF, MC4R, MEIS2, NRG3, NUBPL, PALLD, PMCH, PPARD, PRKAA1, PRKAR2B, RNF144A, SCN1A, SLCO3A1, and SOX5.

Preferably, one or more genetic variations are evaluated in each of the categories. For example, one or more mutations, polymorphisms and/or alleles are evaluated in one or more genes in each of the categories. Preferably, one or more genetic variations, e.g., polymorphisms, are evaluated in multiple genes. For example, one or more polymorphisms may be evaluated for combinations of CYP1A2, CYP2C19, CYP2D6, and/or UGT1A4. In a more preferred method, there are two or more genetic variations genotyped in a panel, and more preferably three, four, five, six, seven, eight, nine, ten, eleven, twelve, thirteen, fourteen, fifteen or more genes in a panel.

Although the genes discussed herein are listed in separate categories for convenience in the present application, such genes may be associated in other categories. For example, genetic variations listed within the risk category may affect genes within efficacy, metabolism, and/or adverse effects. Or a gene associated with metabolism of drugs may affect efficacy (e.g., neurotransmitter activity), adverse effect and/or risk. Or a gene associated with efficacy of drugs may affect metabolism, adverse effect and/or risk. Or a gene associated with adverse effect of drugs may affect efficacy (e.g., neurotransmitter activity), metabolism and/or risk. However, generally, those of skill in the art will look at the effect of the genetic variation to determine which category a particular gene will be categorized in the present invention. For example, a serotonin receptor 2A and 2C are associated with adverse reactions to paroxetine and fluvoxamine, and atypical antipsychotic-induced weight gain and thus categorized and associated with adverse reactions/side effects, although listed herein within efficacy. Serotonin receptors and transporter genes affect the efficacy of certain drugs through different mechanisms such as transport, inhibition, agonism and the like. Similarly, although listed within genes associated with metabolism, the high carrier prevalence of deficient CYP450 alleles may expose 50% of patients to preventable severe side effects. If these patients were carriers of gene polymorphisms resulting in deficient psychotropic metabolism, their risk of adverse drug effects would substantially increase. Were DNA typing to be performed after development of drug resistance or intolerance, such information could guide subsequent pharmacotherapy and assist in diagnosing drug-induced side effects. The value of DNA typing for diagnosing severe drug side effects and treatment resistance has been documented in various case reports. Optimally, DNA typing could be performed prior to drug prescription in order to optimize therapy at the outset of psychotropic management. Those of skill in the art will be identify and associate these and other genes within each of the invention categories.

A preferred assessment table is provided below in Table 1.

TABLE 1

| Genes and phenotypes (markers) | Outcomes | Genotypes |
|---|---|---|
| CYP2D6 and drug metabolism | Poor metabolizer | |
| | Intermediate | |
| | metabolizer | |
| | Extensive metabolizer | |
| | Ultrarapid metabolizer | |
| CYP2C19 and drug metabolism | Poor metabolizer | |
| | Intermediate | |
| | metabolizer | |
| | Extensive metabolizer | |
| | Ultrarapid metabolizer | |
| CYP1A2 and drug metabolism (rs762551) | Fast metabolizer | A/A |
| | Slow metabolizer | A/C, C/C |
| UGT1A4 and drug metabolism (rs2011425) | Fast metabolizer | G/G, G/T |
| | Typical metabolizer | T/T |
| SLC6A4 and antidepressant treatment (5- | Decreased benefit | S/S, L(G)/L(G), S/L(G) |
| HTTLPR and rs25531) | Typical benefit | L(A)/S, L(A)/L(G) |
| | Increased benefit | L(A)/L(A) |
| HTR2A and citalopram response | Increased | A/A |
| (rs7997012) | Typical | A/G |
| | Decreased | G/G |
| HTR2A and adverse reactions to paroxetine | Increased risk with | G/G |
| and fluvoxamine (rs6311) | paroxetine | |
| | Typical risk | G/A |
| | Decreased risk with | A/A |
| | fluvoxamine | |
| HTR2C and atypical antipsychotic-induced | Typical risk | C/C, C |
| weight gain (rs3813929) | Decreased risk | T/C, T/T, T |
| DRD2 and risperidone response | Typical | Ins/Ins |
| (rs1799732) | Decreased | Del/Del, Del/Ins |
| HLA-B and anticonvulsant hypersensitivity | Increased risk | carrier of HLA-B*1502 |
| (rs3909184, rs2844682) | Typical risk | not carrier of HLA- |
| | | B*1502 |
| | Unknown | het at both tag SNPs |

Additional genes are described in Table 2 in Addendum A attached hereto.

### Risk

In parallel or in addition to the above, the present invention further comprises methods of determining a predisposition or susceptibility of a subject to a mood disorder, schizophrenia, or other mental or psychiatric disease or disorder, generally comprising detecting the presence of genetic variations to genes associated with a mental or psychiatric disease or disorder. These genes may be distinct or identical to the genes identified herein, e.g., a genetic variation to a mental disorder may be underlying cause of the mental or psychiatric disease or disorder.

GRK3

The GRK3 gene maps to human chromosome 22q11, and is also referred to as “beta adrenergic receptor kinase 2” (BARK2). This region has been implicated in bipolar disorder by the present inventors and others (See e.g., Lachman et al., Am. J Med. Genet. 74:121 [1996]; Kelsoe et al., Am. J Med. Genet. 81:461 [Abstract] [1998]; Edenberg et al., Am. J Med. Genet. 74:238 [1997]; and Detera-Wadleigh et al., Proc. Natl. Acad. Sci. USA 96:5604 [1999]). Indeed, 22q yielded the highest lod scores of any chromosomal region in the genome survey utilized during development of the present invention. Consistent with many findings in this field, this linkage peak was broad and spanned nearly 20 cM. One of the highest lod scores in this region was 2.2 at D22S419, which maps to within 40 kb of GRK3. This marker is also quite close to the markers identified in the two other independent positive linkage reports for 22q in bipolar disorder. A marker within the GRK3 gene, D22S315, has also been implicated in a study of eye tracking and evoked potential abnormalities in schizophrenia (See, Myles-Worsley et al., Am. J. Med. Genet. 88:544 [1999]).

The known physiological role of GRK3 in desensitization of receptors and its map location make it one of the more interesting candidates identified during the development of the present invention. In the continuing presence of high agonist concentrations, G protein-coupled receptor (GPCR) signaling is rapidly terminated by a process termed “homologous desensitization.” Homologous desensitization of many agonist-activated GPCRs begins when G protein receptor kinases (GRKs) phosphorylate serine and threonine residues on the receptor's cytoplasmic tail and/or third intracellular loop (Pitcher et al., Ann. Rev. Biochem. 67:653 [1998]). The consequent binding of β-arrestin to phosphorylated GPCRs decreases their affinity for cognate heterotrimeric G proteins, thereby uncoupling the receptor from the G-βγ subunit by steric hindrance. In addition, dopamine D1 receptors can be phosphorylated and desensitized via a GRK3 mechanism (Tiberi et al., J. Biol. Chem. 271:3771 [1996]). Also, GRK3 expression is particularly high in doparninergic pathways in the central nervous system (Arriza et al., J. Neurosci. 12:4045 [1992]). While an understanding of the mechanism(s) is not necessary in order to use the present invention, these data are consistent with results observed during the development of the present invention that indicate GRK3 exerts an important regulatory effect on brain dopamine receptors. Because dopamine receptors play an important role in the action of amphetamine on the brain, it is believed that amphetamine-induced up-regulation of GRK3 counter-regulates dopamine receptor signalling initiated by mesocorticolimbic dopamine release. Indeed, this gene undergoes a dramatic up-regulation in rat frontal cortex in response to amphetamine challenge. However, it is not intended that the present invention be limited to any particular mechanism(s).

These data suggest that an apparent major physiological role for GRK3 in neurons is to act as a brake to limit excessive neural activity by inactivating G protein-coupled receptors. It is contemplated that defects in GRK3 function are associated with the inability to desensitize, resulting in a heightened responsiveness to dopamine signals in the brain. It is contemplated that in at least some cases, such genetic variation influences individual variation in behavioral sensitization to stimulants in humans and other animals. It is further contemplated that the present invention will provide means to predict whether individuals with mania have either low levels of the normal protein or high levels of mutated hypoactive protein. Conversely, it is contemplated that individuals with depression have either high levels of the normal protein or normal levels of mutated hyperactive protein. Indeed this predictive model is supported by post-mortem studies in people who had depression that led to suicide and who had increased levels of GRK2/3 protein in their PFC (Garcaia-Sevilla et al., J. Neurochem. 72:282 [1999]).

In order to test this hypothesis, levels of GRK3 protein in lymphoblastoid cell lines of individuals with bipolar disorder from families with evidence of linkage to 22q11 were tested (See, Example 5). Consistent with this model, three out of six such subjects demonstrated reduced expression of GRK3. These data suggest that a defect in transcriptional regulation in GRK3 contributes to the susceptibility to bipolar disorder in a subset of individuals. Thus, functional defects in this gene appear to prevent the normal desensitization to dopamine or other neurotransmitters, resulting in predisposition to psychiatric disorder(s).

During the development of the present invention, it was also determined that the defect in GRK3 appears to be a variation in sequences that regulate transcription of the gene. The gene was screened and no evidence of coding sequence defects was found. However, six sequence variants that may affect promoter function were identified (See, Example 3 and FIGS. 1 and 2). Thus, it is contemplated that the present invention will find use in screening and identifying drugs that augment GRK3 expression and/or function.

### D Box Binding Protein (DBP)

D box binding protein (DBP) is a CLOCK-controlled transcriptional activator (Ripperger et al., Genes Dev. 14:679 [2000]), that shows a robust circadian rhythm. In mouse experiments (Yan et al., J. Neurosci. Res. 59:291 [2000]), its highest level of expression in the brain was found to be in the suprachaismatic nucleus (SCN), but it is also present in the cerebral cortex and caudate-putamen. In the SCN, DBP mRNA levels showed a peak at early daytime (ZT/CT4) and a trough at early nighttime in both light-dark and constant dark conditions. In the cerebral cortex and caudate-putamen, DBP mRNA was also expressed in a circadian manner, but the phase shift of DBP mRNA expression in these structures showed a 4-8 hour delay compared to the SCN. These data implicate DBP as an arm of the circadian clock. DBP knockout mice show reduced amplitude of the circadian modulation of sleep time, as well as a reduction in the consolidation of sleep episodes (Franken et al., J. Neurosci. 20:617 [2000]). Some clock genes have been shown to be essential for the development of behavioral sensitization to repeated stimulate exposure (Andretic et al., Science 285:1066 [1999]). Circadian rhythm abnormalities have also been implicated in mood disorders (See e.g., Kripke et al., Biol. Psychiatr. 13:335 [1978]; and Bunney and Bunney, Neuropsychopharmacol. 22:335 [2000]).

DBP maps to chromosome 19q13.3. Chromosome 19 has not been a strong linkage region for psychiatric disorders, although one study has implicated this region in a large Canadian kindred with bipolar disorder (Morissette et al., Am. J. Med. Genet. 88:567 [1999]). In this sample, D19S867, which is approximately 2 cM from DBP yielded a lod score of 2.6. Taken together, the connections between clock genes, stimulant sensitization and circadian rhythmicity suggest a potential role for DBP in mood disorders.

### Farnesyl-diphosphate Farnesyltransferase 1 (FDFT1)

FDFT1, also known as “human squalene synthase” (HSS), is involved in the first step of sterol biosynthesis uniquely committed to the synthesis of cholesterol (Schechter et al., Genomics 20:116 [1994]). As such, it has received attention as a target for the development of cholesterol-lowering drugs. Interestingly, primary prevention human trials have shown a correlation between lowering cholesterol and suicide, postulated to occur due to lowering the numbers of serotonin receptors in synapses (Engelberg, Lancet 339:727 [1992]). Studies in monkeys have also shown an association between cholesterol and central serotonergic activity (Kaplan et al., Ann. NY Acad. Sci. 836:57 [1997]). Mice homozygously disrupted for the squalene synthase gene exhibited embryonic lethality and defective neural tube closure, implicating de novo cholesterol synthesis in nervous system development (Tozawa et al., J. Biol. Chem. 274:30843 [1999]). Moreover, de novo cholesterol synthesis was shown to be important for neuronal survival., and apoE4, which is a major risk factor for Alzheimer's disease, has been implicated in inducing neuronal cell death through the suppression of de novo cholesterol synthesis (Michikawa and Yanagisawa, Mech. Ageing Dev. 107:223 [1999]). As such, it is contemplated that neuronal cholesterol synthesis, of which squalene synthase is a key regulator, is positively correlated with both elevated mood and neuronal survival. Nonetheless, an understanding of the mechanism(s) is not necessary in order to use the present invention, nor is it intended that the present invention be limited to any particular mechanism(s).

FDFT1 is located on 8p23.1-p22, near the telomere. Numerous studies have implicated 8p in both schizophrenia and bipolar disorder. However, most of these results are about 40-50 cM centromeric to FDFT1. Two studies have reported evidence for linkage to schizophrenia within 10 cM of FDFT1. Wetterberg et al. (Wetterberg et al., Am. J. Med. Genet. 81:470 [Abstract] [1998]), reported a lod score of 3.8 at D8S264, in a large Swedish isolate. The NIMH Schizophrenia Genetics Consortium also reported evidence implicating a broad area of 8p in African American pedigrees, including two putative peaks, with one at D8S264 (NPL Z score 2.3) (Kaufinann et al., Am. J. Med. Genet. 81:282 [1998]).

### Vertebrate LIN7 Homolog 1 (MALS-1 or VELI1)

MALS-1 is a PDZ domain-containing cytoplasmic protein that is enriched in brain synapses where it associates in complexes with PSD-95 and NMDA type glutamate receptors (Jo et al., J. Neurosci. 19:4189 [1999]). It has been implicated in regulation of neurotransmitter receptor recruitment to the post-synaptic density, as well as being part of a complex with CASK and Mint 1 that couples synaptic vesicle exocytosis to cell adhesion (Butz et al., Cell 94:773 [1998]).

MALS-1 maps to 12q21.3, in a region implicated in several studies of bipolar disorder. This region was first reported in bipolar disorder through observation of a Welsh family in which bipolar disorder and Darier's disease co-segregated (Dawson et al., Am. J. Med. Genet. 60:94 [1995]). Though the Darier's region is somewhat distal to MALS-1, Morisette et al. reported evidence of linkage of bipolar disorder to markers on 12q, with a maximum at D12582 (Zall 4.0, lod score 2.2), which is approximately 2 cM from MALS-1 (Morisette et al., supra).

### E. Sulfotransferase 1 A1 (SULT1A1)

SULT1A1 is a sulfotransferase that inactivates dopamine and other phenol-containing compounds by sulfation. It is contemplated as playing a role in limiting the neuronal stimulatory and psychosis promoting effects of dopamine. Though it is not a primary regulator of synaptic dopamine concentration, a defect in this gene could lead to impaired clearing of dopamine from the extracellular space with a resulting amphetamine-like effect. SULT1A1 has not yet been precisely mapped, but cytogenetic data locate it to chromosome 16p12.1-p11.2, near a genomic locus implicated in bipolar disorder (D165510, lod score 2.5) (Ewald et al., Psychiatr. Genet. 5:71 [1995]), and alcohol dependence (D165675, lod score 4.0)(Foroud et al., Alcohol Clin. Exp. Res. 22:2035 [1998]).

### Insulin-Like Growth Factor 1 (IGF1)

IGF1 stimulates increased expression of tyrosine hydroxylase, the rate limiting enzyme in the biosynthesis of dopamine (Hwang and Choi, J. Neurochem. 65:1988 [1995]). It has also been shown to have trophic effects on doparnine brain neurons and to protect doparnine neurons from apoptotic death (Knusel et al., Adv. Exp. Med. Biol. 293:351 [1991]). IGF1 also induces phosphatidylinositol 3-kinase survival pathways through activation of AKT1 and AKT2; it is inhibited by TNF in its neuroprotective role. IGF1 gene disruption in mice results in reduced brain size, CNS hypomyelination, and loss of hippocampal granule and striatal parvalbumin-containing neurons (Beck et al., Neuron 14:717 [1995]). Defects of IGF1 in humans produce growth retardation with deafness and mental retardation. IGF1 is located on chromosome 12q22-q24.1. It is at a map position of 109 cM, 13 cM telomeric to MALS-1, and is in the same 40 cM region described above. This region is implicated in bipolar disorder and extends from D12S82 at 96 cM (NPL Zall 4.0) (Morisette et al., supra) to PLA2 at 136 cM (lod score 2.49) (Dawson et al., supra).

### Additional Genes

Two additional genes met the criteria of reproducibility and mapping to a linkage region, but their functions identified to date make them less likely to be disease gene candidates. RNA polymerase II polypeptide (POLR2F) maps to 22q13.1, approximately 10 cM distal to D22S278, which has been implicated in several studies of both bipolar disorder and schizophrenia, as described above. POLR2F is responsible for mRNA production and may control cell size (Schmidt and Schibler, J. Cell Biol. 128:467 [1995]), and overall body morphological features (Bina et al., Prog. Nucl. Acid Res. Mol. Biol. 64:171 [2000]). It is more active in metabolically active cells (Schmidt and Schibler, supra). FCGRT is a receptor for the Fc component of IgG. It structurally resembles the major histocompatibility class I molecule (Kandil et al., Cytogenet. Cell Genet. 73:97 [1996]). FCGRT maps to 19q13.3, near DBP and a marker implicated in bipolar disorder, as discussed above. It is contemplated that activation of these genes is a secondary effect of amphetamine and their mapping near linkage regions is coincidental.

Several other genes did not meet the stringent criteria used in the development of the present invention. For example, fibroblast growth factor receptor 1 (FGFR1) had an average fold change of 4.1, though the increase was only 1.8 fold in one of the two experiments. Increased expression of astrocytic basic FGF in response to amphetamine was previously demonstrated (Flores et al., J. Neurosci. 18:9547 [1998]). Furthermore, FGF-2, a ligand for FGFR1 has been shown to regulate expression of tyrosine hydroxylase, a critical enzyme in dopamine biosynthesis (Rabinovsky et al., J. Neurochem. 64:2404 [1995]). FGFR1 maps to chromosome 8p11.2-p11.1, approximately 10 cM centromeric to a genomic locus near D8D1771 (8p22-24), which demonstrated evidence of linkage to schizophrenia in several studies (See e.g. Blouin et al., Nat. Genet. 20:70 [1998]; Kendler et al., Am. J Psychiatr. 153:1534 [1996]; and Levinson et al., Am. J. Psychiatr. 155:741 [1998]). Heat shock 27 kD protein 1 (H5P27, HSPB1) has been implicated in stress resistance responses in a variety of tissues. It is hypothesized that it plays a role in promoting neuronal survival (See e.g. Lewis et al., J. Neurosci. 19:8945 [1999]), and may be induced in the brain by kainic acid-induced seizure (Kato et al., J. Neurochem. 73:229 [1999]). HSPB1 maps to 7q22.1, approximately 20 cM from a region implicated in bipolar disorder in two independent samples (Detera-Wadleigh et al., Am. J. Med. Genet. 74:254 [1997]; and Detera-Wadleigh et al., Proc. Natl. Acad. Sci. USA 96:5604 [1999]).

SNPs at four loci surpassed the cutoff for genome-wide significance (p<5×10-8) in the primary analysis: regions on chromosomes 3p21 and 10q24, and SNPs within two L-type voltage-gated calcium channel subunits, CACNA1C and CACNB2. Model selection analysis supported effects of these loci for several disorders. Loci previously associated with bipolar disorder or schizophrenia had variable diagnostic specificity. Polygenic risk scores showed cross-disorder associations, notably between adult-onset disorders. Pathway analysis supported a role for calcium channel signaling genes for five disorders, autism spectrum disorder, attention deficit-hyperactivity disorder, bipolar disorder, major depressive disorder, and schizophrenia. Smoller J W, et al “Identification of risk loci with shared effects on five major psychiatric disorders: a genome-wide analysis” Lancet. Lancet. 2013 Apr. 20; 381(9875):1371-9 (Erratum in 2013 Apr. 20; 381(9875):1360).

Additional markers are found in the attachments hereto.

### Diagnostic Methods

The invention further features diagnostic medicines, which are based, at least in part, on determination of the identity of the polymorphic region or expression level (or both in combination) of the genetic markers above.

For example, information obtained using the diagnostic assays described herein is useful for determining if a subject will respond to treatment for a given indication. Based on the prognostic information, a doctor can recommend a therapeutic protocol, useful for prescribing different treatment protocols for a given individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. Expression patterns of individual patients can then be compared to the expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Genotyping of an individual can be initiated before or after the individual begins to receive treatment.

Side effects of a particular treatment are those related to treatment based on a positive correlation between frequency or intensity of occurrence and drug treatment. Such information is usually collected in the course of studies on efficacy of a drug treatment and many methods are available to obtain such data. Resulting information is widely distributed among the medical profession and patients receiving treatment.

A treatment result is defined here from the point of view of the treating doctor, who judges the efficacy of a treatment as a group result. Within the group, individual patients can recover completely and some may even worsen, due to statistical variations in the course of the disease and the patient population. Some patients may discontinue treatment due to side effects, in which case no improvement in their condition due to psychiatric medication treatment can occur. An improved treatment result is an overall improvement assessed over the whole group. Improvement can be solely due to an overall reduction in frequency or intensity of side effects. It is also possible that doses can be increased or the dosing regime can be stepped up faster thanks to less troublesome side effects in the group and consequently an earlier onset of recovery or better remission of the disease.

A disorder, which is responsive to treatment with a particular drug or treatment, is defined to be a disorder, which is, according to recommendations in professional literature and drug formularies, known to respond with at least partial remission of the symptoms to a treatment with such drug or treatment. In most countries such recommendations are subject to governmental regulations, allowing and restricting the mention of medical indications in package inserts. Other sources are drug formularies of health management organizations. Before approval by governmental agencies certain recommendations can also be recognized by publications of confirmed treatment results in peer reviewed medical journals. Such collective body of information defines what is understood here to be a disorder that is responsive to treatment with an particular medication. Being responsive to particular treatment does not exclude that the disorder in an individual patient can resist treatment with such treatment, as long as a substantial portion of persons having the disorder respond with improvement to the treatment.

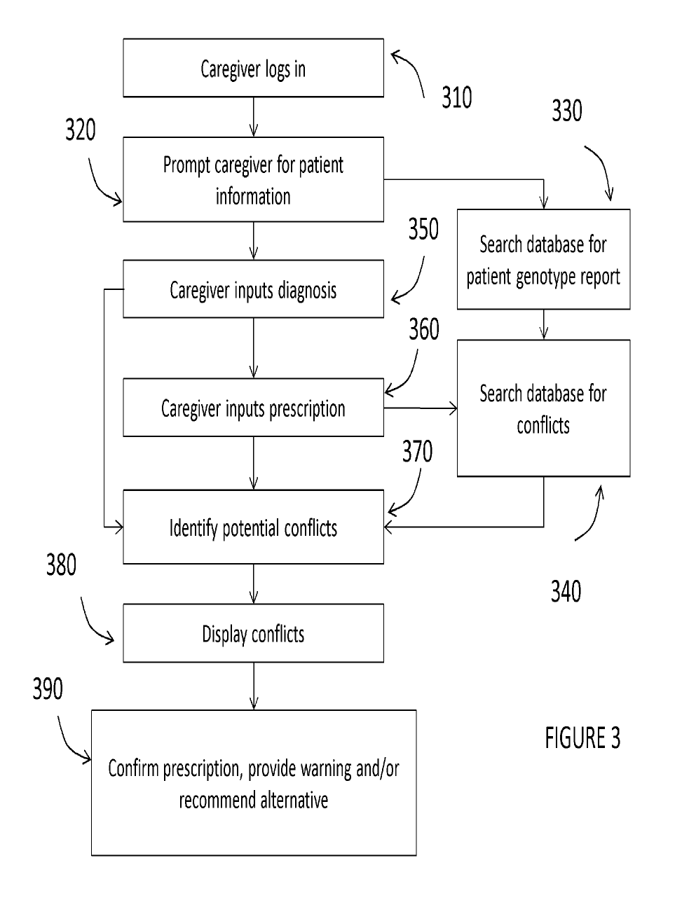

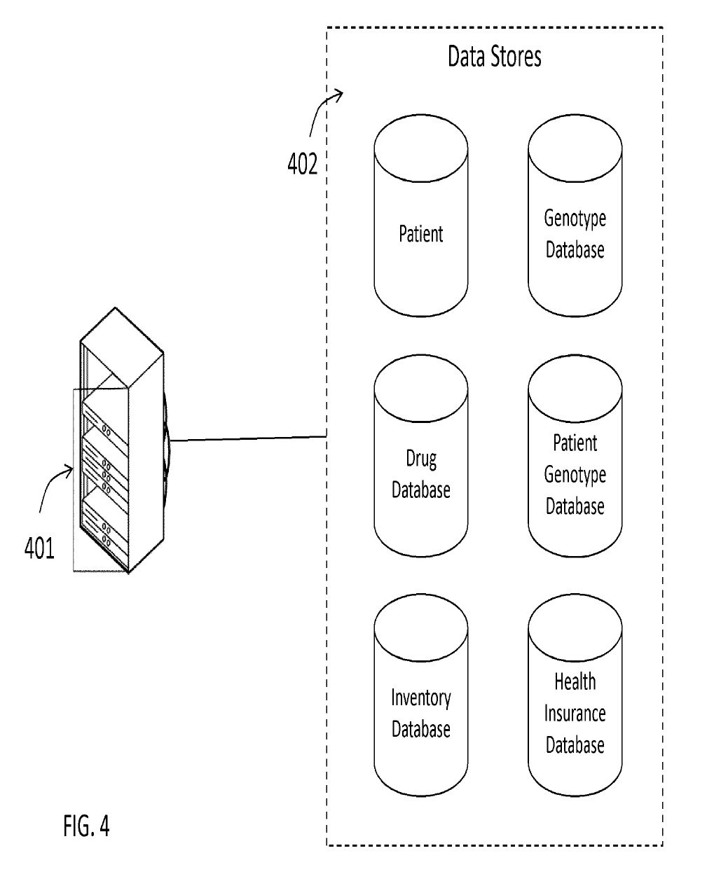

In a particular embodiment of the present invention, there are provided a method and system for healthcare providers (e.g., caregiver, physicians, doctors, nurses, pharmacists, insurance companies, therapist, medical specialists such as psychiatrists, etc.), or other to access information about the genetic profile of an individual to recommend or warn about particular treatments. FIG. 3 displays an interactive process of a healthcare provider, or individual with the invention system for recommending particular medications. A caregiver can access information 310 of their patient by accessing the system and interacting with the patient genetic records. As the system is targeted to providing personal information, the system will require the identity of the individual 320 to analyze or report upon. This information may be accessed 330 through information stored onsite or offsite in, for example, a patient data warehouse or with a laboratory or company providing such services. Either the system and/or the caregiver can provide additional information such as the diagnosis 350 (e.g., the genotyping may consist of analyzing an individual to detect genetic anomalies associated with the disorder or disease). Further, the caregiver can input any recommended prescriptions 360 that can be analyzed 340 against the individual's genetic profile to determine the efficacy and/or risk of such a treatment protocol. Any potential conflicts and problems can be flagged 370 and displayed 380 for the caregiver to review. Alternatively, the system can recommend or warn against particular medications and treatments, or classes of medications or treatments upon analysis of the individual's genetic profile. Once any warnings or recommendations are made, the system can further confirm the determination of the caregiver, provide additional warnings or alternative medications or treatments 390. The system 401 can be tied, as shown in FIG. 4, into one or more additional databases 402 to further analyze inventory, price, insurance restrictions and the like.

Various embodiments of the invention provide for methods for identifying a genetic variation (e.g, allelic patterns, polymorphism patterns such as SNPs, or haplotype patterns etc.), comprising collecting biological samples from one or more subjects and exposing the samples to detection assays under conditions such that the presence or absence of at least one genetic variation is revealed. To begin, polynucleotide samples derived from (e.g., obtained from) an individual may be employed. Any biological sample that comprises a polynucleotide from the individual is suitable for use in the methods of the invention. The biological sample may be processed so as to isolate the polynucleotide. Alternatively, whole cells or other biological samples may be used without isolation of the polynucleotides contained therein.

Detection of a genetic variation in a polynucleotide sample derived from an individual can be accomplished by any means known in the art, including, but not limited to, amplification of a sequence with specific primers; determination of the nucleotide sequence of the polynucleotide sample; hybridization analysis; single strand conformational polymorphism analysis; denaturing gradient gel electrophoresis; mismatch cleavage detection; and the like. Detection of a genetic variation can also be accomplished by detecting an alteration in the level of a mRNA transcript of the gene; aberrant modification of the corresponding gene, e.g., an aberrant methylation pattern; the presence of a non-wild-type splicing pattern of the corresponding mRNA; an alteration in the level of the corresponding polypeptide; determining the electrophoretic mobility of the allele or fragments thereof (e.g., fragments generated by endonuclease digestion), and/or an alteration in corresponding polypeptide activity.

In some embodiments, a subject can be genotyped for an allele, more preferably a polymorphism by collecting and assaying a biological sample of the patient to determine the nucleotide sequence of the gene at that polymorphism, the amino acid sequence encoded by the gene at that polymorphism, or the concentration of the expressed product, e.g., by using one or more genotyping reagents, such as but not limited to nucleic acid reagents, including primers, etc., which may or may not be labeled, amplification enzymes, buffers, etc. In certain embodiments, the target polymorphism will be detected at the protein level, e.g., by assaying for a polymorphic protein. In yet other embodiments, the target polymorphism will be detected at the nucleic acid level, e.g., by assaying for the presence of nucleic acid polymorphism, e.g., a single nucleotide polymorphism (SNP) that cause expression of the polymorphic protein. Any convenient protocol for assaying a sample for the above one or more target polymorphisms may be employed in the subject methods.

In general, nucleic acid is extracted from the biological sample using conventional techniques. The nucleic acid to be extracted from the biological sample may be DNA, or RNA, typically total RNA. Typically RNA is extracted if the genetic variation to be studied is situated in the coding sequence of a gene. Where RNA is extracted from the biological sample, the methods further comprise a step of obtaining cDNA from the RNA. This may be carried out using conventional methods, such as reverse transcription using suitable primers. Subsequent procedures are then carried out on the extracted DNA or the cDNA obtained from extracted RNA. The term DNA, as used herein, may include both DNA and cDNA.

In general the genetic variations to be tested are known and characterised, e.g. in terms of sequence. Therefore nucleic acid regions comprising the genetic variations may be obtained using methods known in the art.

In one aspect, DNA regions which contain the genetic variations to be identified (target DNA regions) are subjected to an amplification reaction in order to obtain amplification products that contain the genetic variations to be identified. Any suitable technique or method may be used for amplification. In general, the technique allows the (simultaneous) amplification of all the DNA sequences containing the genetic variations to be identified. In other words, where multiple genetic variations are to be analysed, it is preferable to simultaneously amplify all of the corresponding target DNA regions (comprising the variations). Carrying out the amplification in a single step (or as few steps as possible) simplifies the method.

Analyzing a polynucleotide sample can be conducted in a number of ways. Preferably, the allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). A test nucleic acid sample can be amplified with primers that amplify a region known to comprise the target polymorphism(s), for example, from within the metabolic gene loci, either flanking the marker of interest (as required for PCR amplification) or directly overlapping the marker (as in allele specific oligonucleotide (ASO) hybridization). In a particularly preferred embodiment, the sample is hybridized with a set of primers, which hybridize 5′ and 3′ in a sense or antisense sequence to the vascular disease associated allele, and is subjected to a PCR amplification. Genomic DNA or mRNA can be used directly or indirectly, for example, to convert into cDNA. Alternatively, the region of interest can be cloned into a suitable vector and grown in sufficient quantity for analysis.

The nucleic acid may be amplified by conventional techniques, such as a polymerase chain reaction (PCR), to provide sufficient amounts for analysis. The use of the polymerase chain reaction is described in a variety of publications, including, e.g., “PCR Protocols (Methods in Molecular Biology)” (2010) Daniel J. Park, eds, (Humana Press, 3^{rd} ed. (2011); and Saunders N A & Lee, M A. Eds “Real-Time PCR: Advanced Technologies and Applications (Caister Academic Press (2013). Other methods for amplification of nucleic acids is ligase chain reaction (“LCR”), disclosed in European Application No. 320 308, isothermal amplification method, such as described in Walker et al., (Proc. Nat'l Acad. Sci. USA 89:392-396, 1992) or Strand Displacement Amplification or Repair Chain Reaction (RCR), transcription-based amplification systems (TAS), including nucleic acid sequence based amplification (NASBA) and 3SR. Kwoh et al., Proc. Nat'l Acad. Sci. USA 86:1173 (1989); Gingeras et al., PCT Application WO 88/10315, cyclic and non-cyclic synthesis of single-stranded RNA (“ssRNA”), ssDNA, and double-stranded DNA (dsDNA) (Davey et al., European Application No. 329 822 and Miller et al., PCT Application WO 89/06700, respectively) and di-nucleotide amplification (Wu et. al., Genomics 4:560 1989). Miller et al., PCT Application WO 89/06700 Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197, PCT Application No. PCT/US87/00880), or any other nucleic acid amplification method (e.g., GB Application No. 2 202 328, and in PCT Application No. PCT/US89/01025), followed by the detection of the amplified molecules using techniques known to those of skill in the art. These detection schemes are useful for the detection of nucleic acid molecules if such molecules are present in very low numbers.

Once the region of interest has been amplified, the genetic variant of interest can be detected in the PCR product by nucleotide sequencing, by SSCP analysis, or any other method known in the art. In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad Sci, USA 74:560 or Sanger et al. (1977) Proc. Nat. Acad. Sci, 74:5463. It is also contemplated that any of a variety of automated sequencing procedures can be utilized when performing the subject assays (Biotechniques (1995) 19:448), including by mass spectrometry (see, for example, U.S. Pat. No. 5,547,835 and International Patent Application Publication Number WO94/16101, entitled DNA Sequencing by Mass Spectrometry by H. Koster; U.S. Pat. No. 5,547,835 and international patent application Publication No. WO 94/21822 entitled “DNA Sequencing by Mass Spectrometry Via Exonuclease Degradation” by H. Koster; U.S. Pat. No. 5,605,798 and International Patent Application No. PCT/US96/03651 entitled DNA Diagnostics Based on Mass Spectrometry by H. Koster; Cohen et al. (1996) Adv. Chromat. 36:127-162; and Griffin et al. (1993) Appl Biochem Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

The high demand for low-cost sequencing has driven the development of high-throughput sequencing (or next-generation sequencing) technologies that parallelize the sequencing process, producing thousands or millions of sequences concurrently. High-throughput sequencing including ultra-high-throughput sequencing technologies are intended to lower the cost of DNA sequencing beyond what is possible with standard dye-terminator methods. These methods include pyrosequencing, reversible dye-terminator (Bentley, D. R.; Balasubramanian, S.; Swerdlow, H. P.; Smith, G. P.; Milton, J.; Brown, C. G.; Hall, K. P.; Evers, D. J. et al. (2008). “Accurate whole human genome sequencing using reversible terminator chemistry”. Nature 456 (7218): 53-59), SOLiD sequencing using sequencing by ligation Valouev A, Ichikawa J, Tonthat T et al. (July 2008). “A high-resolution, nucleosome position map of C. elegans reveals a lack of universal sequence-dictated positioning”. Genome Res. 18 (7): 1051-6), ion semiconductor sequencing (Rusk N (2011). “Torrents of sequence”. Nat Meth 8 (1): 44-44), Heliscope (single molecule sequencing (Helicos Biosciences, Thompson, J F; Steinmann, K E (2010 October). “Single molecule sequencing with a HeliScope genetic analysis system.”. Current protocols in molecular biology/edited by Frederick M. Ausubel . . . [et al.] Chapter 7: Unit7.10), single molecule real-time (SMRT) sequencing (Pacific Biosciences; M. J. Levene, J. Korlach, S. W. Turner, M. Foquet, H. G. Craighead, W. W. Webb, Zero-Mode Waveguides for Single-Molecule Analysis at high concentrations. Science. 299 (2003) 682-686), nanopore DNA sequencing (M. J. Levene, J. Korlach, S. W. Turner, M. Foquet, H. G. Craighead, W. W. Webb, Zero-Mode Waveguides for Single-Molecule Analysis at high concentrations. Science. 299 (2003) 682-686), hybridization sequencing (Hanna G J, Johnson V A, Kuritzkes D R et al. (1 Jul. 2000). “Comparison of Sequencing by Hybridization and Cycle Sequencing for Genotyping of Human Immunodeficiency Virus Type 1 Reverse Transcriptase”. J. Clin. Microbiol. 38 (7): 2715-21), mass spectrometry sequencing (J. R. Edwards, H. Ruparel, and J. Ju (2005). “Mass-spectrometry DNA sequencing”. Mutation Research 573 (1-2): 3-12), Sanger microfluidic sequencing (Ying-Ja Chen, Eric E. Roller and Xiaohua Huang (2010). “DNA sequencing by denaturation: experimental proof of concept with an integrated fluidic device”. Lab on Chip 10 (10): 1153-1159), microscopy-based techniques such as transmission electron microscopy DNA sequencing (Ying-Ja Chen, Eric E. Roller and Xiaohua Huang (2010). “DNA sequencing by denaturation: experimental proof of concept with an integrated fluidic device”. Lab on Chip 10 (10): 1153-1159), RNA polymerase (RNAP) (Pareek, C S; Smoczynski, R; Tretyn, A (2011 November). “Sequencing technologies and genome sequencing.”. Journal of applied genetics 52 (4): 413-35), in vitro virus high-throughput sequencing (Fujimori, S; Hirai, N; Ohashi, H; Masuoka, K; Nishikimi, A; Fukui, Y; Washio, T; Oshikubo, T; Yamashita, T; Miyamoto-Sato, E (2012). “Next-generation sequencing coupled with a cell-free display technology for high-throughput production of reliable interactome data.”. Scientific reports 2: 691), and the like.

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

In preferred embodiments of the present invention, variant sequences are detected using a hybridization assay. In a hybridization assay, the presence of absence of a given SNP or mutation is determined based on the ability of the DNA from the sample to hybridize to a complementary DNA molecule (e.g., a oligonucleotide probe). Parameters such as hybridization conditions, polymorphic primer length, and position of the polymorphism within the polymorphic primer may be chosen such that hybridization will not occur unless a polymorphism present in the primer(s) is also present in the sample nucleic acid. Those of ordinary skill in the art are well aware of how to select and vary such parameters. See, e.g., Saiki et al. (1986) Nature 324:163; and Saiki et al (1989) Proc. Natl. Acad. Sci. USA 86:6230.

Yet other sequencing methods are disclosed, e.g., in U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In Vitro DNA Sequencing.”

In some cases, the presence of the specific allele in DNA from a subject can be shown by restriction enzyme analysis. For example, the specific nucleotide polymorphism can result in a nucleotide sequence comprising a restriction site that is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

Over or under expression of a gene, in some cases, is correlated with a genomic polymorphism. The polymorphism can be present in an open reading frame (coded) region of the gene, in a “silent” region of the gene, in the promoter region, or in the 3′untranslated region of the transcript. Methods for determining polymorphisms are well known in the art and include, but are not limited to, the methods discussed below.

Detection of point mutations or additional base pair repeats (as required for the polymorphism) can be accomplished by molecular cloning of the specified allele and subsequent sequencing of that allele using techniques known in the art. Alternatively, the gene sequences can be amplified directly from a genomic DNA preparation from the sample using PCR, and the sequence composition is determined from the amplified product. As described more fully below, numerous methods are available for analyzing a subject's DNA for mutations at a given genetic locus such as the gene of interest.

A detection method is allele specific hybridization using probes overlapping the polymorphic site and having about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244.

Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

In other embodiments, alterations in electrophoretic mobility are used to identify the particular allelic variant. For example, single strand conformation polymorphism (SSCP) may be used to detect differences in electrophoretic mobility between mutant and wild type nucleic acids (Orita et al. (1989) Proc Natl. Acad. Sci. USA 86:2766; Cotton (1993) Mutat. Res. 285:125-144 and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In performing SSCP analysis, the PCR product may be digested with a restriction endonuclease that recognizes a sequence within the PCR product generated by using as a template a reference sequence, but does not recognize a corresponding PCR product generated by using as a template a variant sequence by virtue of the fact that the variant sequence no longer contains a recognition site for the restriction endonuclease.

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

In another embodiment, identification of the allelic variant is carried out using an oligonucleotide ligation assay (OLA), as described, e.g., in U.S. Pat. No. 4,998,617 and in Landegren, U. et al. Science 241:1077-1080 (1988). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In one embodiment, the single base polymorphism can be detected by using a specialized exonuclease-resistant nucleotide, as disclosed, e.g., in Mundy (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site. Cohen et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet et al. supra, is preferably a heterogeneous phase assay, in which the primer or the target molecule is immobilized to a solid phase.

Recently, several primer-guided nucleotide incorporation procedures for assaying polymorphic sites in DNA have been described (Komher et al. (1989) Nucl. Acids. Res. 17:7779-7784; Sokolov (1990) Nucl. Acids Res. 18:3671; Syvanen et al. (1990) Genomics 8:684-692; Kuppuswamy et al. (1991) Proc. Natl. Acad. Sci. (U.S.A.) 88:1143-1147; Prezant et al. (1992) Hum. Mutat. 1:159-164; Ugozzoli et al. (1992) GATA 9:107-112; Nyren et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen et al. (1993) Amer. J. Hum. Genet. 52:46-59).

In one aspect the invention provided for a panel of genetic markers selected from, but not limited to the genetic polymorphisms above. The panel comprises probes or primers that can be used to amplify and/or for determining the molecular structure of the polymorphisms identified above. The probes or primers can be attached or supported by a solid phase support such as, but not limited to a gene chip or microarray. The probes or primers can be detectably labeled. This aspect of the invention is a means to identify the genotype of a patient sample for the genes of interest identified above. In one aspect, the methods of the invention provided for a means of using the panel to identify or screen patient samples for the presence of the genetic marker identified herein. In one aspect, the various types of panels provided by the invention include, but are not limited to, those described herein. In one aspect, the panel contains the above identified probes or primers as wells as other, probes or primers. In an alternative aspect, the panel includes one or more of the above noted probes or primers and others. In a further aspect, the panel consist only of the above-noted probes or primers.

In one embodiment of the invention, probes are labeled with two fluorescent dye molecules to form so-called “molecular beacons” (Tyagi and Kramer (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis (1998) Science 279:1228-9) as has the use of multiple beacons simultaneously (Marras (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a polymorphism. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule—quencher molecule pair that specifically anneals to a region of a target polynucleotide containing the polymorphism.

Probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are known in the art. Examples of such include, but are not limited to LabCard (ACLARA Bio Sciences Inc.); GeneChip (Affymetrix, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc., San Diego WO 99/67641 and WO 00/39587); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); surface tension array (ProtoGene, Palo Alto, Calif. U.S. Pat. Nos. 6,001,311; 5,985,551; and 5,474,796), BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in US Patent Publ. Nos.: 2007-0111322, 2007-0099198, 2007-0084997, 2007-0059769 and 2007-0059765 and U.S. Pat. Nos. 7,138,506, 7,070,740, and 6,989,267.

In one aspect, “gene chips” or “microarrays” containing probes or primers for genes of the invention alone or in combination are prepared. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genotypes of the patient is then determined with the aid of the aforementioned apparatus and methods.

An allele may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications. Methods for measuring gene expression are also well known in the art and include, but are not limited to, immunological assays, nuclease protection assays, northern blots, in situ hybridization, reverse transcriptase Polymerase Chain Reaction (RT-PCR), Real-Time Polymerase Chain Reaction, expressed sequence tag (EST) sequencing, cDNA microarray hybridization or gene chip analysis, statistical analysis of microarrays (SAM), subtractive cloning, Serial Analysis of Gene Expression (SAGE), Massively Parallel Signature Sequencing (MPSS), and Sequencing-By-Synthesis (SBS). See for example, Carulli et al., (1998) J. Cell. Biochem. 72 (S30-31): 286-296; Galante et al., (2007) Bioinformatics, Advance Access (Feb. 3, 2007).

SAGE, MPSS, and SBS are non-array based assays that determine the expression level of genes by measuring the frequency of sequence tags derived from polyadenylated transcripts. SAGE allows for the analysis of overall gene expression patterns with digital analysis. SAGE does not require a preexisting clone and can used to identify and quantitate new genes as well as known genes. Velculescu et al., (1995) Science 270(5235):484-487; Velculescu (1997) Cell 88(2):243-251.

MPSS technology allows for analyses of the expression level of virtually all genes in a sample by counting the number of individual mRNA molecules produced from each gene. As with SAGE, MPSS does not require that genes be identified and characterized prior to conducting an experiment. MPSS has a sensitivity that allows for detection of a few molecules of mRNA per cell. Brenner et al. (2000) Nat. Biotechnol. 18:630-634; Reinartz et al., (2002) Brief Funct. Genomic Proteomic 1: 95-104.

SBS allows analysis of gene expression by determining the differential expression of gene products present in sample by detection of nucleotide incorporation during a primer-directed polymerase extension reaction.

SAGE, MPSS, and SBS allow for generation of datasets in a digital format that simplifies management and analysis of the data. The data generated from these analyses can be analyzed using publicly available databases such as Sage Genie (Boon et al., (2002) PNAS 99:11287-92), SAGEmap (Lash et al., (2000) Genome Res 10:1051-1060), and Automatic Correspondence of Tags and Genes (ACTG) (Galante (2007), supra). The data can also be analyzed using databases constructed using in house computers (Blackshaw et al. (2004) PLoS Biol, 2:E247; Silva et al. (2004) Nucleic Acids Res 32:6104-6110)).

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject has or may have a greater or lower response to a particular treatment(s).

Diagnostic procedures can also be performed in situ directly upon samples from, such that no nucleic acid purification is necessary. Nucleic acid reagents can be used as probes and/or primers for such in situ procedures (see, for example, Nuovo (1992) “PCR 1N SITU HYBRIDIZATION: PROTOCOLS AND APPLICATIONS”, Raven Press, NY).

In addition to methods that focus primarily on the detection of one nucleic acid sequence, profiles can also be assessed in such detection schemes. Fingerprint profiles can be generated, for example, by utilizing a differential display procedure, Northern analysis and/or RT-PCR.

### Nucleic Acids

In one aspect, the nucleic acid sequences of the gene's allelic variants, or portions thereof, can be the basis for probes or primers, e.g., in methods and compositions for determining and identifying the allele present at the gene of interest's locus, more particularly to identity the allelic variant of a polymorphic region(s). Thus, they can be used in the methods of the invention to determine which therapy is most likely to affect or not affect an individual's disease or disorder, such as to diagnose and prognoses disease progression as well as select the most effective treatment among treatment options. Probes can be used to directly determine the genotype of the sample or can be used simultaneously with or subsequent to amplification.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers and probes for use in the methods of the invention are nucleic acids that hybridize to a nucleic acid sequence which is adjacent to the region of interest or which covers the region of interest and is extended. A primer or probe can be used alone in a detection method, or a can be used together with at least one other primer or probe in a detection method. Primers can also be used to amplify at least a portion of a nucleic acid. Probes for use in the methods of the invention are nucleic acids which hybridize to the region of interest and which are generally are not further extended. Probes may be further labeled, for example by nick translation, Klenow fill-in reaction, PCR or other methods known in the art, including those described herein). For example, a probe is a nucleic acid which hybridizes to the polymorphic region of the gene of interest, and which by hybridization or absence of hybridization to the DNA of a subject will be indicative of the identity of the allelic variant of the polymorphic region of the gene of interest. Probes and primers of the present invention, their preparation and/or labeling are described in Green and Sambrook (2012). Primers and Probes useful in the methods described herein are found in Table 1.

In one embodiment, primers and probes comprise a nucleotide sequence which comprises a region having a nucleotide sequence which hybridizes under stringent conditions to about 5 through about 100 consecutive nucleotides, more particularly about: 6, 8, 10, 12, 15, 20, 25, 30, 35, 40, 45, 50, 60, or 75 consecutive nucleotides of the gene of interest. Length of the primer or probe used will depend, in part, on the nature of the assay used and the hybridization conditions employed.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids that are capable of selectively hybridizing to an allelic variant of a polymorphic region of the gene of interest. Thus, such primers can be specific for the gene of interest sequence, so long as they have a nucleotide sequence that is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules that are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al., (1989) Proc. Natl. Acad. Sci. U.S.A. 86:6553-6556; Lemaitre et al., (1987) Proc. Natl. Acad. Sci. 84:648-652; and PCT Publication No. WO 88/09810, published Dec. 15, 1988), hybridization-triggered cleavage agents, (see, e.g., Krol et al., (1988) BioTechniques 6:958-976) or intercalating agents (see, e.g., Zon (1988) Pharm. Res. 5:539-549. To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook and Russel (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Kits

As set forth herein, the invention provides diagnostic methods for determining the type of allelic variant of a polymorphic region present in the gene of interest or the expression level of a gene of interest. In some embodiments, the methods use probes or primers comprising nucleotide sequences which are complementary to the polymorphic region of the gene of interest. Accordingly, the invention provides kits for performing these methods as well as instructions for carrying out the methods of this invention such as collecting tissue and/or performing the screen, and/or analyzing the results, and/or administration of an effective amount of the therapies described above.

In an embodiment, the invention provides a kit for determining whether a subject responds to treatment or alternatively one of various treatment options. The kits contain one of more of the compositions described above and instructions for use. As an example only, the invention also provides kits for determining response to treatment containing a first and a second oligonucleotide specific for the polymorphic region of the gene. Oligonucleotides “specific for” a genetic locus bind either to the polymorphic region of the locus or bind adjacent to the polymorphic region of the locus. For oligonucleotides that are to be used as primers for amplification, primers are adjacent if they are sufficiently close to be used to produce a polynucleotide comprising the polymorphic region. In one embodiment, oligonucleotides are adjacent if they bind within about 1-2 kb, and preferably less than 1 kb from the polymorphism. Specific oligonucleotides are capable of hybridizing to a sequence, and under suitable conditions will not bind to a sequence efficiently differing by a single nucleotide.

The kit can comprise at least one probe or primer which is capable of specifically hybridizing to the polymorphic region of the gene of interest and instructions for use. The kits preferably comprise at least one of the above described nucleic acids. Preferred kits for amplifying at least a portion of the gene of interest comprise two primers and two probes, at least one of probe is capable of binding to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard (1986) AN INTRODUCTION TO RADIOIMMUNOASSAY AND RELATED TECHNIQUES Elsevier Science Publishers, Amsterdam, The Netherlands; Bullock et al. TECHNIQUES IN IMMUNOCYTOCHEMISTRY Academic Press, Orlando, Fla. Vol. 1 (1982), Vol. 2 (1983), Vol. 3 (1985); Tijssen, PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands (1985).

The test samples used in the diagnostic kits include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region or the expression levels of the gene of interest.

As amenable, these suggested kit components may be packaged in a manner customary for use by those of skill in the art. For example, these suggested kit components may be provided in solution or as a liquid dispersion or the like.

Other Uses for the Nucleic Acids of the Invention

The identification of the allele of the gene of interest can also be useful for identifying an individual among other individuals from the same species. For example, DNA sequences can be used as a fingerprint for detection of different individuals within the same species. Thompson and Thompson, Eds., (1991) GENETICS IN MEDICINE, W B Saunders Co., Philadelphia, Pa. This is useful, e.g., in forensic studies.

The invention now being generally described, it will be more readily understood by reference to the following examples which are included merely for purposes of illustration of certain aspects and embodiments of the present invention, and are not intended to limit the invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, histology and immunology. Such procedures are described, for example, in the following texts that are incorporated by reference:

(i) Green M R, Sambrook J, Molecular Cloning: A Laboratory Manual, Cold Spring Harbor Laboratories Press, New York, Fourth Edition (2012), whole of Vols I, II, and III;

(ii) DNA Cloning: A Practical Approach, Vols. I-IV (D. M. Glover, ed., 1995), Oxford University Press, whole of text;

(iii) Oligonucleotide Synthesis: Methods and Application (P Herdewijn, ed., 2010) Humana Press, Oxford, whole of text;

(iv) Nucleic Acid Hybridization: A Practical Approach (B. D. Hames & S. J. Higgins, eds., 1985) IRL Press, Oxford, whole of text;

(v) van Pelt-Verkuil, E, van Belkum, A, Hays, J P. Principles and Technical Aspects of PCR Amplification (2010) Springer, whole of text;

(vi) Perbal, B., A Practical Guide to Molecular Cloning, 3rd Ed. (2008);

(vii) Gene Synthesis: Methods and Protocols (J Peccoud, ed. 2012) Humana Press, whole of text;

(viii) PCR Primer Design (Methods in Molecular Biology). (A Yuryev. ed., 2010), Humana Press, Oxford, whole of text.

Computer Embodiment

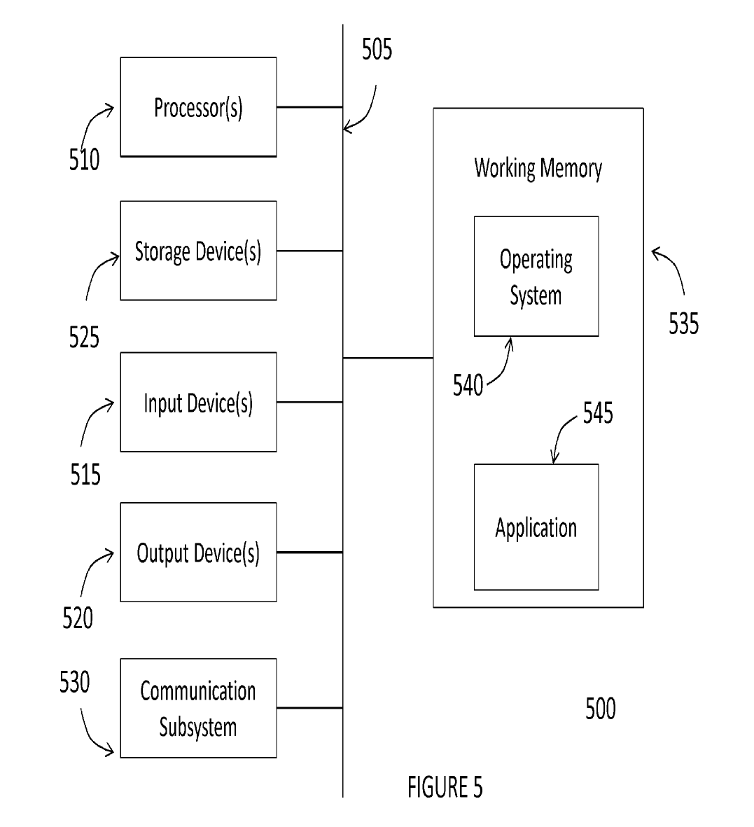

FIG. 5 provides a schematic illustration of one embodiment of a computer system 1500 that can perform the methods of the invention, as described herein. It should be noted that FIG. 5 is meant only to provide a generalized illustration of various components, any or all of which may be utilized as appropriate. FIG. 5, therefore, broadly illustrates how individual system elements may be implemented in a relatively separated or relatively more integrated manner.

The computer system 500 is shown comprising hardware elements that can be electrically coupled via a bus 505 (or may otherwise be in communication, as appropriate). The hardware elements can include one or more processors 510, including without limitation, one or more general purpose processors and/or one or more special purpose processors (such as digital signal processing chips, graphics acceleration chips, and/or the like); one or more input devices 515, which can include without limitation a mouse, a keyboard and/or the like; and one or more output devices 520, which can include without limitation a display device, a printer and/or the like.

The computer system 500 may further include (and/or be in communication with) one or more storage devices 525, which can comprise, without limitation, local and/or network accessible storage and/or can include, without limitation, a disk drive, a drive array, an optical storage device, a solid state storage device such as a random access memory (“RAM”) and/or a read-only memory (“ROM”), which can be programmable, flash updateable and/or the like. The computer system 500 might also include a communications subsystem 530, which can include without limitation a modem, a network card (wireless or wired), an infrared communication device, a wireless communication device and/or chipset (such as a Bluetooth™ device, an 802.11 device, a WiFi device, a WiMax device, cellular communication facilities, etc.), and/or the like. The communications subsystem 530 may permit data to be exchanged with a network (such as the network described below, to name one example), and/or any other devices described herein. In many embodiments, the computer system 500 will further comprise a working memory 535, which can include a RAM or ROM device, as described above.

The computer system 500 also can comprise software elements, shown as being currently located within the working memory 535, including an operating system 540 and/or other code, such as one or more application programs 545, which may comprise computer programs of the invention, and/or may be designed to implement methods of the invention and/or configure systems of the invention, as described herein. Merely by way of example, one or more procedures described with respect to the method(s) discussed above might be implemented as code and/or instructions executable by a computer (and/or a processor within a computer). A set of these instructions and/or codes might be stored on a computer-readable storage medium, such as the storage device(s) 525 described above. In some cases, the storage medium might be incorporated within a computer system, such as the system 500. In other embodiments, the storage medium might be separate from a computer system (i.e., a removable medium, such as a compact disc, etc.), and is provided in an installation package, such that the storage medium can be used to program a general-purpose computer with the instructions/code stored therein. These instructions might take the form of executable code, which is executable by the computer system 500 and/or might take the form of source and/or installable code, which, upon compilation and/or installation on the computer system 500 (e.g., using any of a variety of generally available compilers, installation programs, compression/decompression utilities, etc.), then takes the form of executable code.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

In one aspect, the invention employs a computer system (such as the computer system 500) to perform methods of the invention. According to a set of embodiments, some or all of the procedures of such methods are performed by the computer system 500 in response to processor 510 executing one or more sequences of one or more instructions (which might be incorporated into the operating system 540 and/or other code, such as an application program 545) contained in the working memory 535. Such instructions may be read into the working memory 535 from another machine-readable medium, such as one or more of the storage device(s) 525. Merely by way of example, execution of the sequences of instructions contained in the working memory 535 might cause the processor(s) 510 to perform one or more procedures of the methods described herein.

The terms “machine-readable medium” and “computer readable medium,” as used herein, refer to any medium that participates in providing data that causes a machine to operate in a specific fashion. In an embodiment implemented using the computer system 500, various machine-readable media might be involved in providing instructions/code to processor(s) 510 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media includes, for example, optical or magnetic disks, such as the storage device(s) 525. Volatile media includes, without limitation, dynamic memory, such as the working memory 535. Transmission media includes coaxial cables, copper wire and fiber optics, including the wires that comprise the bus 505, as well as the various components of the communications subsystem 530 (and/or the media by which the communications subsystem 530 provides communication with other devices). Hence, transmission media can also take the form of waves (including without limitation radio, acoustic and/or light waves, such as those generated during radio wave and infrared data communications).

Common forms of physical and/or tangible computer-readable media include, for example, a floppy disk, a flexible disk, a hard disk, magnetic tape, or any other magnetic medium, a CD-ROM, any other optical medium, punchcards, papertape, any other physical medium with patterns of holes, a RAM, a PROM, an EPROM, a FLASH-EPROM, any other memory chip or cartridge, a carrier wave as described hereinafter, or any other medium from which a computer can read instructions and/or code.

Various forms of machine-readable media may be involved in carrying one or more sequences of one or more instructions to the processor(s) 510 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 500. These signals, which might be in the form of electromagnetic signals, acoustic signals, optical signals and/or the like, are all examples of carrier waves on which instructions can be encoded, in accordance with various embodiments of the invention.

The communications subsystem 530 (and/or components thereof) generally will receive the signals, and the bus 505 then might carry the signals (and/or the data, instructions, etc., carried by the signals) to the working memory 535, from which the processor(s) 510 retrieves and executes the instructions. The instructions received by the working memory 535 may optionally be stored on a storage device 525 either before or after execution by the processor(s) 510.

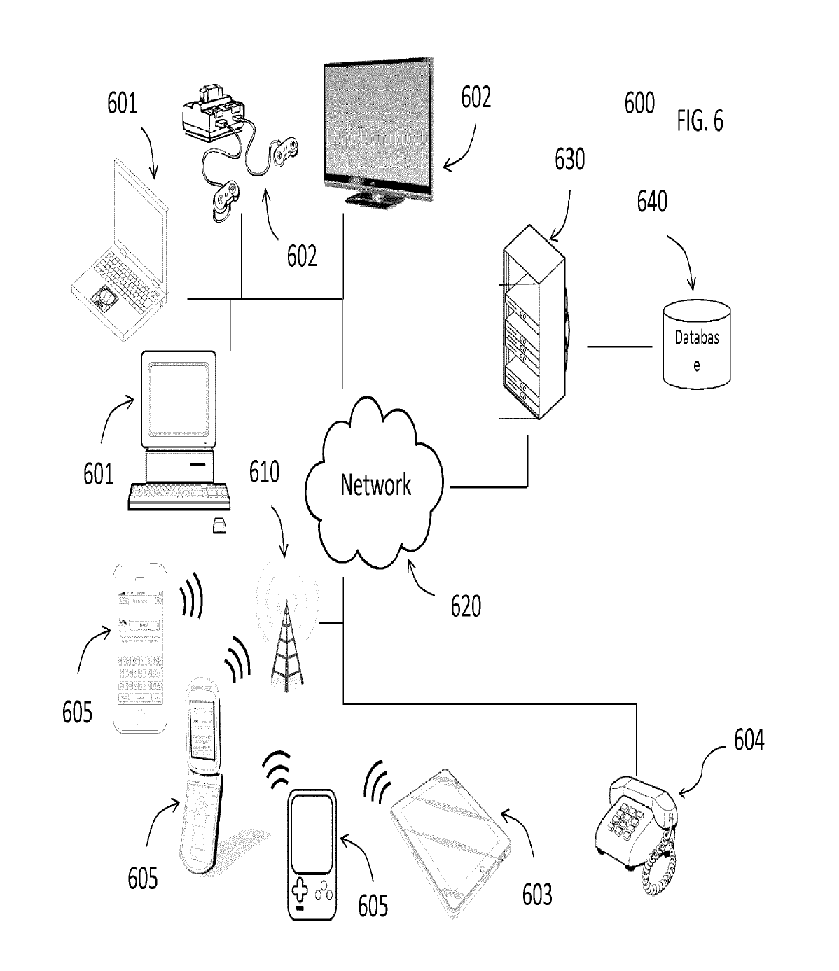

Merely by way of example, FIG. 6 illustrates a schematic diagram of devices to access and implement the invention system 600. The system 600 can include one or more user computers 601. The user computers 601 can be general-purpose personal computers (including, merely by way of example, personal computers and/or laptop computers running any appropriate flavor of Microsoft Corp.'s Windows™ and/or Apple Corp.'s Macintosh™ operating systems) and/or workstation computers running any of a variety of commercially available UNIX™ or UNIX-like operating systems. These user computers 601 can also have any of a variety of applications, including one or more applications configured to perform methods of the invention, as well as one or more office applications, database client and/or server applications, and web browser applications. Alternatively, the user computers 601 can be any other electronic device, such as a thin-client computer, media computing platforms 602 (e.g., gaming platforms, or cable and satellite set top boxes with navigation and recording capabilities), handheld computing devices (e.g., PDAs, tablets or handheld gaming platforms) 603, conventional land lines 604 (wired and wireless), mobile (e.g., cell or smart) phones 605 or tablets, or any other type of portable communication or computing platform (e.g., vehicle navigation systems), capable of communicating via a network (e.g., the network 620 described below) and/or displaying and navigating web pages or other types of electronic documents. Although the exemplary system 600 is shown with a user computer 601, any number of user computers can be supported.

Certain embodiments of the invention operate in a networked environment, which can include a network 620. The network 620 can be any type of network familiar to those skilled in the art that can support data communications using any of a variety of commercially available protocols, including without limitation TCP/IP, SNA, IPX, AppleTalk, and the like. Merely by way of example, the network 620 can be a local area network (“LAN”), including without limitation an Ethernet network, a Token-Ring network and/or the like; a wide-area network (WAN); a virtual network, including without limitation a virtual private network (“VPN”); the Internet; an intranet; an extranet; a public switched telephone network (“PSTN”); an infrared network; a wireless network 610, including without limitation a network operating under any of the IEEE 802.11 suite of protocols, the Bluetooth™ protocol known in the art, and/or any other wireless protocol 610; and/or any combination of these and/or other networks.

Embodiments of the invention can include one or more server computers 630. Each of the server computers 630 may be configured with an operating system, including without limitation any of those discussed above, as well as any commercially (or freely) available server operating systems. Each of the servers 630 may also be running one or more applications, which can be configured to provide services to one or more clients and/or other servers.

Merely by way of example, one of the servers 630 may be a web server, which can be used, merely by way of example, to process requests for web pages or other electronic documents from user computers 601. The web server can also run a variety of server applications, including HTTP servers, FTP servers, CGI servers, database servers, Java™ servers, and the like. In some embodiments of the invention, the web server may be configured to serve web pages that can be operated within a web browser on one or more of the user computers 601 to perform methods of the invention.

The server computers 630, in some embodiments, might include one or more application servers, which can include one or more applications accessible by a client running on one or more of the client computers and/or other servers. Merely by way of example, the server(s) 630 can be one or more general purpose computers capable of executing programs or scripts in response to the user computers and/or other servers, including without limitation web applications (which might, in some cases, be configured to perform methods of the invention). Merely by way of example, a web application can be implemented as one or more scripts or programs written in any suitable programming language, such as Java™, C, C#™ or C++, and/or any scripting language, such as Perl, Python, or TCL, as well as combinations of any programming/scripting languages. The application server(s) can also include database servers, including without limitation those commercially available from Oracle™, Microsoft™, Sybase™, IBM™ and the like, which can process requests from clients (including, depending on the configuration, database clients, API clients, web browsers, etc.) running on a user computer and/or another server. In some embodiments, an application server can create web pages dynamically for displaying the information in accordance with embodiments of the invention. Data provided by an application server may be formatted as web pages (comprising HTML, Javascript, etc., for example) and/or may be forwarded to a user computer via a web server (as described above, for example). Similarly, a web server might receive web page requests and/or input data from a user computer and/or forward the web page requests and/or input data to an application server. In some cases a web server may be integrated with an application server.

In accordance with further embodiments, one or more servers 630 can function as a file server and/or can include one or more of the files (e.g., application code, data files, etc.) necessary to implement methods of the invention incorporated by an application running on a user computer and/or another server. Alternatively, as those skilled in the art will appreciate, a file server can include all necessary files, allowing such an application to be invoked remotely by a user computer and/or server. It should be noted that the functions described with respect to various servers herein (e.g., application server, database server, web server, file server, etc.) can be performed by a single server and/or a plurality of specialized servers, depending on implementation-specific needs and parameters.

In certain embodiments, the system can include one or more databases 640. The location of the database(s) 640 is discretionary. Merely by way of example, a database might reside on a storage medium local to (and/or resident in) a server (and/or a user computer). Alternatively, a database can be remote from any or all of the computers, so long as the database can be in communication (e.g., via the network) with one or more of these. In a particular set of embodiments, a database can reside in a storage-area network (“SAN”) familiar to those skilled in the art. (Likewise, any necessary files for performing the functions attributed to the computers can be stored locally on the respective computer and/or remotely, as appropriate.) In one set of embodiments, the database can be a relational database, such as an Oracle™ database, that is adapted to store, update, and retrieve data in response to SQL-formatted commands. The database might be controlled and/or maintained by a database server, as described above, for example.

While the invention has been particularly shown and described with reference to specific embodiments thereof, it will be understood by those skilled in the art that changes in the form and details of the disclosed embodiments may be made without departing from the spirit or scope of the invention. For example, embodiments have been described herein with reference to the use of conventional landlines and cellular phones. Additionally, the various embodiments of the invention as described may be implemented in the form of software running on a general purpose computer, in the form of a specialized hardware, or combination of software and hardware. It will be understood, however, that the invention is not so limited. That is, embodiments are contemplated in which a much wider diversity of communication devices may be employed in various combinations to effect redemption.

In addition, although various advantages, aspects, and objects of the present invention have been discussed herein with reference to various embodiments, it will be understood that the scope of the invention should not be limited by reference to such advantages, aspects, and objects. Rather, the scope of the invention should be determined with reference to the appended claims.

### EXAMPLE

Exemplary reports and assessments are attached hereto as attachments.

| Gene | rsID | Marker info | Drug | Category |
|---|---|---|---|---|
| ABCB1 | rs1045642 | | nortriptyline | Toxicity/ADR |
| ABCB1 | rs1128503 | | risperidone | Efficacy |
| ABCB1 | rs2032582 | ABCB1:2677G > T/A, | Paroxetine | May have improved |
| | | Ala893Ser/Thr | | response |
| ABCB1 | rs2032583 | | antidepressants, | Efficacy |
| | | | Other | |
| | | | antidepressants | |
| ABCB1 | rs2229109 | | prazosin | Other |
| ABCB1 | rs2235015 | | antidepressants, | Efficacy |
| | | | Other | |
| | | | antidepressants | |
| ABCB1 | rs72552784 | | prazosin | Other |
| ABCB1 | rs9282564 | | prazosin | Other |
| ABCC2 | rs2273697 | | carbamazepine | Toxicity/ADR |
| ADM, | rs11042725 | | paroxetine | Efficacy |
| SBF2 | | | | |
| ADRB3 | rs4993 | | | A review of antipsychotic- |
| | | | | induced weight gain |
| ADRB3 | rs4994 | ADRB3:Trp64Arg | Olanzapine | Less likely to gain weight |
| AKT1 | rs2494732 | | risperidone | Efficacy |
| ANKK1 | rs1800497 | | antipsychotics | Toxicity/ADR |
| ARVCF, | rs165599 | | bupropion, | Efficacy |
| COMT | | | risperidone | |
| ASTN2 | rs4838255 | | | GWAS on metabolic side |
| | | | | effects of antipsychotics |
| ATF7IP2 | rs13335336 | | | GWAS on metabolic side |
| | | | | effects of antipsychotics |
| BAT2, | rs750332 | | carbamazepine | Toxicity/ADR |
| BAT3 | | | | |
| BDNF | rs61888800 | | Desipramine; | Depression may improve |
| | | | Fluoxetine | more than average |
| BDNF | rs6265 | | Psych panel | expanded PGx |
| | | | (Kelso request) | |
| BRUNOL4 | rs4799915 | | Iloperidone | Likely increased risk for QT |
| | | | | prolongation |
| CACNA1C | rs1006737 | | Genomind | expanded PGx |
| CACNG2 | rs2284017 | | Lithium | Efficacy |
| CACNG2 | rs5750285 | | Lithium | Increased likelihood of |
| | | | | response |
| CDH13 | rs17216786 | | | GWAS on metabolic side |
| | | | | effects of antipsychotics |
| CERKL | rs993648 | | Iloperidone | Likely decreased risk for QT |
| | | | | prolongation |
| CLCN6, | rs1801133 | | antipsychotics | Toxicity/ADR |
| MTHFR | | | | |
| CLMN | rs1187614 | | | GWAS on metabolic side |
| | | | | effects of antipsychotics |
| CNTF | rs1800169 | CNTF: FS63TER | Iloperidone | More likely to respond |
| COMT | rs4680 | | Modafinil | expanded PGx |
| CREB1, | rs7569963 | | citalopram | Efficacy, Toxicity/ADR |
| FAM119A | | | | |
| CYP1A2 | rs12720461 | *1K (−729C > T) | Psych panel | expanded PGx |
| | | | (Kelso request), | |
| | | | −729C > T | |
| CYP1A2 | rs2069514 | *1C | Psych panel | expanded PGx |
| | | | (Kelso request), | |
| | | | Genelex | |
| CYP1A2 | rs35694136 | *1D | Psych panel | expanded PGx |
| | | | (Kelso request), | |
| | | | −2467delT, | |
| | | | conflicting data | |
| | | | on functional | |
| | | | change | |
| CYP1A2 | rs762551 | | | current reports |
| CYP2B6 | rs2279343 | *4 | Efavirenz, | expanded PGx |
| | | | Methadone, | |
| | | | Buproprion | |
| CYP2B6 | rs28399499 | *18 | Efavirenz, | expanded PGx |
| | | | Methadone, | |
| | | | Buproprion | |
| CYP2B6 | rs3211371 | *5 | Efavirenz, | expanded PGx |
| | | | Methadone, | |
| | | | Buproprion | |
| CYP2B6 | rs3745274 | *9 | Efavirenz, | expanded PGx |
| | | | Methadone, | |
| | | | Buproprion | |
| CYP2B6 | rs8192709 | *2 | | expanded PGx |
| CYP2C19 | rs12248560 | *17 | | Medco single-test pgx |
| | | | | request |
| CYP2C19 | rs28399504 | *4 | | Medco single-test pgx |
| | | | | request |
| CYP2C19 | rs41291556 | *8 | | Medco single-test pgx |
| | | | | request |
| CYP2C19 | rs4244285 | *2 | | current reports |
| CYP2C19 | rs4986893 | *3 | | current reports |
| CYP2C19 | rs56337013 | *5 | | Medco single-test pgx |
| | | | | request |
| CYP2C19 | rs72552267 | *6 | | Medco single-test pgx |
| | | | | request |
| CYP2C19 | rs72558186 | *7 | | Medco single-test pgx |
| | | | | request |
| CYP2C9 | rs1057910 | *3 | | current reports |
| CYP2C9 | rs1799853 | *2 | | current reports |
| CYP2C9 | rs28371685 | *11 | | current reports |
| CYP2C9 | rs28371686 | *5 | | 2C9 panel |
| CYP2C9 | rs7089580 | 18786 | | 2C9 panel |
| CYP2C9 | rs9332131 | *6 | | 2C9 panel |
| CYP2D6 | rs1065852 | *10 | | cardio beta blockers |
| CYP2D6 | rs1080985 | *2 | | cardio beta-blockers |
| CYP2D6 | rs16947 | *2 | | cardio beta-blockers |
| CYP2D6 | rs1800716 | | | |
| CYP2D6 | rs28371706 | *17 | | cardio beta blockers |
| CYP2D6 | rs28371725 | *41 | | cardio beta blockers |
| CYP2D6 | rs35742686 | *3 | | cardio beta blockers |
| CYP2D6 | rs3892097 | *4 | | cardio beta blockers |
| CYP2D6 | rs5030655 | *6 | | cardio beta blockers |
| CYP2D6 | rs5030656 | *9 | | cardio beta blockers |
| CYP2D6 | rs5030865 | *8 | | cardio beta-blockers |
| CYP2D6 | rs59421388 | *29 | | cardio beta blockers |
| CYP2D6 | rs769258 | *35 | | cardio beta-blockers |
| CYP2D6 | i6/e9/i/5F | *5 | | 2D6 CNV assays + controls |
| enhanced | | | | |
| CYP2D6 | i6/e9/i/5F | del/dup | | 2D6 CNV assays + controls |
| enhanced | | | | |
| CYP2D6 | i6/e9/i2 | *36, *36xN | | 2D6 CNV assays + controls |
| enhanced | | | | |
| CYP2D6 | ID from | *14? | | |
| enhanced | Cindy | | | |
| CYP2D6 | rs1065852 | *10, *10xN | | REL assay + GT assay |
| enhanced | | | | |
| CYP2D6 | rs1080985 | *2, *2xN | | REL assay + GT assay |
| enhanced | | | | |
| CYP2D6 | rs16947 | *2 | | |
| enhanced | | | | |
| CYP2D6 | rs28371706 | *17, *17xN | | REL assay + GT assay |
| enhanced | | | | |
| CYP2D6 | rs28371725 | *41, *41xN | | REL assay + GT assay |
| enhanced | | | | |
| CYP2D6 | rs35742686 | *3 | | on Cindy's list but not on |
| enhanced | | | | pgx list |
| CYP2D6 | rs3892097 | *4, *4xN | | REL assay + GT assay |
| enhanced | | | | |
| CYP2D6 | rs5030655 | *6, *6xN | | REL assay + GT assay |
| enhanced | | | | |
| CYP2D6 | rs5030656 | *9 | | |
| enhanced | | | | |
| CYP2D6 | rs5030862 | *12 | | on Cindy's list but not on |
| enhanced | | | | pgx list |
| CYP2D6 | rs5030863 | *11 | | on Cindy's list but not on |
| enhanced | | | | pgx list |
| CYP2D6 | rs5030865 | *8 | | triallele with *14 |
| enhanced | | | | |
| CYP2D6 | rs5030867 | *7 | | |
| enhanced | | | | |
| CYP2D6 | rs59421388 | *29 | | |
| enhanced | | | | |
| CYP2D6 | rs72549357 | *15 | | on Cindy's list but not on |
| enhanced | | | | pgx list |
| CYP2D6 | rs769258 | *35 | | |
| enhanced | | | | |
| CYP3A, | rs12721627 | | midazolam | Other |
| CYP3A4 | | | | |
| DRD2 | rs1079598 | | clozapine, olanzapine | Toxicity/ADR |
| DRD2 | rs1799732 | | Psych panel | expanded PGx |
| | | | (Kelso request), | |
| | | | Genomind | |
| DRD2 | rs1799978 | DRD2: −241A > G | Risperidone | May respond well |
| DRD2 | rs6277 | | clozapine, olanzapine | Toxicity/ADR |
| DRD3 | rs167771 | | risperidone | Toxicity/ADR |
| DRD3 | rs6280 | DRD3:SER9GLY | Olanzapine | Schizophrenia more likely to |
| | | | | improve |
| DTNBP1 | rs742105 | | clozapine | Efficacy |
| DTNBP1 | rs909706 | | clozapine, haloperidol | Efficacy |
| EPHX1 | rs2234922 | | carbamazepine | Other |
| FHOD3 | rs17651157 | | | GWAS on metabolic side |
| | | | | effects of antipsychotics |
| FKBP5 | rs1360780 | | antidepressants | Efficacy |
| FKBP5 | rs3800373 | | antidepressants | Efficacy |
| GNB3 | rs5443 | GNB3:825C > T | Olanzapine | More likely to gain weight |
| GPR98 | rs1967256 | | | GWAS on metabolic side |
| | | | | effects of antipsychotics |
| GRIA2 | rs9784453 | | Lithium | expanded PGx |
| GRIA3 | rs4825476 | | Citalopram | May increase risk of suicidal |
| | | | | ideation during therapy |
| GRIK4 | rs1954787 | | Citalopram | expanded PGx |
| GRM3 | rs724226 | | Risperidone | May respond well |
| GSK3B | rs334558 | | Psych panel | expanded PGx |
| | | | (Kelso request) | |
| HLA | rs3909184 | HLA-B*1502 | carbamazepine | current reports |
| HLA | rs2844682 | HLA-B*1502 | carbamazepine | current reports |
| HSPA1A | rs1043620 | HSPA1A +438 C/T | Carbamazepine | SNP is part of protective |
| | | | | haplotype for |
| | | | | hypersensitivity to |
| | | | | carbamazepine |
| HSPA1A, | rs2227956 | | carbamazepine | Toxicity/ADR |
| HSPA1L | | | | |
| HTR1A | rs10042486 | | fluvoxamine, milnacipran, | Efficacy |
| | | | paroxetine | |
| HTR1A | rs6295 | | antidepressants | Efficacy |
| HTR2A | rs6311 | | AssureRx | expanded PGx |
| HTR2A | rs6313 | HTR2A:T102C | Olanzapine | More likely to gain weight |
| HTR2A | rs7997012 | | Psych panel | expanded PGx |
| | | | (Kelso request) | |
| HTR2C | rs1414334 | | antipsychotics, clozapine, | Toxicity/ADR |
| | | | risperidone | |
| HTR2C | rs3813928 | | risperidone | Efficacy |
| HTR2C | rs3813929 | HTR2C: −759C/T | Olanzapine | More likely to gain weight |
| HTR2C | rs518147 | HTR2C: −697G/C | Olanzapine | Less likely to gain weight |
| HTR2C | rs6318 | HTR2C:Cys23Ser | Olanzapine | More likely to gain weight |
| Intergenic | rs10202231 | | | GWAS on metabolic side |
| | | | | effects of antipsychotics |
| Intergenic | rs10499504 | | | GWAS on metabolic side |
| | | | | effects of antipsychotics |
| Intergenic | rs11163585 | | | GWAS on metabolic side |
| | | | | effects of antipsychotics |
| Intergenic | rs1117324 | | | GWAS on metabolic side |
| | | | | effects of antipsychotics |
| Intergenic | rs11663206 | | | GWAS on metabolic side |
| | | | | effects of antipsychotics |
| Intergenic | rs11735070 | | | GWAS on metabolic side |
| | | | | effects of antipsychotics |
| Intergenic | rs1405687 | | | GWAS on metabolic side |
| | | | | effects of antipsychotics |
| Intergenic | rs1534238 | | | GWAS on metabolic side |
| | | | | effects of antipsychotics |
| Intergenic | rs1577917 | | | GWAS on metabolic side |
| | | | | effects of antipsychotics |
| Intergenic | rs17100498 | | | GWAS on metabolic side |
| | | | | effects of antipsychotics |
| Intergenic | rs17385675 | | | GWAS on metabolic side |
| | | | | effects of antipsychotics |
| Intergenic | rs17410015 | | | GWAS on metabolic side |
| | | | | effects of antipsychotics |
| Intergenic | rs17661538 | | | GWAS on metabolic side |
| | | | | effects of antipsychotics |
| Intergenic | rs2994684 | | | GWAS on metabolic side |
| | | | | effects of antipsychotics |
| Intergenic | rs320209 | | | GWAS on metabolic side |
| | | | | effects of antipsychotics |
| Intergenic | rs399885 | | | GWAS on metabolic side |
| | | | | effects of antipsychotics |
| Intergenic | rs4783227 | | | GWAS on metabolic side |
| | | | | effects of antipsychotics |
| Intergenic | rs518590 | | | GWAS on metabolic side |
| | | | | effects of antipsychotics |
| Intergenic | rs6092078 | | | GWAS on metabolic side |
| | | | | effects of antipsychotics |
| Intergenic | rs6735179 | | | GWAS on metabolic side |
| | | | | effects of antipsychotics |
| Intergenic | rs7105881 | | | GWAS on metabolic side |
| | | | | effects of antipsychotics |
| Intergenic | rs7570469 | | | GWAS on metabolic side |
| | | | | effects of antipsychotics |
| Intergenic | rs8092443 | | | GWAS on metabolic side |
| | | | | effects of antipsychotics |
| Intergenic | rs977396 | | | GWAS on metabolic side |
| | | | | effects of antipsychotics |
| KIRREL3 | rs620875 | | | GWAS on metabolic side |
| | | | | effects of antipsychotics |
| LEP | rs4731426 | | olanzapine | Toxicity/ADR |
| LEP | rs7799039 | | risperidone | Toxicity/ADR |
| LEPR | rs817983 | | | A review of antipsychotic- |
| | | | | induced weight gain |
| LOC729993 | rs153091 | | | GWAS on metabolic side |
| | | | | effects of antipsychotics |
| LTA, TNF | rs1800629 | | carbamazepine | Toxicity/ADR |
| MC4R | rs8087522 | | clozapine | clozapine-induced weight |
| | | | | gain |
| MEIS2 | rs1568679 | | | GWAS on metabolic side |
| | | | | effects of antipsychotics |
| NR3C1 | rs10482633 | | Escitalopram; | Depression may not respond |
| | | | Nortriptyline | as well |
| NRG3 | rs4933824 | | Iloperidone | Likely increased risk for QT |
| | | | | prolongation |
| NTRK2 | rs10868235 | | Lithium | expanded PGx |
| NTRK2 | rs1387923 | | Lithium | expanded PGx |
| NUBPL | rs7142881 | | Iloperidone | Likely increased risk for QT |
| | | | | prolongation |
| OPRM1 | rs1799971 | | Naltrexone, | expanded PGx |
| | | | morphine | |
| PALLD | rs17054392 | | Iloperidone | Likely increased risk for QT |
| | | | | prolongation |
| PMCH | rs7973796 | | | A review of antipsychotic- |
| | | | | induced weight gain |
| PPARD | rs9658108 | | | GWAS on metabolic side |
| | | | | effects of antipsychotics |
| PRKAA1 | rs10074991 | | | A review of antipsychotic- |
| | | | | induced weight gain |
| PRKAR2B | rs13224682 | | | GWAS on metabolic side |
| | | | | effects of antipsychotics |
| RGS4 | rs10917670 | | Risperidone | May not respond well |
| RGS4 | rs2661319 | | Risperidone | May not respond well |
| RGS4 | rs2842030 | | perphenazine, | Efficacy |
| | | | risperidone | |
| RGS4 | rs951439 | | Risperidone | May not respond well |
| RNF144A | rs6741819 | | | GWAS on metabolic side |
| | | | | effects of antipsychotics |
| SCN1A | rs3812718 | | carbamazepine | Dosage |
| SERPINE1 | rs1799889 | | antidepressants, | Efficacy |
| | | | citalopram, fluoxetine | |
| SERPINE1 | rs2227631 | | antidepressants, | Efficacy |
| | | | citalopram, fluoxetine | |
| SLC6A4 | none | 5-HTTLPR | Psych panel | expanded PGx |
| | | | (Kelso request), | |
| | | | Genomind, | |
| | | | AssureRx | |
| SLC6A4 | rs25531 | | Genomind | expanded PGx |
| SLC6A4 | rs4795541 | | escitalopram | Efficacy, Toxicity/ADR |
| SLCO3A1 | rs3924426 | | Iloperidone | Likely increased risk for QT |
| | | | | prolongation |
| SOX5 | rs1464500 | | | GWAS on metabolic side |
| | | | | effects of antipsychotics |
| TPH2 | rs10879346 | | antidepressants, | Efficacy |
| | | | mirtazapine, venlafaxine | |
| TPH2 | rs1487278 | | mirtazapine, venlafaxine | Efficacy |
| UGT2B15 | rs1902023 | | Benzodiazepines | expanded PGx |
| | | | (diazepam) | |
| ZBTB42 | rs3803300 | | risperidone | Efficacy |
| SLC6A3 | rs37020 | | stimulants | |
| SLC6A3 | rs460000 | | bupropion | |
| CREB1 | rs6740584 | | | risk for major depression |

| Gene | rsID | Source | Phenotype |
|---|---|---|---|
| ABCB1 | rs1045642 | PharmGKB list of Clinical | nortriptyline |
| | | Annotations May 11, 2012 | Toxicity/ADR |
| ABCB1 | rs1128503 | PharmGKB list of Clinical | risperidone |
| | | Annotations May 11, 2012 | Efficacy |
| ABCB1 | rs2032582 | PMID 20435227 Suppl Table 3a | Paroxetine, |
| | | | response |
| ABCB1 | rs2032583 | PharmGKB list of Clinical | antidepressants |
| | | Annotations May 11, 2012 | Efficacy |
| ABCB1 | rs2229109 | PharmGKB list of Clinical | prazosin |
| | | Annotations May 11, 2012 | metabolis |
| ABCB1 | rs2235015 | PharmGKB list of Clinical | antidepressants |
| | | Annotations May 11, 2012 | Efficacy |
| ABCB1 | rs72552784 | PharmGKB list of Clinical | prazosin |
| | | Annotations May 11, 2012 | metabolism |
| ABCB1 | rs9282564 | PharmGKB list of Clinical | prazosin |
| | | Annotations May 11, 2012 | metabolism |
| ABCC2 | rs2273697 | PharmGKB list of Clinical | carbamazepine |
| | | Annotations May 11, 2012 | Toxicity/ADR |
| ADM, | rs11042725 | PharmGKB list of Clinical | paroxetine |
| SBF2 | | Annotations May 11, 2012 | Efficacy |
| ADRB3 | rs4993 | PMID 21894153 | antipsychotic- |
| | | | induced weight |
| | | | gain |
| ADRB3 | rs4994 | PMID 20435227 Suppl Table 3c | Olanzapine, |
| | | | weight gain |
| AKT1 | rs2494732 | PharmGKB list of Clinical | risperidone |
| | | Annotations May 11, 2012 | Efficacy |
| ANKK1 | rs1800497 | PharmGKB list of Clinical | antipsychotics |
| | | Annotations May 11, 2012 | Toxicity/ADR |
| ARVCF, | rs165599 | PharmGKB list of Clinical | bupropion, |
| COMT | | Annotations May 11, 2012 | risperidone |
| | | | Efficacy |
| ASTN2 | rs4838255 | PMID 20195266 | metabolic side |
| | | | effects of |
| | | | antipsychotics |
| ATF7IP2 | rs13335336 | PMID 20195266 | metabolic side |
| | | | effects of |
| | | | antipsychotics |
| BAT2, | rs750332 | PharmGKB list of Clinical | carbamazepine |
| BAT3 | | Annotations May 11, 2012 | Toxicity/ADR |
| BDNF | rs61888800 | PMID 20435227 Suppl Table 3a | Desipramine, |
| | | | Fluoxetine, |
| | | | depression |
| | | | improvement |
| BDNF | rs6265 | F6 marker panel_12Apr12 | Lithium Efficacy |
| BRUNOL4 | rs4799915 | PMID 20435227 Suppl Table 3c | Iloperidone, risk |
| | | | for QT |
| | | | prolongation |
| CACNA1C | rs1006737 | F6 marker panel_12Apr12 | schizophrenia |
| CACNG2 | rs2284017 | PharmGKB list of Clinical | Lithium Efficacy |
| | | Annotations May 11, 2012 | |
| CACNG2 | rs5750285 | PMID 20435227 Suppl Table 3a | Lithium, |
| | | | response |
| CDH13 | rs17216786 | PMID 20195266 | metabolic side |
| | | | effects of |
| | | | antipsychotics |
| CERKL | rs993648 | PMID 20435227 Suppl Table 3c | Iloperidone, risk |
| | | | for QT |
| | | | prolongation |
| CLCN6, | rs1801133 | PharmGKB list of Clinical | antipsychotics |
| MTHFR | | Annotations May 11, 2012 | Toxicity/ADR |
| CLMN | rs1187614 | PMID 20195266 | metabolic side |
| | | | effects of |
| | | | antipsychotics |
| CNTF | rs1800169 | PMID 20435227 Suppl Table 3c | Iloperidone, |
| | | | response |
| COMT | rs4680 | F6 marker panel_12Apr12 | response to |
| | | | modafinil |
| CREB1, | rs7569963 | PharmGKB list of Clinical | citalopram |
| FAM119A | | Annotations May 11, 2012 | Efficacy, Toxicity/ |
| | | | ADR |
| CYP1A2 | rs12720461 | F6 marker panel_12Apr12 | metabolism of |
| | | | many drugs |
| CYP1A2 | rs2069514 | F6 marker panel_12Apr12 | metabolism of |
| | | | many drugs |
| CYP1A2 | rs35694136 | F6 marker panel_12Apr12 | metabolism of |
| | | | many drugs |
| CYP1A2 | rs762551 | F5 marker panel_updated | metabolism of |
| | | 17Apr12 | many drugs |
| CYP2B6 | rs2279343 | F6 marker panel_12Apr12 | Buproprion |
| | | | metabolism |
| CYP2B6 | rs28399499 | F6 marker panel_12Apr12 | Buproprion |
| | | | metabolism |
| CYP2B6 | rs3211371 | F6 marker panel_12Apr12 | Buproprion |
| | | | metabolism |
| CYP2B6 | rs3745274 | F6 marker panel_12Apr12 | Buproprion |
| | | | metabolism |
| CYP2B6 | rs8192709 | F6 marker panel_12Apr12 | Buproprion |
| | | | metabolism |
| CYP2C19 | rs12248560 | F5 marker panel_updated | metabolism of |
| | | 17Apr12 | many drugs |
| CYP2C19 | rs28399504 | F5 marker panel_updated | metabolism of |
| | | 17Apr12 | many drugs |
| CYP2C19 | rs41291556 | F5 marker panel_updated | metabolism of |
| | | 17Apr12 | many drugs |
| CYP2C19 | rs4244285 | F5 marker panel_updated | metabolism of |
| | | 17Apr12 | many drugs |
| CYP2C19 | rs4986893 | F5 marker panel_updated | metabolism of |
| | | 17Apr12 | many drugs |
| CYP2C19 | rs56337013 | F5 marker panel_updated | metabolism of |
| | | 17Apr12 | many drugs |
| CYP2C19 | rs72552267 | F5 marker panel_updated | metabolism of |
| | | 17Apr12 | many drugs |
| CYP2C19 | rs72558186 | F5 marker panel_updated | metabolism of |
| | | 17Apr12 | many drugs |
| CYP2C9 | rs1057910 | F5 marker panel_updated | metabolism of |
| | | 17Apr12 | many drugs |
| CYP2C9 | rs1799853 | F5 marker panel_updated | metabolism of |
| | | 17Apr12 | many drugs |
| CYP2C9 | rs28371685 | F5 marker panel_updated | metabolism of |
| | | 17Apr12 | many drugs |
| CYP2C9 | rs28371686 | F5 marker panel_updated | metabolism of |
| | | 17Apr12 | many drugs |
| CYP2C9 | rs7089580 | F5 marker panel_updated | metabolism of |
| | | 17Apr12 | many drugs |
| CYP2C9 | rs9332131 | F5 marker panel_updated | metabolism of |
| | | 17Apr12 | many drugs |
| CYP2D6 | rs1065852 | F5 marker panel_updated | metabolism of |
| | | 17Apr12 | many drugs |
| CYP2D6 | rs1080985 | F5 marker panel_updated | metabolism of |
| | | 17Apr12 | many drugs |
| CYP2D6 | rs16947 | F5 marker panel_updated | metabolism of |
| | | 17Apr12 | many drugs |
| CYP2D6 | rs1800716 | PMID 20435227 Suppl Table 3a | metabolism of |
| | | | many drugs |
| CYP2D6 | rs28371706 | F5 marker panel_updated | metabolism of |
| | | 17Apr12 | many drugs |
| CYP2D6 | rs28371725 | F5 marker panel_updated | metabolism of |
| | | 17Apr12 | many drugs |
| CYP2D6 | rs35742686 | F5 marker panel_updated | metabolism of |
| | | 17Apr12 | many drugs |
| CYP2D6 | rs3892097 | F5 marker panel_updated | metabolism of |
| | | 17Apr12 | many drugs |
| CYP2D6 | rs5030655 | F5 marker panel_updated | metabolism of |
| | | 17Apr12 | many drugs |
| CYP2D6 | rs5030656 | F5 marker panel_updated | metabolism of |
| | | 17Apr12 | many drugs |
| CYP2D6 | rs5030865 | F5 marker panel_updated | metabolism of |
| | | 17Apr12 | many drugs |
| CYP2D6 | rs59421388 | F5 marker panel_updated | metabolism of |
| | | 17Apr12 | many drugs |
| CYP2D6 | rs769258 | F5 marker panel_updated | metabolism of |
| | | 17Apr12 | many drugs |
| CYP2D6 | i6/e9/i/5F | F4 Marker Panel 10Apr12 | metabolism of |
| enhanced | | | many drugs |
| CYP2D6 | i6/e9/i/5F | F4 Marker Panel 10Apr12 | metabolism of |
| enhanced | | | many drugs |
| CYP2D6 | i6/e9/i2 | F4 Marker Panel 10Apr12 | metabolism of |
| enhanced | | | many drugs |
| CYP2D6 | ID from | F4 Marker Panel 10Apr12 | metabolism of |
| enhanced | Cindy | | many drugs |
| CYP2D6 | rs1065852 | F4 Marker Panel 10Apr12 | metabolism of |
| enhanced | | | many drugs |
| CYP2D6 | rs1080985 | F4 Marker Panel 10Apr12 | metabolism of |
| enhanced | | | many drugs |
| CYP2D6 | rs16947 | F4 Marker Panel 10Apr12 | metabolism of |
| enhanced | | | many drugs |
| CYP2D6 | rs28371706 | F4 Marker Panel 10Apr12 | metabolism of |
| enhanced | | | many drugs |
| CYP2D6 | rs28371725 | F4 Marker Panel 10Apr12 | metabolism of |
| enhanced | | | many drugs |
| CYP2D6 | rs35742686 | F4 Marker Panel 10Apr12 | metabolism of |
| enhanced | | | many drugs |
| CYP2D6 | rs3892097 | F4 Marker Panel 10Apr12 | metabolism of |
| enhanced | | | many drugs |
| CYP2D6 | rs5030655 | F4 Marker Panel 10Apr12 | metabolism of |
| enhanced | | | many drugs |
| CYP2D6 | rs5030656 | F4 Marker Panel 10Apr12 | metabolism of |
| enhanced | | | many drugs |
| CYP2D6 | rs5030862 | F4 Marker Panel 10Apr12 | metabolism of |
| enhanced | | | many drugs |
| CYP2D6 | rs5030863 | F4 Marker Panel 10Apr12 | metabolism of |
| enhanced | | | many drugs |
| CYP2D6 | rs5030865 | F4 Marker Panel 10Apr12 | metabolism of |
| enhanced | | | many drugs |
| CYP2D6 | rs5030867 | F4 Marker Panel 10Apr12 | metabolism of |
| enhanced | | | many drugs |
| CYP2D6 | rs59421388 | F4 Marker Panel 10Apr12 | metabolism of |
| enhanced | | | many drugs |
| CYP2D6 | rs72549357 | F4 Marker Panel 10Apr12 | metabolism of |
| enhanced | | | many drugs |
| CYP2D6 | rs769258 | F4 Marker Panel 10Apr12 | metabolism of |
| enhanced | | | many drugs |
| CYP3A, | rs12721627 | PharmGKB list of Clinical | midazolam |
| CYP3A4 | | Annotations May 11, 2012 | metabolism |
| DRD2 | rs1079598 | PharmGKB list of Clinical | clozapine, olanzapine |
| | | Annotations May 11, 2012 | Toxicity/ADR |
| DRD2 | rs1799732 | F6 marker panel_12Apr12 | response to |
| | | | risperidone |
| DRD2 | rs1799978 | PMID 20435227 Suppl Table 3c | Risperidone, |
| | | | response |
| DRD2 | rs6277 | PharmGKB list of Clinical | clozapine, olanzapine |
| | | Annotations May 11, 2012 | Toxicity/ADR |
| DRD3 | rs167771 | PharmGKB list of Clinical | risperidone |
| | | Annotations May 11, 2012 | Toxicity/ADR |
| DRD3 | rs6280 | PMID 20435227 Suppl Table 3c | Olanzapine, |
| | | | Schizophrenia |
| | | | improvement |
| DTNBP1 | rs742105 | PharmGKB list of Clinical | clozapine Efficacy |
| | | Annotations May 11, 2012 | |
| DTNBP1 | rs909706 | PharmGKB list of Clinical | clozapine, haloperidol |
| | | Annotations May 11, 2012 | Efficacy |
| EPHX1 | rs2234922 | PharmGKB list of Clinical | carbamazepine |
| | | Annotations May 11, 2012 | metabolism |
| FHOD3 | rs17651157 | PMID 20195266 | metabolic side |
| | | | effects of |
| | | | antipsychotics |
| FKBP5 | rs1360780 | PharmGKB list of Clinical | antidepressants |
| | | Annotations May 11, 2012 | Efficacy |
| FKBP5 | rs3800373 | PharmGKB list of Clinical | antidepressants |
| | | Annotations May 11, 2012 | Efficacy |
| GNB3 | rs5443 | PMID 20435227 Suppl Table 3c | Olanzapine, |
| | | | weight gain |
| GPR98 | rs1967256 | PMID 20195266 | metabolic side |
| | | | effects of |
| | | | antipsychotics |
| GRIA2 | rs9784453 | F6 marker panel_12Apr12 | response to |
| | | | lithium |
| GRIA3 | rs4825476 | PMID 20435227 Suppl Table 3b | Citalopram, risk |
| | | | of suicidal |
| | | | ideation during |
| | | | therapy |
| GRIK4 | rs1954787 | F6 marker panel_12Apr12 | citalopram |
| | | | efficacy |
| GRM3 | rs724226 | PMID 20435227 Suppl Table 3c | Risperidone, |
| | | | response |
| GSK3B | rs334558 | F6 marker panel_12Apr12 | response to |
| | | | lithium |
| HLA | rs3909184 | F5 marker panel_updated | carbamazapine |
| | | 17Apr12 | hypersensitivity |
| HLA | rs2844682 | F5 marker panel_updated | carbamazapine |
| | | 17Apr12 | hypersensitivity |
| HSPA1A | rs1043620 | PMID 20435227 Suppl Table 3a | Carbamazepine, |
| | | | SNP is part of |
| | | | protective |
| | | | haplotype for |
| | | | hypersensitivity |
| | | | to |
| | | | carbamazepine |
| HSPA1A, | rs2227956 | PharmGKB list of Clinical | carbamazepine |
| HSPA1L | | Annotations May 11, 2012 | Toxicity/ADR |
| HTR1A | rs10042486 | PharmGKB list of Clinical | fluvoxamine, milnacipran, |
| | | Annotations May 11, 2012 | paroxetine |
| | | | Efficacy |
| HTR1A | rs6295 | PharmGKB list of Clinical | antidepressants |
| | | Annotations May 11, 2012 | Efficacy |
| HTR2A | rs6311 | F6 marker panel_12Apr12 | SSRIs, ADR |
| HTR2A | rs6313 | PMID 20435227 Suppl Table 3c | Olanzapine, |
| | | | weight gain |
| HTR2A | rs7997012 | F6 marker panel_12Apr12 | response to SSRIs |
| HTR2C | rs1414334 | PharmGKB list of Clinical | antipsychotics, clozapine, |
| | | Annotations May 11, 2012 | risperidone |
| | | | Toxicity/ADR |
| HTR2C | rs3813928 | PharmGKB list of Clinical | risperidone |
| | | Annotations May 11, 2012 | Efficacy |
| HTR2C | rs3813929 | PMID 20435227 Suppl Table 3c | Olanzapine, |
| | | | weight gain |
| HTR2C | rs518147 | PMID 20435227 Suppl Table 3c | Olanzapine, |
| | | | weight gain |
| HTR2C | rs6318 | PMID 20435227 Suppl Table 3c | Olanzapine, |
| | | | weight gain |
| Intergenic | rs10202231 | PMID 20195266 | metabolic side |
| | | | effects of |
| | | | antipsychotics |
| Intergenic | rs10499504 | PMID 20195266 | metabolic side |
| | | | effects of |
| | | | antipsychotics |
| Intergenic | rs11163585 | PMID 20195266 | metabolic side |
| | | | effects of |
| | | | antipsychotics |
| Intergenic | rs1117324 | PMID 20195266 | metabolic side |
| | | | effects of |
| | | | antipsychotics |
| Intergenic | rs11663206 | PMID 20195266 | metabolic side |
| | | | effects of |
| | | | antipsychotics |
| Intergenic | rs11735070 | PMID 20195266 | metabolic side |
| | | | effects of |
| | | | antipsychotics |
| Intergenic | rs1405687 | PMID 20195266 | metabolic side |
| | | | effects of |
| | | | antipsychotics |
| Intergenic | rs1534238 | PMID 20195266 | metabolic side |
| | | | effects of |
| | | | antipsychotics |
| Intergenic | rs1577917 | PMID 20195266 | metabolic side |
| | | | effects of |
| | | | antipsychotics |
| Intergenic | rs17100498 | PMID 20195266 | metabolic side |
| | | | effects of |
| | | | antipsychotics |
| Intergenic | rs17385675 | PMID 20195266 | metabolic side |
| | | | effects of |
| | | | antipsychotics |
| Intergenic | rs17410015 | PMID 20195266 | metabolic side |
| | | | effects of |
| | | | antipsychotics |
| Intergenic | rs17661538 | PMID 20195266 | metabolic side |
| | | | effects of |
| | | | antipsychotics |
| Intergenic | rs2994684 | PMID 20195266 | metabolic side |
| | | | effects of |
| | | | antipsychotics |
| Intergenic | rs320209 | PMID 20195266 | metabolic side |
| | | | effects of |
| | | | antipsychotics |
| Intergenic | rs399885 | PMID 20195266 | metabolic side |
| | | | effects of |
| | | | antipsychotics |
| Intergenic | rs4783227 | PMID 20195266 | metabolic side |
| | | | effects of |
| | | | antipsychotics |
| Intergenic | rs518590 | PMID 20195266 | metabolic side |
| | | | effects of |
| | | | antipsychotics |
| Intergenic | rs6092078 | PMID 20195266 | metabolic side |
| | | | effects of |
| | | | antipsychotics |
| Intergenic | rs6735179 | PMID 20195266 | metabolic side |
| | | | effects of |
| | | | antipsychotics |
| Intergenic | rs7105881 | PMID 20195266 | metabolic side |
| | | | effects of |
| | | | antipsychotics |
| Intergenic | rs7570469 | PMID 20195266 | metabolic side |
| | | | effects of |
| | | | antipsychotics |
| Intergenic | rs8092443 | PMID 20195266 | metabolic side |
| | | | effects of |
| | | | antipsychotics |
| Intergenic | rs977396 | PMID 20195266 | metabolic side |
| | | | effects of |
| | | | antipsychotics |
| KIRREL3 | rs620875 | PMID 20195266 | metabolic side |
| | | | effects of |
| | | | antipsychotics |
| LEP | rs4731426 | PharmGKB list of Clinical | olanzapine |
| | | Annotations May 11, 2012 | Toxicity/ADR |
| LEP | rs7799039 | PharmGKB list of Clinical | risperidone |
| | | Annotations May 11, 2012 | Toxicity/ADR |
| LEPR | rs817983 | PMID 21894153 | antipsychotic- |
| | | | induced weight |
| | | | gain |
| LOC729993 | rs153091 | PMID 20195266 | metabolic side |
| | | | effects of |
| | | | antipsychotics |
| LTA, TNF | rs1800629 | PharmGKB list of Clinical | carbamazepine |
| | | Annotations May 11, 2012 | Toxicity/ADR |
| MC4R | rs8087522 | PMID 22310352 | clozapine- |
| | | | induced weight |
| | | | gain |
| MEIS2 | rs1568679 | PMID 20195266 | metabolic side |
| | | | effects of |
| | | | antipsychotics |
| NR3C1 | rs10482633 | PMID 20435227 Suppl Table 3b | Escitalopram, |
| | | | Nortriptyline, |
| | | | response |
| NRG3 | rs4933824 | PMID 20435227 Suppl Table 3c | Iloperidone, risk |
| | | | for QT |
| | | | prolongation |
| NTRK2 | rs10868235 | F6 marker panel_12Apr12 | response to |
| | | | lithium |
| NTRK2 | rs1387923 | F6 marker panel_12Apr12 | response to |
| | | | lithium |
| NUBPL | rs7142881 | PMID 20435227 Suppl Table 3c | Iloperidone, risk |
| | | | for QT |
| | | | prolongation |
| OPRM1 | rs1799971 | F6 marker panel_12Apr12 | response to |
| | | | opioids |
| PALLD | rs17054392 | PMID 20435227 Suppl Table 3c | Iloperidone, risk |
| | | | for QT |
| | | | prolongation |
| PMCH | rs7973796 | PMID 21894153 | antipsychotic- |
| | | | induced weight |
| | | | gain |
| PPARD | rs9658108 | PMID 20195266 | metabolic side |
| | | | effects of |
| | | | antipsychotics |
| PRKAA1 | rs10074991 | PMID 21894153 | antipsychotic- |
| | | | induced weight |
| | | | gain |
| PRKAR2B | rs13224682 | PMID 20195266 | metabolic side |
| | | | effects of |
| | | | antipsychotics |
| RGS4 | rs10917670 | PMID 20435227 Suppl Table 3c | Risperidone, |
| | | | response |
| RGS4 | rs2661319 | PMID 20435227 Suppl Table 3c | Risperidone, |
| | | | response |
| RGS4 | rs2842030 | PharmGKB list of Clinical | perphenazine, |
| | | Annotations May 11, 2012 | risperidone |
| | | | Efficacy |
| RGS4 | rs951439 | PMID 20435227 Suppl Table 3c | Risperidone, |
| | | | response |
| RNF144A | rs6741819 | PMID 20195266 | metabolic side |
| | | | effects of |
| | | | antipsychotics |
| SCN1A | rs3812718 | PharmGKB list of Clinical | carbamazepine |
| | | Annotations May 11, 2012 | Dosage |
| SERPINE1 | rs1799889 | PharmGKB list of Clinical | antidepressants, citalopram, |
| | | Annotations May 11, 2012 | fluoxetine, |
| | | | Efficacy |
| SERPINE1 | rs2227631 | PharmGKB list of Clinical | antidepressants, citalopram, |
| | | Annotations May 11, 2012 | fluoxetine |
| | | | Efficacy |
| SLC6A4 | none | F6 marker panel_12Apr12 | response to SSRIs |
| SLC6A4 | rs25531 | F6 marker panel_12Apr12 | fluoxetine |
| | | | efficacy |
| SLC6A4 | rs4795541 | PharmGKB list of Clinical | escitalopram |
| | | Annotations May 11, 2012 | Efficacy, Toxicity/ |
| | | | ADR |
| SLCO3A1 | rs3924426 | PMID 20435227 Suppl Table 3c | Iloperidone, risk |
| | | | for QT |
| | | | prolongation |
| SOX5 | rs1464500 | PMID 20195266 | metabolic side |
| | | | effects of |
| | | | antipsychotics |
| TPH2 | rs10879346 | PharmGKB list of Clinical | antidepressants, |
| | | Annotations May 11, 2012 | mirtazapine, venlafaxine |
| | | | Efficacy |
| TPH2 | rs1487278 | PharmGKB list of Clinical | mirtazapine, venlafaxine |
| | | Annotations May 11, 2012 | Efficacy |
| UGT2B15 | rs1902023 | F6 marker panel_12Apr12 | oxazepam |
| | | | metabolism |
| ZBTB42 | rs3803300 | PharmGKB list of Clinical | risperidone |
| | | Annotations May 11, 2012 | Efficacy |
| SLC6A3 | rs37020 | Kelsoe list | |
| SLC6A3 | rs460000 | Kelsoe list | |
| CREB1 | rs6740584 | Kelsoe list | |

### Mental Health DNA Insight Content

| Phenotype Name | Gene | Outcome | Content |
|---|---|---|---|
| SSRIs | | | |
| Citalopram (RC) | CYP2C19 | Poor | Patient's genotype is associated with increased plasma concentrations of citalopram at standard doses [PMID |
| | | Metabolizer | 12968986, 16855453, 15168101, 12968986, 12975335]. Monitor patient for adverse effects [Celexa label, FDA Drug |
| | | | Safety Communication, PMID 21192344, 12968986, 16855453]. Decreased dosages are recommended [Celexa label, |
| | | | FDA Drug Safety Communication]. |
| Citalopram (RC) | CYP2C19 | Intermediate | Patient may have increased plasma concentrations of citalopram at standard doses [PMID 12968986, 16855453, |
| | | Metabolizer | 15168101, 12968986, 12975335, 16418702]. Monitor patient for adverse effects [PMID 21192344, 12968986, |
| | | | 16855453]. |
| Citalopram (RC) | CYP2C19 | Ultrarapid | Patient has decreased likelihood of responding to standard doses of citalopram (PMID 20531370, 21192344). Patient's |
| | | Metabolizer | genotype is associated with decreased plasma concentrations of citalopram at standard doses (PMID 20531370, |
| | | | 17625515, 18294333). Consider alternative medications, such as fluoxetine or paroxetine (PMID 21412232). |
| Citalopram (RC) | SLC6A4 | Decreased risk | Patient has decreased risk of adverse effects if treated with citalopram for major depressive disorder (PMID |
| [used “SLC6A4 | | of adverse | 18982004). |
| Reporting Strategy | | effects | |
| v4_0214a”] | | | |
| Citalopram (RC) | SLC6A4 | Increased risk | Patient has increased risk of adverse effects, such as headache, nausea, drowsiness, agitation, sexual dysfunction or |
| | | of adverse | weight gain, if treated with citalopram for major depressive disorder (PMID 18982004, |
| | | effects | http://www.nimh.nih.gov/health/publications/mental-health-medications/nimh-mental-health-medications.pdf). |
| Citalopram (RC) | HTR2A | Decreased risk | Patient's genotype is associated with decreased risk of nonresponse to treatment in Caucasians (No PMID in reporting |
| [used HTR2A SSRI | (rs7997012 | of nonresponse | strategy). |
| reporting strategy | AA) | to citalopram | |
| v1] | | treatment | |
| Citalopram (RC) | HTR2A | Increased risk | Patient's genotype is associated with increased risk of nonresponse to treatment in Caucasians (No PMID in reporting |
| [used HTR2A SSRI | (rs7997012 | of nonresponse | strategy). |
| reporting strategy | GG) | to citalopram | |
| v1] | | treatment | |
| Escitalopram (SG) | CYP2C19 | Poor | Patient has increased risk of adverse effects. Patient's genotype is associated with increased plasma concentrations of |
| [used MD | | Metabolizer | citalopram at standard doses [PMID 16291715, 17625515, 20350136, 21926427]. Decreased dosages are |
| recommendations | | | recommended [Cipralex/Lexapro Product Monograph by Lundbeck]. Consider alternative medications, such as |
| Escitalopram v2] | | | fluvoxamine or paroxetine [PMID 16384813, 17008819]. |
| Escitalopram (SG) | CYP2C19 | Intermediate | Monitor patient for adverse effects [PMID]. Patient's genotype is associated with increased plasma concentrations of |
| | | Metabolizer | escitalopram at standard doses [PMID 16418702]. Avoid concurrent use of escitalopram with CYP2C19 inhibitors |
| | | | (Cipralex label). |
| Escitalopram (SG) | CYP2C19 | Ultrarapid | Patient's genotype is associated with decreased plasma concentrations of escitalopram at standard doses, which may |
| | | Metabolizer | increase risk of therapeutic failure (PMID 17625515, 21926427). Consider alternative medications, such as fluoxetine |
| | | | or paroxetine (PMID 16384813, 17008819). |
| Fluoxetine (RC) | SLC6A4 | Decreased risk | Patient has decreased risk of adverse effects if treated with fluoxetine for major depressive disorder (PMID |
| [used “SLC6A4 | | of adverse | 18982004). |
| Reporting Strategy | | effects | |
| v4_0214a”] | | | |
| Fluoxetine (RC) | SLC6A4 | Increased risk | Patient has increased risk of adverse effects, such as headache, nausea, drowsiness, agitation, sexual dysfunction or |
| | | of adverse | weight gain, if treated with fluoxetine for major depressive disorder (PMID 18982004, |
| | | effects | http://www.nimh.nih.gov/health/publications/mental-health-medications/nimh-mental-health-medications.pdf). |
| Fluvoxamine (SG) | CYP2D6 | Poor | Patient has increased risk of adverse effects, such as gastrointestinal side effects and paroxysmal supraventricular |
| [used “MD | | Metabolizer | tachycardia (PMID 8823236, 9174682, 16205777). Patient's genotype is associated with increased plasma |
| recommendations | | | concentrations of fluvoxamine (PMID 20547595, 18978520, 8823236, 9174682, 11907488, Fluvoxamine Maleate |
| Fluvoxamine v2] | | | label). |
| Fluvoxamine (SG) | CYP2D6 | Intermediate | Patient may have increased plasma concentrations of fluvoxamine, which may increase risk of adverse effects, such as |
| | | Metabolizer | gastrointestinal side effects and paroxysmal supraventricular tachycardia (PMID 8823236, 9174682, 16205777). |
| Fluvoxamine (RC) | SLC6A4 | Decreased risk | Patient has decreased risk of adverse effects if treated with fluvoxamine for major depressive disorder (PMID |
| [used “SLC6A4 | | of adverse | 18982004). |
| Reporting Strategy | | effects | |
| v4_0214a”] | | | |
| Fluvoxamine (RC) | SLC6A4 | Increased risk | Patient has increased risk of adverse effects, such as headache, nausea, drowsiness, agitation, sexual dysfunction or |
| | | of adverse | weight gain, if treated with fluvoxamine for major depressive disorder (PMID 18982004, |
| | | effects | http://www.nimh.nih.gov/health/publications/mental-health-medications/nimh-mental-health-medications.pdf). |
| Fluvoxamine (RC) | HTR2A | Decreased risk | Patient has decreased risk of adverse effects, such as headache, nausea, drowsiness, agitation, sexual dysfunction or |
| | (rs6311 AA) | of adverse | weight gain, if treated with fluvoxamine (PMID 16205777, PMID 18982004, |
| | | effects | http://www.nimh.nih.gov/health/publications/mental-health-medications/nimh-mental-health-medications.pdf). |
| Paroxetine (SG) | CYP2D6 | Poor | Patient may have increased risk of drug-drug interactions [PMID 16476833] and sexual dysfunction [PMID 12870705]. |
| | | Metabolizer | Patient's genotype is associated with increased plasma concentrations of paroxetine at standard doses [PMID |
| | | | 1531950, 10824636, 14639062, 19743889]. In a case study of a newborn CYP2D6 poor metabolizer (CYP2D6 *4/*4), |
| | | | severe adverse effects were observed after the mother was exposed to paroxetine during late pregnancy [PMID |
| | | | 15570195]. Avoid drugs metabolized by CYP2D6 (Paxil label). |
| Paroxetine (SG) | CYP2D6 | Intermediate | Patient's genotype is associated with increased plasma concentrations of paroxetine at standard doses [PMID |
| | | Metabolizer | 16423440, 19743889, 10824636]. Patient is at risk of phenotype conversion if treated with standard doses of |
| | | | paroxetine, which may increase risk of sexual dysfunction (PMID 8880055, 12870705, 1531950). |
| Paroxetine (SG) | CYP2D6 | Ultrarapid | Patient's genotype is associated with extremely low plasma concentrations of paroxetine at standard doses (PMID |
| | | Metabolizer | 14639062, 16633156, 18641553, 19743889), which may increase risk of therapeutic failure. Consider alternative |
| | | | medications, such as citalopram or sertraline (PWG). |
| Paroxetine (RC) | SLC6A4 | Decreased risk | Patient has decreased risk of adverse effects if treated with paroxetine for major depressive disorder (PMID |
| [used “SLC6A4 | | of adverse | 18982004). |
| Reporting Strategy | | effects | |
| v4_0214a”] | | | |
| Paroxetine (RC) | SLC6A4 | Increased risk | Patient has increased risk of adverse effects, such as headache, nausea, drowsiness, agitation, sexual dysfunction or |
| | | of adverse | weight gain, if treated with paroxetine for major depressive disorder (PMID 18982004, |
| | | effects | http://www.nimh.nih.gov/health/publications/mental-health-medications/nimh-mental-health-medications.pdf). |
| Paroxetine (RC) | HTR2A | Increased risk | Patient has increased risk of adverse effects, such as headache, nausea, drowsiness, agitation, sexual dysfunction or |
| | | of adverse | weight gain, if treated with paroxetine (PMID 14514498, PMID 16874005, PMID 18982004, |
| | | effects | http://www.nimh.nih.gov/health/publications/mental-health-medications/nimh-mental-health-medications.pdf). |
| Sertraline (SG) | CYP2C19 | Poor | Patient's genotype is associated with increased plasma concentrations of sertraline at standard doses (PMID |
| | | Metabolizer | 18677622, 11452243). Consider monitoring patient for adverse effects (PMID 16384813) or consider alternative |
| | | | medications, such as fluvoxamine, paroxetine or, in CYP2D6 poor metabolizers, bupropion or mirtazapine (PMID |
| | | | 16384813). |
| Sertraline (SG) | CYP2C19 | Intermediate | Patient may have increased plasma concentrations of sertraline at standard doses (PMID 18677622, 11452243). |
| | | Metabolizer | Consider monitoring patient for adverse effects (PMID 11452243). Avoid concurrent use of sertraline with CYP2C19 |
| | | | inhibitors (www.medicines.org.uk/emc/medicine/1467, PMID 19172438). |
| TCAs | | | |
| Amitriptyline (DZ) | CYP2D6 | Poor | Patient has increased risk of adverse effects (PMID 15590749). Patient's genotype is associated with increased plasma |
| | | Metabolizer | concentrations of amitriptyline and its active metabolite, nortriptyline, at standard doses (PMID 1546384, PMID |
| | | | 3571939). Consider monitoring amitriptyline and nortriptyline levels or consider alternative medications that are not |
| | | | primarily metabolized by the CYP2D6 enzyme (PMID 21412232). Concurrent use of amitriptyline with CYP2C19 |
| | | | inducers may further increase risk of adverse effects (PMID 15590749). |
| Amitriptyline (DZ) | CYP2D6 | Intermediate | Patient has increased risk of adverse effects (PMID 15590749). Patient may have increased plasma concentrations of |
| | | Metabolizer | amitriptyline and its active metabolite, nortriptyline, at standard doses (PMID 1546384, PMID 3571939). Consider |
| | | | monitoring amitriptyline and nortriptyline levels or consider alternative medications that are not primarily |
| | | | metabolized by the CYP2D6 enzyme (PMID 21412232). Concurrent use of amitriptyline with CYP2C19 inducers or |
| | | | CYP2D6 inhibitors may increase risk of adverse effects (PMID 15590749). |
| Amitriptyline (DZ) | CYP2D6 | Ultrarapid | Patient may have decreased plasma concentrations of amitriptyline and its active metabolite, nortriptyline, at |
| | | Metabolizer | standard doses (PMID 1546384, PMID 3571939). Consider monitoring amitriptyline and nortriptyline levels, or |
| | | | consider alternative medications that are not primarily metabolized by the CYP2D6 enzyme (PMID 21412232). |
| Clomipramine (SG) | CYP2D6 | Poor | Patient has increased risk of adverse effects (PMID 2741190, 16871470). Patient's genotype is associated with |
| | | metabolizer | increased combined plasma concentrations of clomipramine and desmethylclomipramine (PMID 10460069, 2741190). |
| | | | Consider monitoring clomipramine and desmethylclomipramine plasma concentrations and consider decreased |
| | | | dosages (PMID 21412232, 16871470, 2741190, www.pharmgkb.org/drug/PA449048). |
| Clomipramine (SG) | CYP2D6 | Intermediate | Patient may have increased combined plasma concentrations of clomipramine and desmethylclomipramine. Consider |
| | | Metabolizer | monitoring clomipramine and desmethylclomipramine plasma concentrations (PWG). Patient may be at increased risk |
| | | | of conversion to a poor metabolizer, which may increase risk of adverse effects [PMID 15252821]. |
| Clomipramine (SG) | CYP2D6 | Ultrarapid | Patient has increased risk of therapeutic failure [PMID 8093319, 9562213]. Patient's genotype is associated with |
| | | Metabolizer | decreased combined plasma concentrations of clomipramine and desmethylclomipramine (PMID 8093319, 9562213). |
| | | | Consider alternative medication, such as citalopram or sertraline (PMID 21412232). |
| Desipramine (RC) | CYP2D6 | Poor | Patient's genotype is associated with increased plasma concentrations of desipramine at standard doses (PMID |
| | | Metabolizer | 9049581, PMID 3816019, PMID 10895986), which may increase risk of adverse effects. |
| Desipramine (RC) | CYP2D6 | Intermediate | Patient may have increased plasma concentrations of desipramine at standard doses (PMID 9049581, PMID 3816019, |
| | | Metabolizer | PMID 10895986), which may increase risk of adverse effects. Consider avoiding concurrent use of desipramine with |
| | | | CYP2D6 inhibitors (Nopramin label, PMID 18691982). |
| Doxepin (SG) | CYP2D6 | Poor | Patient's genotype is associated with increased plasma concentrations of doxepin, which may increase the risk of |
| | | Metabolizer | adverse effects (PMID 12360109). Consider reducing the dose (PMID 21412232). |
| Doxepin (SG) | CYP2D6 | Intermediate | Patient may have increased plasma concentrations of doxepin, which may increase the risk of adverse effects. |
| | | Metabolizer | Consider reducing the dose (PMID 21412232). |
| Doxepin (SG) | CYP2D6 | Ultrarapid | Patient's genotype is associated with decreased plasma concentrations of doxepin. Consider alternative medications, |
| | | Metabolizer | such as citalopram or sertraline, or increasing the dose (PWG). |
| Imipramine (RC) | CYP2D6 | Poor | Patient's genotype is associated with increased combined plasma concentrations of imipramine and its active |
| | | Metabolizer | metabolite, desipramine, at standard doses (PMID 17667959, PMID 9049581), which may increase risk of adverse |
| | | | effects. |
| Imipramine (RC) | CYP2D6 | Intermediate | Patient may have increased combined plasma concentrations of imipramine and its active metabolite, desipramine, at |
| | | Metabolizer | standard doses (PMID 17667959, PMID 9049581), which may increase risk of adverse effects. Consider avoiding |
| | | | concurrent use of desipramine with CYP2D6 inhibitors (Tofranil label, PMID 189691982). |
| Imipramine (RC) | CYP2D6 | Ultrarapid | Patient may have slightly lower combined plasma concentrations of imipramine and its bioactive metabolite, |
| | | Metabolizer | desipramine, at standard doses (PMID 17667959). |
| Nortriptyline (DZ) | CYP2D6 | Poor | Patient's genotype is associated with increased plasma concentrations of nortriptyline at standard doses (PMID |
| | | Metabolizer | 2815225, PMID 9585799). Consider monitoring the patient's nortriptyline plasma levels, dose adjustments or |
| | | | alternative medications that are not primarily metabolized by the CYP2D6 enzyme (PMID 15590749, PMID 21412232). |
| Nortriptyline (DZ) | CYP2D6 | Intermediate | Patient's genotype is associated with increased plasma concentrations of nortriptyline at standard doses (PMID |
| | | Metabolizer | 9797795, PMID 10770451, PMID 16778723). Consider monitoring the patient's nortriptyline plasma levels, dose |
| | | | adjustments or alternative medications that are not primarily metabolized by the CYP2D6 enzyme (PMID 15590749, |
| | | | PMID 21412232). Concurrent use of nortriptyline with CYP2D6 inhibitors may further increase plasma concentrations |
| | | | of nortriptyline. |
| Nortriptyline (DZ) | CYP2D6 | Ultrarapid | Patient's genotype is associated with decreased plasma concentrations of nortriptyline at standard doses (PMID |
| | | Metabolizer | 4082245, PMID 9585799, PMID 11673748). Consider dose adjustments or alternative medications that are not |
| | | | primarily metabolized by the CYP2D6 enzyme (PMID 21412232). |
| Trimipramine (SG) | CYP2D6 | Poor | Patient's genotype is associated with increased plasma concentrations of trimipramine and desmethyltrimipramine |
| | | Metabolizer | and no detectable concentrations of 2-hydroxy trimipramine (PMID 14520122, 14646691, 10774635). Therefore, the |
| | | | patient may be at increased risk of adverse effects, such as sedation (PMID 14646691). |
| Trimipramine (SG) | CYP2D6 | Intermediate | Patient's genotype is associated with increased plasma concentrations of trimipramine and desmethyltrimipramine |
| | | Metabolizer | (PMID 14520122), which may increase risk of adverse effects, such as sedation (PMID 14646691). |
| Trimipramine (SG) | CYP2D6 | Ultrarapid | Patient has increased risk of therapeutic failure (PMID 14646691). Patient's genotype is associated with decreased |
| | | Metabolizer | combined plasma concentrations of trimipramine and desmethyltrimipramine (PMID 14646691). |
| Other Antidepressants | | | |
| Buspirone | No | | |
| | associations | | |
| Duloxetine (AT) | CYP2D6 | Poor | Patient may have increased plasma concentrations of duloxetine at standard doses (PMID 17380590, 17713974). |
| | | Metabolizer | |
| Duloxetine (AT) | CYP2D6 | Intermediate | Patient may have increased plasma concentrations of duloxetine at standard doses (PMID 17380590, 17713974). |
| | | Metabolizer | |
| Mirtazapine (AT) | CYP2D6 | Poor | Patient may have increased plasma concentrations of mirtazapine at standard doses, though it is unclear if increased |
| | | Metabolizer | plasma concentrations of mirtazapine influence therapeutic benefit or the risk of adverse effects. |
| Mirtazapine (AT) | CYP2D6 | Intermediate | Patient may have increased plasma concentrations of mirtazapine, though it is unclear if increased plasma |
| | | Metabolizer | concentrations of mirtazapine influence therapeutic benefit or the risk of adverse effects. |
| Mirtazapine (AT) | CYP2D6 | Ultrarapid | Patient may have decreased plasma concentrations of mirtazapine, though it is unclear if decreased plasma |
| | | Metabolizer | concentrations of mirtazapine influence therapeutic benefit or the risk of adverse effects. |
| trazodone | No | | |
| | associations | | |
| Venlafaxine (AC) | CYP2D6 | Poor | Patient's genotype is associated with increased plasma concentrations of venlafaxine and decreased levels of the |
| | | Metabolizer | active metabolite, O-desmethylvenlafaxine, at standard doses (PMID 10192828, 10780263, 12544511, 16958828, |
| | | | 18214456, 19593180, 21288052); therefore, the patient may have an increased risk of adverse effects at standard |
| | | | doses of venlafaxine (PMID 10780263, 16958828). Preliminary evidence also points to a reduced therapeutic effect at |
| | | | standard doses of venlafaxine in patients of this genotype (PMID 20441720). Consider alternative medications, such as |
| | | | citalopram or sertraline (PMID 21412232). |
| Venlafaxine (AC) | CYP2D6 | Intermediate | Patient's genotype is associated with increased plasma concentrations of venlafaxine at standard doses (PMID |
| | | Metabolizer | 10233212, 10877013), which may increase risk of adverse effects (PMID 17803873). Consider alternative medications, |
| | | | such as citalopram or sertraline (PMID 21412232). |
| Venlafaxine (AC) | CYP2D6 | Ultrarapid | Patient's genotype is associated with increased plasma levels of the active metabolite, O-desmethylvenlafaxine, and |
| | | Metabolizer | decreased levels of venlafaxine at standard doses (PMID 16958828). |
| Venlafaxine (RC) | SLC6A4 | Increased | Patient's genotype is associated with an increased response to venlafaxine (PMID 20664233, PMID 22907732). |
| [used “SLC6A4 | | response to | |
| Reporting Strategy | | venlafaxine | |
| v4_0214a”] | | | |
| Venlafaxine (RC) | SLC6A4 | Decreased | Patient's genotype is associated with a decreased response to venlafaxine (PMID 20664233, PMID 22907732). |
| | | response to | |
| | | venlafaxine | |
| Atypical Antipsychotics | | | |
| Aripiprazole (AT) | CYP2D6 | Poor | Patient's genotype is associated with increased plasma concentrations of aripiprazole at standard doses (PMID |
| | | Metabolizer | 21157400, 17828532), which may increase the risk of adverse effects (PMID 17202571). Consider reducing the |
| | | | maximum dose to 10 mg/day (PMID 21412232). |
| Aripiprazole (AT) | CYP2D6 | Intermediate | Patient's genotype is associated with increased plasma concentrations of aripiprazole at standard doses (PMID |
| | | Metabolizer | 21157400, 17828532). |
| Aripiprazole (AT) | CYP2D6 | Ultrarapid | Patient's genotype is associated with decreased plasma concentrations of aripiprazole at standard doses (PMID |
| | | Metabolizer | 21157400, 17828532). |
| Aripiprazole (AT) | HTR2C | Reduced Risk of | Patient has decreased risk of weight gain if treated with atypical antipsychotics, including aripiprazole (PMID |
| | | Weight Gain | 19636338, 15666332, 19434072, 21510767, 15864111, and 21121776). |
| Asenapine (AT) | HTR2C | Reduced weight | Patient has decreased risk of weight gain if treated with atypical antipsychotics, including asenapine (PMID 19636338, |
| | | gain | 15666332, 19434072, 21510767, 15864111, and 21121776). |
| Clozapine (AT) | CYP1A2 | Fast | Patient's genotype is associated with decreased plasma concentrations of clozapine in smokers, which may lead to |
| | | metabolizer | decreased efficacy at standard doses (PMID 11763009, 15206669). This recommendation does not apply to patients of |
| | | | Asian ancestry (PMID 17370067). |
| Clozapine (AT) | HTR2C | Reduced weight | Patient has decreased risk of weight gain if treated with atypical antipsychotics, including clozapine (PMID 19636338, |
| | | gain | 15666332, 19434072, 21510767, 15864111, and 21121776). |
| Iloperidone (SG) | CYP2D6 | Poor | Patient has increased risk of adverse effects, such as prolonged QT interval (Iloperidone label). Consider a 50% |
| | | metabolizer | reduction in dosage (Iloperidone label). Patient's genotype is associated with increased plasma concentrations of |
| | | | iloperidone and its active metabolite (Posters 1 and 2). |
| Iloperidone (SG) | CYP2D6 | Intermediate | Patient may have increased plasma concentrations of iloperidone and its active metabolite. |
| | | metabolizer | |
| Iloperidone (AT) | HTR2C | Reduced weight | Patient has decreased risk of weight gain if treated with atypical antipsychotics, including iloperidone (PMID |
| | | gain | 19636338, 15666332, 19434072, 21510767, 15864111, and 21121776). |
| Lurasidone (AT) | HTR2C | Reduced weight | Patient has decreased risk of weight gain if treated with atypical antipsychotics, including lurasidone (PMID 19636338, |
| | | gain | 15666332, 19434072, 21510767, 15864111, and 21121776). |
| Olanzapine (AT) | CYP1A2 | Fast | Patient's genotype is associated with decreased plasma concentrations of olanzapine, which may lead to decreased |
| | | metabolizer | efficacy (PMID 19636338). This recommendation does not apply to patients of Asian ancestry (PMID 17370067). |
| Olanzapine (AT) | HTR2C | Reduced weight | Patient has decreased risk of weight gain if treated with atypical antipsychotics, including olanzapine (PMID 19636338, |
| | | gain | 15666332, 19434072, 21510767, 15864111, and 21121776). |
| Paliperidone (AT) | HTR2C | Reduced weight | Patient has decreased risk of weight gain if treated with atypical antipsychotics, including paliperidone (PMID |
| | | gain | 19636338, 15666332, 19434072, 21510767, 15864111, and 21121776). |
| Quetiapine (AT) | HTR2C | Reduced weight | Patient has decreased risk of weight gain if treated with atypical antipsychotics, including quetiapine (PMID 19636338, |
| | | gain | 15666332, 19434072, 21510767, 15864111, and 21121776). |
| Risperidone (AT) | CYP2D6 | Poor | Patient's genotype is associated with an increased plasma risperidone:9-OH-risperidone ratio, which may increase the |
| | | Metabolizer | risk of adverse effects (PMID 15669884). Consider an alternative drug, such as quetiapine, olanzapine or clozapine, or |
| | | | monitor patient for adverse events and adjust dosage accordingly (PMID 21412232). |
| Risperidone (AT) | CYP2D6 | Intermediate | Patient's genotype is associated with an increased plasma risperidone:9-OH-risperidone ratio, which may increase the |
| | | metabolizer | risk of adverse effects (PMID 15669884). Consider an alternative drug, such as quetiapine, olanzapine or clozapine, or |
| | | | monitor patient for adverse events and adjust dosage accordingly (PMID 21412232). |
| Risperidone (AT) | CYP2D6 | Ultrarapid | Patient's genotype is associated with a decreased plasma risperidone:9-OH-risperidone ratio. Consider an alternative |
| | | metabolizer | drug, such as quetiapine, olanzapine or clozapine, or monitor patient for decreased response and adjust dosage |
| | | | accordingly (PMID 21412232). |
| Risperidone (AT) | HTR2C | Reduced weight | Patient has decreased risk of weight gain if treated with atypical antipsychotics, including risperidone (PMID |
| | | gain | 19636338, 15666332, 19434072, 21510767, 15864111, and 21121776). |
| Risperidone (AT) | DRD2 | Reduced | Patient has a reduced likelihood of responding to antipsychotic treatment (PMID 20194480). Other genetic factors |
| | | Benefit | may also affect clinical response to antipsychotics. |
| Ziprasidone (AT) | HTR2C | Reduced weight | Patient has decreased risk of weight gain if treated with atypical antipsychotics, including ziprasidone (PMID |
| | | gain | 19636338, 15666332, 19434072, 21510767, 15864111, and 21121776). |
| Typical Antipsychotics | | | |
| Haloperidol (SG) | CYP2D6 | Poor | Patient has increased risk of extrapyramidal symptoms and other adverse effects. Consider reducing the dose or |
| | | metabolizer | alternative medications that are not primarily metabolized by the CYP2D6 enzyme, such as pimozide, flupenthixol, |
| | | | fluphenazine, quetiapine, olanzapine or clozapine (PMID 21412232). Patient's genotype is associated with increased |
| | | | plasma concentrations of reduced haloperidol (PMID 12386646, 10519444, 1585408, 12746736). |
| Haloperidol (SG) | CYP2D6 | Intermediate | Patient may have increased plasma concentrations of haloperidol (PMID 12386646, 10519444, 10096261). |
| | | metabolizer | |
| Haloperidol (SG) | CYP2D6 | Ultrarapid | In one study, individuals with this patient's CYP2D6 metabolizer status had a slightly increased risk of extrapyramidal |
| | | metabolizer | symptoms if treated with haloperidol (PMID 12386646). Consider an alternative drug (e.g., pimozide, fluphenthixol, |
| | | | fluphenazine, quetiapine, olanzapine or clozapine) or monitor patient for haloperidol concentrations and adjust |
| | | | dosage accordingly (PMID 21412232). |
| Perphenazine (AT) | CYP2D6 | Poor | Patient's genotype is associated with increased plasma concentrations of perphenazine (PMID 17429316, 8612387, |
| | | metabolizer | 8689810), which may increase risk of adverse effects (FDA approved perphenazine drug label, PMID 9333110, |
| | | | 7491387). |
| Perphenazine (AT) | CYP2D6 | Intermediate | Patient's genotype is associated with increased plasma concentrations of perphenazine (PMID 17429316, 8612387), |
| | | metabolizer | which may increase risk of adverse effects. |
| Perphenazine (AT) | CYP2D6 | Ultrarapid | Patient's genotype is associated with decreased plasma concentrations of perphenazine. |
| | | metabolizer | |
| Thioridazine (SG) | CYP2D6 | Poor | Patient has increased risk of cardiac side effects, such as prolonged QT interval and arrhythmia (PMID 12503836, |
| | | metabolizer | Thioridazine label). Avoid the use of thioridazine in this patient (drug label). Patient's genotype is associated with |
| | | | increased plasma concentrations of thioridazine at standard doses (PMID 17460606, 12682803). |
| Thioridazine (SG) | CYP2D6 | Intermediate | Patient may have decreased CYP2D6 enzyme activity at standard doses. The CYP2D6 enzyme contributes to the |
| | | metabolizer | metabolism of cardiotoxic thioridazine to inactive metabolites [from curator report]. Caution should be |
| | | | exercised when using thioridazine in this patient. |
| Zuclopenthixol (AT) | CYP2D6 | Poor | Patient's genotype is associated with increased plasma concentrations of zuclopenthixol (PMID 1927573, 8946657, |
| | | metabolizer | 12107620, 8612387, 20946203), which may increase risk of adverse effects (PMID 20175668, 12107620). Consider |
| | | | reducing the dose by 50% or consider alternative medications, such as pimozide, flupenthixol, fluphenazine, |
| | | | quetiapine, olanzapine or clozapine (PMID 21412232). |
| Zuclopenthixol (AT) | CYP2D6 | Intermediate | Patient may have increased plasma concentrations of zuclopenthixol (PMID 1927573, 8946657, 12107620, 8612387, |
| | | metabolizer | 20946203), which may increase risk of adverse effects (PMID 20175668, 12107620). Consider reducing the dose by |
| | | | 25% or consider alternative medications, such as pimozide, flupenthixol, fluphenazine, quetiapine, olanzapine or |
| | | | clozapine (PMID 21412232). |
| Zuclopenthixol (AT) | CYP2D6 | Ultrarapid | Consider monitoring patient for low zuclopenthixol plasma concentration or consider an alternative drug, such as |
| | | metabolizer | fluphenthixol, quetiapine, olanzapine or clozapine (PMID 21412232). |
| Mood Stabilizers | | | |
| Carbamazepine | HLA- | Hypersensitive | Patient has increased risk of developing Stevens-Johnson syndrome or toxic epidermal necrolysis during |
| (AC) | B*1502 | | treatment with carbamazepine. Patient is likely to have at least one copy of the HLA-B*1502 allele that is |
| | | | associated with increased risk (PMID 20235791, 18785891). The use of carbamazepine in this patient |
| | | | should be carefully considered. Patients who test positive for the HLA-B*1502 allele and have been taking |
| | | | carbamazepine for more than a few months without developing skin reactions have a low risk of becoming |
| | | | hypersensitive. HLA-B*1502-positive patients could also be advised to avoid related anticonvulsants, such |
| | | | as phenytoin and oxcarbazepine. |
| | | | This genetic test is most applicable to patients of Han Chinese descent. If clinically indicated, patients of |
| | | | other Asian ethnicities could be advised to undergo HLA sequencing to assess their risk of carbamazepine |
| | | | hypersensitivity. Other HLA alleles have been shown to be associated with carbamazepine hypersensitivity |
| | | | in people of Caucasian and Japanese descent, in whom HLA-B*1502 is largely absent. |
| Carbamazepine | HLA- | Unknown | Patient's risk of developing carbamazepine hypersensitivity cannot be determined from the genotype |
| (AC) | B*1502 | | results. It cannot be determined whether or not this patient has the HLA-B*1502 allele that is associated |
| | | | with serious skin reactions, such as Stevens-Johnson syndrome or toxic epidermal necrolysis, if treated with |
| | | | carbamazepine. This genetic test is most applicable to patients of Han Chinese descent. If clinically |
| | | | indicated, patients of Asian descent, including Han Chinese who receive an “Unknown” outcome, could be |
| | | | advised to undergo HLA sequencing to assess their risk of carbamazepine hypersensitivity. Other HLA alleles |
| | | | have been shown to be associated with carbamazepine hypersensitivity in people of Caucasian and |
| | | | Japanese descent, in whom HLA-B*1502 is largely absent. |
| Gabapentin | No | | |
| | associations | | |
| Lamotrigine (AT) | UGT1A4 | Fast | Patient may have decreased plasma concentrations of lamotrigine at standard doses (PMID 21601426). Co- |
| | | metabolizer | administered drugs may also affect lamotrigine plasma levels by inhibiting or inducing enzymes involved in |
| | | | lamotrigine metabolism |
| Lamotrigine (AT) | HLAB*1502 | Hypersensitive | Patient may have increased risk of developing Stevens-Johnson syndrome or toxic epidermal necrolysis |
| | | | during treatment with lamotrigine. Patient is likely to have at least one copy of the HLA-B*1502 allele that |
| | | | is associated with increased risk (PMID 20235791, 21071176). The use of lamotrigine in this patient should |
| | | | be carefully considered. Patients who test positive for the HLA-B*1502 allele and have been taking |
| | | | lamotrigine for more than a few months without developing skin reactions have a low risk of becoming |
| | | | hypersensitive. HLA-B*1502-positive patients could also be advised to avoid related anticonvulsants, such |
| | | | as phenytoin and oxcarbazepine. |
| | | | This genetic test is most applicable to patients of Han Chinese descent. If clinically indicated, patients of |
| | | | other Asian ethnicities could be advised to undergo HLA sequencing to assess their risk of lamotrigine |
| | | | hypersensitivity. Other HLA alleles have been shown to be associated with lamotrigine hypersensitivity in |
| | | | people of Caucasian and Japanese descent, in whom HLA-B*1502 is largely absent. |
| Lamotrigine (AT) | HLAB*1502 | Unknown | Patient has increased risk of developing Stevens-Johnson syndrome or toxic epidermal necrolysis during |
| | | | treatment with oxcarbazepine. Patient is likely to have at least one copy of the HLA-B*1502 allele that is |
| | | | associated with increased risk (PMID 20235791, 18785891). This genetic test is most applicable to patients |
| | | | of Han Chinese descent. If clinically indicated, patients of other Asian ethnicities could be advised to |
| | | | undergo HLA sequencing to assess their risk of oxcarbazepine hypersensitivity. Other HLA alleles have been |
| | | | shown to be associated with oxcarbazepine hypersensitivity in people of Caucasian and Japanese descent, |
| | | | in whom HLA-B*1502 is largely absent. |
| Oxcarbazepine | HLAB*1502 | Hypersensitive | Patient has increased risk of developing Stevens-Johnson syndrome or toxic epidermal necrolysis during |
| (AT) | | | treatment with oxcarbazepine. Patient is likely to have at least one copy of the HLA-B*1502 allele that is |
| | | | associated with increased risk (PMID 20235791, 18785891). The use of oxcarbazepine in this patient should |
| | | | be carefully considered. Patients who test positive for the HLA-B*1502 allele and have been taking |
| | | | oxcarbazepine for more than a few months without developing skin reactions have a low risk of becoming |
| | | | hypersensitive. HLA-B*1502-positive patients could also be advised to avoid related anticonvulsants, such |
| | | | as phenytoin and oxcarbazepine. |
| | | | This genetic test is most applicable to patients of Han Chinese descent. If clinically indicated, patients of |
| | | | other Asian ethnicities could be advised to undergo HLA sequencing to assess their risk of oxcarbazepine |
| | | | hypersensitivity. Other HLA alleles have been shown to be associated with oxcarbazepine hypersensitivity |
| | | | in people of Caucasian and Japanese descent, in whom HLA-B*1502 is largely absent. |
| Oxcarbazepine | HLAB*1502 | Unknown | Patient's risk of developing oxcarbazepine hypersensitivity cannot be determined from the genotype |
| (AT) | | | results. It cannot be determined whether or not this patient has the HLA-B*1502 allele that is associated |
| | | | with serious skin reactions, such as Stevens-Johnson syndrome or toxic epidermal necrolysis, if treated with |
| | | | oxcarbazepine. This genetic test is most applicable to patients of Han Chinese descent. If clinically indicated, |
| | | | patients of Asian descent, including Han Chinese who receive an “Unknown” outcome, could be advised to |
| | | | undergo HLA sequencing to assess their risk of oxcarbazepine hypersensitivity. Other HLA alleles have been |
| | | | shown to be associated with oxcarbazepine hypersensitivity in people of Caucasian and Japanese descent, |
| | | | in whom HLA-B*1502 is largely absent. |
| Topiramate | No | | |
| | associations | | |
| Valproic Acid | No | | |
| | associations | | |

1-30. (canceled)

31. A method for determining the likely response to a psychiatric medication in a human individual in need thereof, comprising:
a) providing a nucleic acid sample from a human individual suspected of suffering from a mental disorder;
b) contacting the nucleic acid sample with a prescribed panel of synthetic probes, at least three of which are complementary to a nucleotide sequence of one or more target genetic polymorphisms set forth in TABLEs 1-4, wherein at least one of the synthetic probes is specific to a genetic polymorphism within a gene encoding UDG-glucuronosyltransferase (UGT), and wherein the presence of any one of the one or more target genetic polymorphisms in the nucleic acid sample creates synthetic probe-nucleic acid complexes specific for the target genetic polymorphisms present in the nucleic acid sample;
c) detecting the synthetic probe-nucleic acid complexes created in step (b), thereby identifying one or more genetic polymorphisms present in the individual,
d) assigning three categorical grades to the individual, based on the individual's genetic profile comprising the one or more genetic polymorphisms identified in step (c):
(i) a first categorical grade to the individual's likely ability to metabolize the psychiatric medication, wherein the individual is assigned to a default categorical grade of “Use As Directed By Manufacturer” if no genetic polymorphism is identified in the individual, or assigned to a more precautionary grade selected from the group consisting of the following grades, ranked from least-to-most precautionary level: “Preferential Use”, “Use with Limitations”, and “May Cause Serious Adverse Events,” if at least one of the polymorphisms identified in the individual is indicated in TABLEs 1-4 as associated with drug metabolism,
(ii) a second categorical grade for a potential efficacy of the psychiatric medication with respect to the individual, wherein the individual is assigned to a default categorical grade of “Use As Directed By Manufacturer” if no genetic polymorphism is identified in the individual, or assigned to a more precautionary grade selected from the group consisting of the following grades, ranked from least-to-most precautionary level: “Preferential Use”, “Use with Limitations”, and “May Cause Serious Adverse Events,” if at least one of the polymorphisms identified in the individual is indicated in TABLEs 1-4 as associated with drug efficacy, and
(iii) a third categorical grade to the propensity for the individual to have a negative adverse reaction to the psychiatric medication, wherein the individual is assigned to a default categorical grade of “Use As Directed By Manufacturer” if no genetic polymorphism is identified in the individual, or assigned to a more precautionary grade selected from the group consisting of the following grades, ranked from least-to-most precautionary level: “Preferential Use”, “Use with Limitations”, and “May Cause Serious Adverse Events,” if at least one of the polymorphisms identified in the individual is indicated in TABLEs 1-4 as associated with side effects; and
e) comparing the first, second, and third categorical grades assigned to the individual in step (d) to determine the categorical grade corresponding to the most precautionary level among the first, second, and third categorical grades as the likely response to the psychiatric medication for the individual.

32. The method of claim 31, further comprising identifying genetic polymorphisms in the individual to assign a fourth categorical grade to the individual's susceptibility to the mental disorder.

33. The method of claim 32, wherein the mental disorder is selected from the group consisting of mood disorders, psychotic disorders, personality disorders, anxiety disorders, substance-related disorders, childhood disorders, dementia, autistic disorders, adjustment disorders, delirium, multi-infarct dementia, eating disorders, addictive behaviors, ADHD, PTSD, and Tourette's disorder.

34. The method of claim 31, further comprising providing a recommendation of the psychiatric medication's use for the individual based on the determined likely response to the psychiatric medication, wherein the recommendation is selected from the group consisting of:
Use as Directed By Manufacturer,
Preferential Use, and
Precautionary Use.

35. The method of claim 31, wherein the psychiatric medication is selected from the group consisting of antidepressants, antipsychotics, stimulants, anxiolytics, mood stabilizers, and depressants.

36. The method of claim 35, wherein the psychiatric medication is selected from the group consisting of lamotrigine, Quetiapine, carbamazepine, aripiprazole, olanzapine, risperidone, ziprasidone, citalopram, fluoxetine, fluvoxamine, paroxetine, sertraline, mirtazapine, oxcarbazepine, clozapine, duloxetine, venlafaxine, amitriptyline, nortriptyline, imipramine, escitalopram, clomipramine, desipramine, doxepin, trimipramine, iloperidone, asenapine, lurasidone, paliperidone, haloperidol, perphenazine, thioridazine, lithium, zuclopenthixol, valproic acid, buspirone, gabapentin, topiramate, trazodone, chlorpromazine, fluphenazine, loxapine, thiothixene, trifluoperazine, bupropion, amphetamine, modafinil, phenytoin, droperidol, diazepam, nordazepam, temazepam, triazolam, flurazepam, bromazepam, clobazam, etizolam, alprazolam, lorazepam, midazolam, oxazepam, clonazepam, and protriptyline.

37. The method of claim 31, wherein the individual's genetic profile comprises genetic variations in the following panels of genes:
a panel of at least one gene that affects the rate of drug metabolism,
a panel of genes that affect a potential efficacy of the psychiatric medication with respect to the individual, and
a panel of genes that affect the propensity for the individual to have a negative adverse reaction to the psychiatric medication.

38. The method of claim 37, wherein the panel for genes affecting drug metabolism comprises at least one gene that affects biochemical modification of pharmaceutical substances or xenobiotics, the panel for genes affecting efficacy comprises at least one neurotransmitter modulating gene, and the panel for genes affecting adverse reaction comprises at least one gene associated with an undesired reaction selected from the group consisting of 1) a mechanism based reaction and 2) an idiosyncratic, “unpredictable” reaction which is unrelated to the primary pharmacologic action of the psychiatric medication.

39. The method of claim 37, wherein the panel of genes for affecting drug metabolism comprises at least one cytochrome P450 gene,

40. The method of claim 39, wherein the panel for genes for affecting drug metabolism further comprises at least one gene selected from UDP-glucuronosyltransferase, 5,10-methylenetetrahydrofolate reductase, and ATP-binding cassette (ABC) transporters.

41. The method of claim 37, wherein the panel of genes for affecting drug metabolism comprises at least one gene selected from the group consisting of CYP1A1, CYP2A6, CYP2C9, CYP2D6, CYP2E1, CYP3A5, CYP1A2, CYP1B1, CYP2B6, CYP2C8, CYP2C18, CYP2C19, CYP2E1, CYP3A4, CYP3A5, UGT1A4, UGT1A1, UGT1A9, UGT2B4, UGT2B7, UGT2B15, NAT1, NAT2, EPHX1, MTHFR, and ABCB1.

42. The method of claim 37, wherein the panel of genes for affecting a medication's potential efficacy comprises at least one gene for a serotonin transporter or serotonin receptor gene.

43. The method of claim 37, wherein the panel of genes for affecting a medication's potential efficacy comprises one or more dopamine transporter gene or dopamine receptor genes.

44. The method of claim 37, wherein the panel of genes for affecting drug metabolism comprises CYP2D6, CYP2B6, CYP2C19, and UGT1A4 genes;
wherein the panel of genes for affecting efficacy comprises the serotonin transporter gene (SLC6A4), the serotonin receptor 2A gene (HTR2A) and dopamine receptor D2 (DRD2); and
wherein the panel of genes for affecting adverse reactions comprises the serotonin receptor 2A (HTR2A), the serotonin gene 2C (HTR2C) and the major histocompatibility complex, class I, B (HLA-B).

45. A kit for determining the likely response to a psychiatric medication in a human individual, wherein the kit comprises a gene panel comprising one or more probes or primers for genotyping one or more of gene groups:
(a) CYP2D6, CYP3A4, SLC6A4, CYP2C19, and HTR2A genes;
(b) CYP2D6, CYP3A4, CYP2B6, and SLC6A4 genes;
(c) CYP2D6 and CYP2C19 genes;
(d) CYP2D6, CYP3A4, CYP2B6, and SLC6A4 genes;
(e) CYP2D6 gene;
(f) CYP2C19 and CYP3A5 genes;
(g) HLA-A, HLA-B, UGT1A4, POLG, and CYP2C9 genes; and
(h) CYP2D6, CYP1A2, HTR2C, and DRD2 genes;

46. The kit of claim 45, wherein the psychiatric medication is an antidepressant, an ADHD medication, a Benzodiazepine, a mood stabilizer, or an antipsychotic.

47. The kit of claim 45, wherein the gene panel is for analyzing a mood stabilizer and comprises HLA-A, HLA-B, UGT1A4, POLG, and CYP2C9 genes.

48. The kit of claim 45, wherein the gene panel is for analyzing an ADHD medication or an atypical antipsychotics, and comprises CYP2D6 gene.

49. The kit of claim 45, wherein the kit further comprises a gene panel for analyzing a Benzodiazepine and comprises CYP2C19 and CYP3A5 genes.

50. The kit of claim 45, wherein the kit further comprises a gene panel for analyzing a typical antipsychotics and comprising CYP2D6, CYP1A2, HTR2C and DRD2 genes.
